(19) United States
(12) Patent Application Publication
(10) Pub. No.: US 2026/0325439 A1
(43) Pub. Date: Oct. 22, 2026

(54) FORMULATIONS TO ENHANCE THE SKIN BARRIER AND STIMULATE VITAMIN D

(71) Applicant: Colorescience, Inc., Carlsbad, CA (US)

(72) Inventors: Heather Goodchild, Panama City Beach, FL (US); Patricia McGill Boland, Houston, TX (US); Deborah Eileen Gregg Bouche, Houston, TX (US); Mellisa Ann Murphy, Katy, TX (US)

(21) Appl. No.: 19/648,735

(22) Filed: Apr. 15, 2026

Related U.S. Application Data
(60) Provisional application No. 63/789,888, filed on Apr. 16, 2025.

(51) Int. Cl. A61K 8/99; A61K 8/37; A61K 8/64; A61K 8/73; A61K 8/9789; A61K 31/417; A61K 31/702; A61K 35/00; A61K 35/747; A61K 36/33; A61K 47/02; A61K 47/06; A61K 47/10; A61K 47/14; A61K 47/26; A61K 47/36; A61K 47/42; A61K 47/46; A61Q 19/00

(52) U.S. Cl. CPC A61K 8/99; A61K 8/375; A61K 8/645; A61K 8/73; A61K 8/9789; A61K 31/417; A61K 31/702; A61K 35/747; A61K 36/33; A61K 47/02; A61K 47/06; A61K 47/10; A61K 47/14; A61K 47/26; A61K 47/36; A61K 47/42; A61K 47/46; A61Q 19/007; A61K 2035/115

(57) ABSTRACT

Topical formulations for restoring healthy skin are provided, by for example, restoring the skin barrier by maintaining a beneficially balanced microbiome and/or stimulating vitamin D based compounds without the need for damaging UV rays. Skin care sprays, cleansing and/or moisturizing formulations are provided in some embodiments in which select ingredients work together to hydrate the skin, bring balance to or otherwise improve the skin microbiome, stimulate vitamin D based compounds, and improve the barrier function of the skin.

### CROSS REFERENCE TO RELATED APPLICATIONS

This application claims priority to U.S. Provisional Application No. 63/789,888, filed Apr. 16, 2025, the entire contents of which is incorporated by reference herein.

### FIELD

The field relates generally to skin care, including those for hydrating or cleansing the skin. According to several embodiments, topical formulations that show visible beneficial dermatological (e.g., cosmetic, aesthetic) results are provided, including formulations to restore and enhance the skin barrier through a healthy microbiome and formulations that stimulate vitamin D based compounds.

### BACKGROUND

Skin care formulations, such as those for face and body, include for example, serums, lotions, and creams, are used for various purposes. Formulations can act as a cleanser, moisturizer, provide coverage, or contain vitamins. For protection, some formulations contain a sun protection factor (SPF).

### SUMMARY

Despite the large number of cosmetic formulations on the market, there remains a need for enhanced topical formulations that simultaneously replenish, rebalance, soothe and defend the skin against environmental aggressors. In one embodiment, the formulation hydrates the skin and promotes a balanced microbiome. In some embodiments, the formulation cleanses, replenishes the skin and promotes a balanced microbiome. In some embodiments, the formulation improves barrier function of the skin. In one embodiment, the formulation provides significant UV protection (such as physical UVA and UVB sun protection). In one embodiment, neither titanium dioxide nor zinc oxide is included, but the formulation nonetheless provides protection from Infrared radiation (IR), blue and/or UV light. Several embodiments stimulate vitamin D production and/or activity. Unexpectedly, according to several embodiments, the formulations provided herein produce Vitamin D based compounds without relying on damaging ultraviolet light, while supporting the microbiome and restoring the skin's barrier, with the ingredients working synergistically to achieve these benefits.

In several embodiments, formulations are provided that strengthen skin's immunity to help resist visible signs of aging and improve the appearance of smoother, firmer, and balanced skin, and include ingredients that help promote skin's natural ability to synthesize essential Vitamin D based compounds for immunity support and immediate radiance with pre-, pro-, and postbiotic benefits to support the skin's microbiome balance and barrier strength, and hydration. At least one vitamin D compound is stimulated without the adverse effects of the sun in several embodiments. The formulations provided herein have several or all of the following advantages in several embodiments: (i) soothes dull, stressed, reactive skin, (ii) hydration and luminosity, (iii) combines Vitamin D support with microbiome support for a strengthened skin barrier and improved radiance against environmental aggressors, (iv) advanced antioxidants neutralize free radicals to prevent from visible skin damage, (v) multi-layer hydration with tri-molecular hyaluronic acid, (vi) continues to improve resilience and adaptive response over time, and (vii) oil-free, non-comedogenic, non-greasy, fragrance-free, hypoallergenic, and vegan.

In several embodiments, formulations for stimulating a vitamin D based compound and enhancing the microbiome and skin barrier are provided. In some embodiments, the formulation comprises a probiotic, a postbiotic, an ingredient to stimulate a vitamin D compound, and a synthetic preservative, wherein the formulation is provided as a topical formulation and is configured for use on a user's skin for both stimulating a vitamin D based compound and enhancing the microbiome. In several embodiments, a formulation for stimulating a vitamin D based compound and supporting the microbiome of the skin comprises: amino acids, peptides and/or proteins, lithops, hyaluronic acid, prickly pear cactus extract, goji berry extract, Lactobacillus ferment, squalane and hydrolyzed pea protein. In some embodiments, the lithops is lithops plant cell nectar and the hyaluronic acid is in trimolecular form. Although vitamin D is activated, vitamin D is not included in the formulation in several embodiments. In several embodiments, the lithops extract comprises fructooligosaccharides, bioflavonoids, and glycoproteins.

In several embodiments, the formulations provided herein comprise one, two or more of a prebiotic, a probiotic and a postbiotic.

As used herein, a “prebiotic” shall be given its ordinary meaning and shall include nutrients, substrates, and other ingredients (such as nondigestible food ingredients) that feed or otherwise promote the growth of beneficial microbes.

As used herein, a “probiotic” shall be given its ordinary meaning and shall include living microbes and/or their byproducts (such as certain ferments) that have pro-biotic like activity in that, for example, they repopulate or otherwise increase the number of healthy microbes and/or that have beneficial bioactivity. As an example, a probiotic includes byproducts of bacteria or yeast (without any or any significant live microorganisms), wherein the byproducts increase beneficial microbe numbers and/or decrease harmful microbes. A probiotic also includes embodiments that contain living beneficial microbes.

As used herein, a “postbiotic” shall be given its ordinary meaning and shall include nonliving or inactive microbial cells, cell structures or byproducts of microbes that provide an environment that allows beneficial biodiversity to thrive. Postbiotics can include ferments that support a beneficial microbial environment but do not necessarily repopulate microbes.

In several embodiments, formulations are provided that comprise an Opuntia ficus-indica extract (e.g., stem extract), a Lycium barbarum extract (e.g., fruit extract), a Lactobacillus ferment, a chronopeptide or chrono-compound (including but not limited to glutamylamidoethyl imidazole), a preservative (e.g., a synthetic or modified preservative), and optionally sodium hydroxide. In some embodiments, a chronopeptide or chrono-compound such as glutamylamidoethyl imidazole activates circadian genes which transform, process, or otherwise makes bioavailable a vitamin D compound, and is provided synergistically with fructooligosaccharides, glycoproteins, and bioflavonoids that are extracted from succulents, including but not limited to lithops, to stimulate the production of a vitamin D based compound without the need for damaging UV light.

In several embodiments, a formulation is provided to increase the bioavailability of a vitamin D based compound that comprises: (i) a chrono-compound that transforms, processes, or makes bioavailable a vitamin D compound (including but not limited to imidazoles, glutamic acid compounds, aminoethylimidazole, glutamylamidoethyl imidazole, and anti-glycation compounds, (ii) a fructooligosaccharide, (iii) a glycoprotein, and (iv) a bioflavonoid. Extracts from succulents may be used, including but not limited to lithops, to stimulate the production of a vitamin D based compound without the need for damaging UV light.

Formulations for stimulating a vitamin D based compound (e.g., at least one of vitamin D and a precursor of vitamin D) are provided in several embodiments, comprising: a plurality of skin conditioning agents comprising fructooligosaccharides, glycoproteins, bioflavonoids, glycerin, and a Opuntia ficus-Indica extract (e.g. stem extract); at least one probiotic (e.g., Lactobacillus ferment) at least one postbiotic (e.g., squalane); and one or more ingredients selected from the group consisting or consisting essentially of a hydrolyzed pea protein, glutamylamidoethyl imidazole, polyglyceryl-10 oleate, sodium hydroxide, and pentylene glycol.

Formulations for stimulating a vitamin D based compound (e.g., at least one of vitamin D and a precursor of vitamin D) are provided in several embodiments, comprising at least one probiotic, at least one postbiotic, at least one ingredient to hydrate skin; and at least one ingredient to stimulate at least one of vitamin D and a precursor of vitamin D.

In some embodiments, the formulations comprises one or more of the following: Opuntia ficus-Indica extract in an amount between 0.001-2% of the formulation, Lycium barbarum fruit extract in an amount between 0.001-2% of the formulation, glutamylamidoethyl imidazole in an amount between 0.001-2% of the formulation, Lactobacillus ferment in an amount between 0.001% and 3% of the formulation and squalane in an amount between 0.5% and 3% of the formulation. One or more of glucose, guar gum and xanthan gum is optionally included. One or more of citric acid, fructooligosaccharides, glycoproteins, bioflavonoids, polyglyceryl-10 oleate, glucose, sodium benzoate, phenoxyethanol, and propanediol is included in some embodiments.

The formulations may be formulated as a spray, such as a topical serum spray or a mist spray. The formulation, in several aspects, is configured for stimulating a vitamin D based compound (e.g., vitamin D and/or a precursor of vitamin D (e.g., lumisterol)). The stimulation includes, for example, an increase in at least one of vitamin D or vitamin D precursor storage, production, release, and activity. Such stimulation may occur in selective cells, such as one or both of keratinocytes and fibroblasts, or other cells. Optionally, stimulation can be advantageous in some embodiments because stimulation (such as release from storage) can occur even in low light or dark conditions. In one embodiment, vitamin D benefits are provided substantially continuously throughout a 24 hour period. The formulations function, according to some embodiments, to improve the viability or growth of beneficial skin microorganisms and reduce the viability or growth of harmful skin microorganisms.

In several embodiments, the formulations herein comprise one, two or all of the following: one or more prebiotics, one or more probiotics, and one or more postbiotics. In some embodiments, (i) the prebiotic comprises or consists essentially of a goji extract and/or a mushroom extract, (ii) the probiotic comprises or consists essentially of a Lactobacillus ferment and/or a Bifida ferment, and (iii) the postbiotic comprises or consists essentially of ferments that do not have activity but still allow beneficial microbes to thrive (such as squalane and other ferments). In one embodiment, a fermentation product of Lactobacillus, Bifida, beneficial yeast, etc. can be both a probiotic (as defined herein) and a postbiotic because they serve dual purposes of both repopulating beneficial microbes and creating an environment where those beneficial microbes can thrive.

In some embodiments, the formulations provided for herein enhance a user's skin care regimen by supporting skin barrier health and a balanced microbiome. In some embodiments, the formulations enhance skin's natural radiance for softer, smoother, and a more youthful-looking appearance. In some embodiments, the formulation is a cleanser (such as a 1-step cleanser). In some embodiments, the formulation is a moisturizer. In some embodiments, the cleanser and moisturizer are used sequentially. In several embodiments, a spray (such as a mist or a serum spray) is provided. Several embodiments stimulate vitamin D production and/or activity. In some embodiments, vitamin D is stimulated (e.g., stored, synthesized, activated, released and/or otherwise enhanced) in keratinocytes and fibroblasts (and in one embodiment, selectively in one or both of keratinocytes and fibroblasts). In some embodiments, the formulation(s) prepares skin for application of other skin care treatments and/or sunscreen in the day, protects and restores skin's barrier, and improves its natural resilience, while leaving skin refreshed and reset. In some embodiments the formulation works at multiple levels of the skin to hydrate, smooth and soften rough texture, and/or plump appearance of fine lines. In some embodiments, the formulation boosts overall skin health consistently for healthier, more resilient and smoother skin. In some embodiments, the formulation helps to enhance skin's natural resilience and radiance for softer, smoother, stronger skin and a more youthful, healthier-looking appearance. In some embodiments, the formulation balances flora and reduces dysbiosis to achieve a healthy microbiome. Flora or microbes on the skin include some mix of bacteria, viruses and/or fungi. In some embodiments, the formulation regulates the acid-mantle, a film-like covering on the skin that is made up of seat and sebum that maintains an acidic-skin surface pH between 4.7-5.6 and helps balance the skin microbiome. In some embodiments, the formulation restores the lipid barrier in skin, which is a complex moisture of lipids (e.g., fats), proteins and other molecules. In some embodiments, the formulation renews epidermal layer of skin.

In some embodiments, the moisturizer provided herein replenishes hydration and helps support improved skin health and appearance. In some embodiments, the advanced antioxidants and botanicals in the moisturizer provide soothing defense and improve skin's resilience to daily environmental exposure.

In some embodiments, the cleanser (e.g., a 1-step cleanser) is formulated to remove makeup and sunscreen in a single step. In some embodiments, the cleanser rinses the skin clean without leaving skin feeling stripped.

In some embodiments, several of the ingredients work synergistically. The synergism achieves a beneficial result that is more efficacious than the additive effects of the ingredients, according to some embodiments.

In several embodiments, there is provided a cosmetic formulation for moisturizing skin, the formulation comprising (a) ingredients to provide multi-level hydration, (b) ingredients to provide barrier defense, and (c) ingredients to soothe and protect. In several embodiments, the ingredients to provide multi-level hydration comprise one, two, three, or more of glycerin, apple extract, water, Tremella fuciformis sprocarp extract, betaine, squalane, sodium hyaluronate, wherein said sodium hyaluronate optionally provides multiple molecular weights. In several embodiments, the ingredients to provide barrier defense comprise one, two, three, or more of water, caprylic/capric triglyceride, cetyl palmitate, sorbitan stearate, polysorbate 80, hydrogenated lecithin, palmitoyl tetrapeptide-10, sodium benzoate, tocopherol, citric acid, propanediol, and Lycium barbarum (Goji) fruit extract. In several embodiments, the ingredients to soothe and protect comprise one, two three, or more of Opuntia ficus-indica stem extract, glycerin, phenoxyethanol, disodium lauriminodipropionate tocopheyl phosphates, benzoic acid, dehydroacetic acid, water, hydrolyzed pea protein, glucose, sodium chloride, and sodium succinate.

In several embodiments, there is also provided a cosmetic formulation for cleansing skin, the formulation comprising ingredients to remove impurities from the skin, replenish the skin, and rebalance the skin. In several embodiments, the ingredients comprise a plurality of ingredient selected from Opuntia ficus-indica stem extract, Citrus paradisi (Grapefruit) fruit extract, Lactobacillus ferment, sodium lauroyl sarcosinate, cocamidopropyl hydroxysultaine. In several embodiments, the cleansing formulation further comprises one or more of the following: citric acid, cocamidopropyl betaine, cocamidopropylamine oxide, decyl glucoside, glycereth-18, glycereth-18 ethylhexanoate, glycerin, methyl glucose caprate/caprylate/oleate, phenoxyethanol, potassium sorbate, propanediol, sodium benzoate, sodium chloride, sorbeth-230 tetraoleate, sorbitan laurate, trisodium ethylenediamine disuccinate, and water.

In several embodiments, there is also provided a cosmetic formulation for repairing and maintaining the skin barrier, said formulation comprising at least one prebiotic, at least one probiotic, at least one postbiotic, and ingredients selected from one or more of the following: apple extract, glycerin prickly pear extract, sodium hyaluronate, hydrolyzed pea protein. In several embodiments, the at least one prebiotic comprises one or both of a Tremella fuciformis sprocarp extract and a Lycium barbarum (Goji) fruit extract. In several embodiments, the at least one probiotic comprises Lactobacillus or Bifida bacterial ferments or yeast ferments, extracts, lysates, ferment lysates, isolates, etc. (which may or may not include the living microbes) that repopulate beneficial microbes. In several embodiments, the at least one postbiotic comprises squalane and/or other ferments that provide an environment for the beneficial microbes to thrive, which in turn supports a healthy biodiversity on the skin. In one embodiment, a formulation adapted for acne-prone skin is provided and further includes one or more of the following ingredients' alpha-hydroxy acids (AHAs) and beta-hydroxy acids (BHAs). In one embodiment, a formulation adapted for acne-prone skin is provided and further includes one or more of the following ingredients: salicylic acid, lactic acid, glycolic acid, sulfur, benzoyl peroxide, azelaic acid, niacinamide, retinoids (such as retinal, retinol, adapalene), tranexamic acid (e.g., cetyl tranexamate mesylate), alpha arbutin, and tea tree oil. In several embodiments, these additional ingredients are each provided in a range of about 0.05-10% of the total formulation (e.g., 0.05-1%, 0.5-1%, 1-2%, 2-3%, 3-5%, 5-10%, and overlapping ranges therein). In one embodiment, an effective amount of an ingredient to treat acne is provided that is at least 1% of the formulation (e.g., 1-10%, 1-15%, or 1-20%). One or more of these ingredients may be included in formulations to increase skin brightening in addition to or instead of being adapted for acne-prone skin.

Many embodiments achieve at least one of the following benefits (and in some embodiments, all of the benefits): (i) cleanses and/or moisturizes the skin, (ii) reduces build-up of dirt, oil, and/or makeup, (iii) softens and/or plumps the skin, (iii) trains the skin to become more resistant to blue light and/or UV light (e.g., to reduce sun damage, optionally in the absence of a specific Sun Protection Factor), (iv) maintains and/or enhances the skin microbiome using prebiotics, probiotics, and/or postbiotics, (v) reduces infection and/or inflammation; and (vi) regulates the acid-mantle.

In some embodiments, hydration and/or heathy microbes are increased by 25-75% or more post treatment with the formulations described herein (within minutes, hours or days post treatment). In some embodiments, the hydration level of the skin and/or healthy microbes are increased by at least 25-95% post treatment with formulations provided for herein used consistently (e.g., 1, 2 or 3 times daily) after, for example, 7, 14, 21, 28, 60 or 90 days. Healthy microbes in one embodiment are healthy bacteria, including but not limited to one or more of the following Lactobacillus sp. and Bifidobacterium sp. In one embodiment, undesired microbes are reduced on the skin by 10-90% after using the formulations described herein. Unhealthy microbes include but are not limited to undesired bacteria and fungi (such as yeast for example). Undesired bacteria include but are not limited to overgrowth of Cutibacterium acnes (Propionibacterium acnes), Corynebacterium granulosum, Propionibacterium granulosum, Group A Streptococcus and Staphylococcus aureus. Unhealthy microbes include in some embodiments Candida albicans, Tinea faciei and other dermatophytes. Balancing the microbiome or controlling dysbiosis reduces bacterial and/or other microbial toxins in some embodiments, which then leads to healthier skin (e.g., by reducing toxin burden, reducing inflammation, etc.)

In some embodiments, the formulations provided herein are concentrated and provided in a mask form to be left on the skin for at least 5, 15, 30, or 60 minutes (or as an overnight repair mask). Gentle cleansers may also include many of the ingredients of the formulations described herein and may additionally include micellar water, witch hazel and/or or other gentle toners (for toner-type cleansers) or foaming ingredients (for washes and foaming cleansers). Cleansing oils are provided in some embodiments, including but not limited to eye makeup remover. In several embodiments, more concentrated formulations are provided for use as a spot treatment or for treatment of an area smaller than the area treated by a mask.

In several embodiments, the formulations are provided as a liquid, cream, gel, toner, or essence, which includes for example serums and ointments. Powders and sprays (such as mists), and other topical formulations, are also provided. In some embodiment, the formulation is a mineral-based formulation. The formulations are vegan and cruelty-free in several embodiments. The formulations may be free of parabens and/or animal products. In one embodiment, the formulations decrease inflammatory mediators. The formulations may increase skin hydration and/or improve barrier functions of the epidermal layer.

In some embodiments, the formulation is a moisturizer and comprises Tremella fuciformis sporocarp extract, Opuntia ficus-indica stem extract, Lactobacillus ferment, squalane, Lycium barbarum (Goji) fruit extract, Pyrus malus (Apple) fruit extract and Sodium Hyaluronate. In some embodiments, the formulation is a cleanser and comprises Opuntia ficus-indica stem extract, Lactobacillus ferment, and Citrus paradisi (Grapefruit) fruit extract.

Additionally, there is provided for herein a cosmetic formulation for moisturizing skin, comprising ingredients to provide multi-level hydration, ingredients to provide barrier defense, and ingredients to soothe and protect.

In several embodiments, the ingredients to provide multi-level hydration comprise glycerin, apple extract, cactus extract (e.g., Opuntia streptacantha and/or Opuntia ficus-indica extract) water, Tremella fuciformis sporocarp extract, betaine, squalane, and/or sodium hyaluronate. In several embodiments, the sodium hyaluronate is optionally provide multiple molecular weights.

In several embodiments, the ingredients to provide barrier defense comprise water, caprylic/capric triglyceride, cetyl palmitate, sorbitan stearate, polysorbate 80, hydrogenated lecithin, palmitoyl tetrapeptide-10, sodium benzoate, tocopherol, citric acid, propanediol, Lactobacillus ferment, bifida ferment, and/or Lycium barbarum (Goji) fruit extract Peptides, natural or synthetic (or a combination of both) are used instead of palmitoyl tetrapeptide-10 in several embodiments.

In several embodiments, the ingredients to soothe and protect comprise Opuntia ficus-indica stem extract, glycerin, phenoxyethanol, disodium lauriminodipropionate tocopheryl phosphates, benzoic acid, dehydroacetic acid, water, hydrolyzed pea protein, glucose, sodium chloride, and/or sodium succinate.

In several embodiments, the ingredients to soothe and protect comprise one or more vitamin D based compounds, including vitamin D derivatives, analogues, pro vitamin D, e.g. cholecalciferol, calcitriol, calcipotriene 7-Dehydrocholesterol (7-DHC), vitamin D2, vitamin D3, etc. The vitamin D based compound comprises lumisterol in one embodiment. In one embodiment, the amount is provided in the range of 0.01%-10% of the formulation or 250-10,000 IU (e.g., 250-2500, 2500-7500, 7500-10,000 IU) per application. Instead of, or in addition to, vitamin D based compounds, several embodiments of the formulations stimulate a vitamin D based compound (e.g., at least one of vitamin D and a precursor of vitamin D).

In several embodiments, there is provided a cosmetic formulation for cleansing skin, comprising ingredients to remove impurities from the skin, replenish the skin and rebalance the skin. In several embodiments, the ingredients comprise two, three, four or more of Opuntia ficus-indica stem extract, Opuntia streptacantha extract, Citrus paradisi (Grapefruit) fruit extract, Lactobacillus ferment, sodium lauroyl sarcosinate, and cocamidopropyl hydroxysultaine. Additionally included, in several embodiments, is one or more of citric acid, cocamidopropyl betaine, cocamidopropylamine oxide, decyl glucoside, glycereth-18, glycereth-18 ethylhexanoate, glycerin, methyl glucose caprate/caprylate/oleate, phenoxyethanol, potassium sorbate, propanediol, sodium benzoate, sodium chloride, sorbeth-230 tetraoleate, sorbitan laurate, trisodium ethylenediamine disuccinate, and water.

In several embodiments, there is also provided a cosmetic formulation for repairing and maintaining the skin barrier, said formulation comprising at least one prebiotic, wherein the at least one prebiotic comprises one or both of a Tremella fuciformis sporocarp extract and a Lycium barbarum (Goji) fruit extract, at least one probiotic, wherein the at least one probiotic comprises a bacterial fermentation product selected from one or both of the following: Lactobacillus ferment and Bifida ferment, at least one postbiotic, wherein the at least one postbiotic comprises a yeast fermentation product, wherein said yeast fermentation product comprises squalane. In several embodiments, the formulation for repairing and maintaining the skin barrier further comprises ingredients selected from one or more of apple extract, glycerin, prickly pear extract, sodium hyaluronate, hydrolyzed pea protein.

In several embodiments, there is provided a cosmetic formulation comprising effective amounts of at least one skin hydrating agent, at least one agent to improve skin barrier function, at least one prebiotic agent to improve skin microbiome health, at least one probiotic and postbiotic selected from the group consisting or consisting essentially of Lactobacillus ferment, Bifida ferment, and yeast ferment, and at least one preservative, wherein the preservative is natural or synthetic.

In several embodiments, the at least one skin hydrating agent comprises one or more of Pyrus malus (Apple) fruit extract, glycerin, and Tremella fuciformis sporocarp extract. In several embodiments, the at least one agent to improve skin barrier function comprises one or both of caprylic/capric triglyceride and cetyl palmitate. In several embodiments, the at least one prebiotic agent comprises Lycium barbarum (Goji) fruit extract and/or Tremella fuciformis sporocarp extract.

In several embodiments, the formulation is configured for topical use and wherein the yeast ferment is squalane. In several embodiments, the at least one skin hydrating agent is present in an amount between 0.005% and 10% (e.g., 0.01% and 8%) of the formulation. In several embodiments, the at least one skin hydrating agent comprises Pyrus malus (Apple) fruit extract and is present in an amount between 0.01% and 9% (e.g., 0.1% and 5%) of the formulation. In several embodiments, the at least one agent to improve skin barrier function is present in an amount between 0.01% and 9% (e.g., 0.5% and 6%) of the formulation. In several embodiments, the at least one prebiotic agent to improve skin microbiome health is present in an amount between 0.001% and 10% (e.g., 0.005% and 6%) of the formulation. In several embodiments, the at least one probiotic agent to improve skin microbiome health is present in an amount between 0.01% and 10% (e.g., 0.05% and 5%) of the formulation. In several embodiments, the at least one probiotic agent comprises a Lactobacillus ferment and/or a Bifida ferment. In several embodiments, the at least one postbiotic agent is present in an amount between 0.05% and 7% (e.g., 0.1% and 4%) of the formulation, and wherein the formulation functions to improve the viability or growth of beneficial skin microorganisms and reduce the viability or growth of harmful skin microorganisms. In one embodiment, vitamin D is stimulated (e.g., stored, synthesized, activated, released and/or otherwise enhanced).

In several embodiments, the formulation further comprises one or more of Opuntia ficus-indica extract, Opuntia streptacantha extract, hydrolyzed pea protein, betaine, and disodium lauriminodipropionate tocopheryl phosphates. In several embodiments, the formulation further comprises an antioxidant, wherein the antioxidant quenches free radicals.

In several embodiments, there is provided a cosmetic formulation comprising effective amounts of caprylic/capric triglyceride, cetyl palmitate, sorbitan stearate, polysorbate 80, hydrogenated lecithin, palmitoyl tetrapeptide-10, Lycium barbarum (Goji) fruit extract, Tremella fuciformis sporocarp extract, Lactobacillus ferment, and a preservative. Peptides, natural or synthetic (or a combination of both) are used instead of palmitoyl tetrapeptide-10 in several embodiments.

In several embodiments, the preservative comprises phenoxyethanol and/or an additional broad spectrum preservative. In several embodiments, the formulation further comprises at least one of tribehenin and squalane. In several embodiments, the formulation further comprises one or more of Opuntia ficus-indica extract, Opuntia streptacantha extract, pea protein (e.g., hydrolyzed pea protein), and glyceryl caprylate. In several embodiments, the effective amount of the Tremella fuciformis sporocarp extract is provided in a range of 0.0001% to 4% (e.g., 0.001-2%) of the formulation. In several embodiments, the effective amount of the Lycium barbarum (Goji) fruit extract is provided in a range of 0.0001% to 4% (e.g., 0.001-2%) of the formulation. In several embodiments, the cosmetic formulation further comprises at least one ingredient to hydrate skin, which is, in several embodiments, selected from one or more of glycerin, betaine, and sodium hyaluronate (optionally provided in multiple molecular weights).

In several embodiments, there is provided a topical cleansing formulation comprising effective amounts of at least one surfactant, at least one humectant, Citrus paradisi (Grapefruit) fruit extract, at least one agent to improve skin microbiome health, and at least one preservative, wherein the preservative is natural or synthetic. In several embodiments, the at least one surfactant comprises one or more of sodium lauroyl sarcosinate and cocamidopropyl hydroxysultaine. In several embodiments, the at least one humectant is present in an amount between 0.01% and 10% (e.g., 0.5% to 5%) of the formulation. In several embodiments, the at least one agent to improve skin microbiome health comprises a Lactobacillus ferment and/or a Bifida ferment. In several embodiments, the formulation further comprises Opuntia ficus-indica extract (e.g., Opuntia ficus-indica stem extract). In several embodiments, the at least one agent to improve skin microbiome health comprises a ferment from gram-positive, lactic acid producing bacteria. In several embodiments, the at least one surfactant comprises sodium lauroyl sarcosinate and the sodium lauroyl sarcosinate is present in an amount between 3% and 15% (e.g., 6% and 12%) of the formulation. In several embodiments, the at least one surfactant comprises cocamidopropyl hydroxysultaine, and the cocamidopropyl hydroxysultaine is present in an amount between 1% and 10% (e.g., 2% and 8%) of the formulation. In several embodiments, the at least one surfactant comprises both sodium lauroyl sarcosinate and cocamidopropyl hydroxysultaine. In several embodiments, the Citrus paradisi (Grapefruit) fruit extract is present in an amount between 0.001% and 5% (e.g., 0.01% and 3%) of the formulation. In several embodiments, the Lactobacillus ferment and/or Bifida ferment is present in an amount between 0.01% and 6% (e.g., 0.1 and 3%) of the formulation. In several embodiments, the topical cleansing formulation further comprises one or more skin conditioning agent selected from cocamidopropyl hydroxysultaine, cocamidopropylamine oxide, methyl glucose caprate/caprylate/oleate, glycereth-18, and glycereth-18 ethylhexanoate. In several embodiments, the Citrus paradisi (Grapefruit) fruit extract promotes gentle exfoliation. In several embodiments, the topical cleansing formulation is configured to remove makeup and mineral sunscreen from the skin of a user. In several embodiments, the formulation advantageously allows removal of makeup and/or sunscreen from the skin of a user in a single step.

In several embodiments, there is provided a cosmetic formulation for repairing the skin barrier, said formulation comprising at least one prebiotic, at least one probiotic and postbiotic selected from the group consisting or consisting essentially of Lactobacillus ferment, Bifida ferment, and yeast ferment, and one or more of apple extract, glycerin, prickly pear extract, sodium hyaluronate, hydrolyzed pea protein, and preservatives. In one optional aspect, vitamin D is stimulated (e.g., stored, synthesized, activated, released and/or otherwise enhanced).

In several embodiments, there is provided a cosmetic formulation for hydrating the skin. In some embodiments, the cosmetic formulation comprises effective amounts of at least one surfactant, at least one humectant, at least one agent to improve skin microbiome health, and at least one preservative, wherein the preservative is natural or synthetic. In several embodiments, the at least one surfactant comprises polyglyceryl-10 oleate. In several embodiments, the at least one humectant is present in an amount between 0.001% and 10% (e.g., 0.1% to 5%) of the formulation. In several embodiments, the at least one agent to improve skin microbiome health comprises a Lactobacillus ferment and/or a Bifida ferment. In several embodiments, the formulation further comprises Opuntia ficus-indica extract (e.g., Opuntia ficus-indica stem extract). In several embodiments, the at least one agent to improve skin microbiome health comprises a ferment from gram-positive, lactic acid producing bacteria. In several embodiments, the at least one surfactant polyglyceryl-10 oleate is present in an amount between 0.5% and 7% (e.g., 1% and 5%) of the formulation. In several embodiments, cosmetic formulation comprises an anti-aging agent. In some embodiments, an anti-gaing agent such as glutamylamidoethyl imidazole is present in an amount between 0.0001% and 5% (e.g., 0.001% and 1%) of the formulation. In several embodiments, the Lactobacillus ferment and/or Bifida ferment is present in an amount between 0.01% and 6% (e.g., 0.1 and 3%) of the formulation. In several embodiments, the cosmetic formulation further comprises one or more skin conditioning agents selected from gluconolactone, sodium hyaluronate, polyglyceryl-10 oleate, pentylene glycol, fructooligosaccharides, glycoproteins and bioflavonoids. In several embodiments, the one or more skin conditioning agents are present in an amount between 0.01 to 5% (e.g., 0.01% and 3%) of the formulation. In several embodiments, the cosmetic formulation is configured to be used as a first layer on a cleansed skin. In several embodiments, the formulation is used to provide hydration for skin throughout the day. In several embodiments, the formulation is used to provide hydration for skin in the night. In several embodiments, the formulation reduces the appearance of wrinkles on the skin. In several embodiments, the formulation reduces the appearance of wrinkles on the skin by inhibiting metalloproteinases (MMPS) like collagenase-1 on the skin, In several embodiments, the formulation increases skin tonicity. In several embodiments, the formulation further comprises one or more of hydrolyzed pea protein and Cyamopsis Tetragonoloba (Guar) Gum. In several embodiments, the formulation further comprises an antioxidant, wherein the antioxidant quenches free radicals. In several embodiments, the formulation stimulates vitamin D synthesis and/or the synthesis of vitamin D precursors in the skin. Several embodiments stimulate vitamin D production and/or activity. In some embodiments, vitamin D is stimulated (e.g., stored, synthesized, activated, released and/or otherwise enhanced) in only or at least in keratinocytes and fibroblasts. Several embodiments increase at least one precursor to vitamin D (such as lumisterol), which in turn may act as a vitamin D reservoir allowing for vitamin D metabolites to be activated. In some embodiments, the formulation stimulates vitamin D even in low light or dark conditions. In one embodiment, the precursors are selectively increased in fibroblasts or other cells. In some embodiments, the formulation stimulates vitamin D and/or precursors in cutaneous cells. In some embodiments, the formulation stimulates vitamin D and/or precursors in epidermis cells. The storage of vitamin D and/or precursors may be helpful to provide sustained release.

In several embodiments, the formulation stimulates cutaneous circadian genes (e.g., CLOCK, BMAL1, PER1, PER2 etc.) that are involved in regeneration and repair of skin cells. In some embodiments, the formulation regulates the circadian biorhythm that regulates skin's ability to repair DNA damage, reduce oxidative damage, reduce inflammation and synthesize collagen. In some embodiments, regulation of circadian biorhythm by the formulation disclosed herein prevents premature aging of the skin.

In several embodiments, there is provided a method of moisturizing skin, comprising applying a formulation, or instructing the application of a formulation(s) of the present disclosure to skin to be moisturized. In several embodiments, there is provided a method of cleansing skin, comprising applying a formulation, or instructing the application of a formulation(s), of the present disclosure to skin to be cleansed. In several embodiments, the skin is dry. In several embodiments, the skin is oily. In several embodiments, regions of the skin are dry, while other regions of the skin are oily. In several embodiments, the skin has wrinkles. In several embodiments, the skin has inflammation. In several embodiments, the skin has blemishes. In several embodiments, the skin is sun damaged. In several embodiments, the skin is healthy.

In several embodiments, there is provided use of a formulation according to the present disclosure for moisturizing (e.g., hydrating) and/or cleansing the skin. In several embodiments, there is provided use of a formulation according to the present disclosure to repair or maintain the acid mantle of the skin and/or reduce skin dysbiosis. In several embodiments, the formulation is used to treat the skin, and optionally acne, eczema, psoriasis, dermatitis, and/or rosacea.

In several embodiments, there is provided a kit comprising a formulation(s) according to the present disclosure instructing the application of a formulation to skin to be moisturized. In several embodiments, there is provided a kit comprising the formulation(s) according to the present disclosure instructing the application of a formulation to skin to be cleansed.

In several embodiments, there is provided a method for moisturizing a region of skin of a user comprising applying a formulation, or instructing the application of a formulation to the region of skin, the formulation comprising at least one skin hydrating agent, at least one agent to improve skin barrier function, at least one prebiotic agent to improve skin microbiome health, at least one probiotic and postbiotic selected from Lactobacillus ferment, Bifida ferment, and yeast ferment; and at least one preservative, wherein the preservative is natural or synthetic.

In several embodiments, the at least one skin hydrating agent comprises one or more of Pyrus malus (Apple) fruit extract, glycerin, and Tremella fuciformis sporocarp extract. In several embodiments, the at least one agent to improve skin barrier function comprises one or both of caprylic/capric triglyceride and cetyl palmitate. In several embodiments, the at least one prebiotic agent comprises Lycium barbarum (Goji) fruit extract and/or Tremella fuciformis sporocarp extract.

In several embodiments, there is provided a method for cleansing a region of skin of a user comprising applying a formulation, or instructing the application of a formulation to the region of skin, the formulation comprising at least one surfactant, at least one humectant, Citrus paradisi (Grapefruit) fruit extract, at least one probiotic and postbiotic selected from Lactobacillus ferment, Bifida ferment, and yeast ferment, and at least one preservative, wherein the preservative is natural or synthetic. In several embodiments, the at least one surfactant comprises one or more of sodium lauroyl sarcosinate and cocamidopropyl hydroxysultaine. In several embodiments, the at least one humectant is present in an amount between 0.1% and 10% (e.g., 0.5% to 5%) of the formulation.

In several embodiments, there is provided a method for treating a region of skin of a user comprising applying a formulation, or instructing the application of a formulation to the region of skin, the formulation comprising at least one prebiotic, at least one probiotic and postbiotic selected from Lactobacillus ferment, Bifida ferment, and yeast ferment, and, optionally, at least one ingredient to hydrate skin. In several embodiments, the skin is healthy or wherein the skin has one or more of winkles, inflammation, blemishes, and/or sun damage.

In several embodiments, there is provided the use of formulation for treating skin or for the preparation of a skin treatment, the formulation comprising at least one prebiotic, at least one probiotic, at least one postbiotic, and, optionally, at least one ingredient to hydrate skin.

Also provided for herein is a kit comprising two formulations for treatment of skin, the kit comprising two or more of a first formulation for cleansing a region of skin, the first formulation comprising at least one surfactant, at least one probiotic and postbiotic selected from Lactobacillus ferment and Bifida ferment, and, optionally, at least one ingredient to hydrate skin and a second formulation for moisturizing the region of skin, the second formulation comprising at least one prebiotic, at least one probiotic and postbiotic selected from the group consisting or consisting essentially of Lactobacillus ferment, Bifida ferment, and yeast ferment, and at least one ingredient to hydrate skin, and another formulation for treating the skin. In several embodiments, the kit also includes an applicator for applying one or more the formulations. In several embodiments, the kit also includes a brush-on sunscreen. In several embodiments, the first formulation further comprises Citrus paradisi (Grapefruit) fruit extract. In several embodiments, the at least one prebiotic in the second formulation comprises Lycium barbarum (Goji) fruit extract and/or Tremella fuciformis sporocarp extract. In several embodiments, the at least one skin hydrating agent is selected from one or more of Pyrus malus (Apple) fruit extract, glycerin, and Tremella fuciformis sporocarp extract. In several embodiments, the kit also includes instructions for use.

Additionally, there is also provided a formulation, method, use or kit according to the present disclosure that provides protection against damaging light, such as blue light, without using titanium dioxide, zinc oxide, oxybenzone, avobenzone, homosalate, octinoxate, octocrylene, or octisalate. In several embodiments, the formulations, methods, uses or kits according to the present disclosure allows an increase in the skin's own ability to resist damaging light, such as high-energy visible light (e.g., blue light). In several embodiments, the formulations, methods, uses or kits according to the present disclosure reduces the number and/or activity of Propionibacterium acnes (aka Cutibacterium acnes). In several embodiments, the formulations, methods, uses or kits according to the present disclosure increase and/or maintain the number and/or activity of beneficial skin flora and/or reduce dysbiosis.

### DETAILED DESCRIPTION

Several embodiments described herein are formulations that provide hydration, protect against damaging light (such as blue light) and contribute to a healthy skin microbiome. In some embodiments, prebiotics, probiotics and postbiotics are included in the formulation.

Both healthy and damaged skin can benefit from several of the formulations described herein. Damaged skin can include skin with hyperpigmentation, UV damage (including, but not limited to, sunburned skin, fine lines, wrinkles, enlarged pores and/or skin laxity), sensitive skin, dry skin, oily skin, acne and/or rosacea.

An enhanced glow or radiance is achieved by several embodiments. Many formulations herein are not only therapeutic and protective, but also nourish the skin, increase hydration, balance the microbiome, and improve overall appearance, texture and firmness. Thus, several formulations of the invention are beneficial for skin that is aged, sun-damaged, wrinkled, lax, and/or blemished. Formulations according to several embodiments can be used anywhere on the body, and are especially beneficial for the face, neck, decolletage and hands, where signs of skin damage and aging may be particularly prominent.

In one embodiment, the formulation is configured for replenishing the skin. In other embodiments, the formulation can be used to rebalance the skin microbiome the skin. In several embodiments, the formulation soothes the skin. In several embodiments, the formulation provides hydrating and/or moisturizing effects. In several embodiments, the formulation provides skin firmness. In several embodiments, the formulation has antiaging benefits. In several embodiments, the formulation stimulates the synthesis and/or activity of vitamin D and/or a precursor of vitamin D, upon exposure to light. In several embodiments, the formulation mimics light exposure to activate cellular mechanisms that prevent premature aging of the skin. In several embodiments, the formulation promotes and/or maintains a balanced skin microbiome to support a healthy skin barrier. In several embodiments, the formulation provides hydrating and/or moisturizing effects without feeling heavy on the skin. In several embodiments, the formulation works at multiple levels of the skin to hydrate, smooth and soften rough texture, and plump appearance of fine lines. In some embodiments, the formulation provides anti-inflammatory properties. In some embodiments, the formulation contains antioxidants to neutralize free radical damage and improve skin's resilience to environmental aggressors including, but not limited to, blue (HEV) light and ultraviolet light (e.g., UVA and/or UVB). In some embodiments, the formulations function as a sunscreen, but without an SPF such as a chemical or physical SPF ingredient (such as titanium dioxide, zinc oxide, oxybenzone, avobenzone, homosalate, octinoxate, octocrylene, octisalate, etc.). In some embodiments, the formulation is oil-free. In some embodiments, the formulation is fragrance-free. In one embodiment, the formulation additionally replenishes, restores, repairs, soothes and/or protects the skin in a multi-modular manner.

In some embodiments, the invention comprises a topical formulation for treating dry, flaky, and/or itchy skin in a subject, wherein the topical formulation is a cream, liquid (such as serum, lotion, oil, mist, toner, or essence), a gel, a spray, a powder, a jelly, or a combination thereof.

The use of the terms agents, ingredients and compounds may be used interchangeably herein.

In some embodiments, the formulation is a moisturizer and comprises one, two, three or more of the following ingredients: Tremella fuciformis sporocarp or other mushroom extract, Opuntia ficus-indica stem or other cactus extract (such as prickly pear) extract, Lactobacillus ferment or other healthy bacteria or bacterial product, Lycium barbarum (goji) fruit or other lyceum extract, and Pyrus malus (apple) fruit or other Pyrus extract.

In some embodiments, the formulation is a cleanser and comprises one, two, three or more of the following ingredients: Opuntia ficus-indica stem or other cactus extract (such as prickly pear) extract, Lactobacillus ferment or other healthy bacteria or bacterial product, Citrus paradisi(Grapefruit) fruit extract, as well as one or more surfactants such as cocamidopropyl hydroxysultaine, and sodium lauroyl sarcosinate.

In several embodiments, a mist is provided, wherein the mist comprises one, two, three or more of the following ingredients: Opuntia ficus-indica stem or other cactus extract (such as prickly pear) extract, Lactobacillus ferment or other healthy bacteria or bacterial product, Lycium barbarum (goji) fruit or other lyceum and hydrolyzed Pea Protein. The mist may also include one or more of sodium hyaluronate, glycerin, squalane, tocopherol, phenoxyethanol and ethylhexylglycerin. In some embodiments, the mist is provided as a spray-on mist to be used on clean skin. In other embodiments, the mist can also be used to refresh the skin during the day and on top of make up or sunscreen. In some embodiments, the mist can be setting spray. In some embodiments, the mist may also be used to remove make up or sunscreen and applied directly to the skin and wiped off, or be applied to a cotton pad or other applicator. In some embodiments, the formulation for a mist is configured as a liquid toner which can be applied with an applicator such as a cotton pad, and be used for make up or sunscreen removal. In some embodiments, these ingredients may also be used in the form of a serum.

In several embodiments, a formulation (e.g., spray) is provided, wherein the formulation comprises one, two, three or more of the following ingredients: Opuntia ficus-indica stem or other cactus extract (such as prickly pear) extract, Lactobacillus ferment or other healthy bacteria or bacterial product, Lycium barbarum (goji) fruit or other lyceum and hydrolyzed pea protein. The formulation may also include one or more of sodium hyaluronate, glycerin, squalane, tocopherol, phenoxyethanol, ethylhexylglycerin, glutamylamidoethyl imidazole, fructooligosaccharides, glycoproteins, and bioflavonoids. In other embodiments, the formulation can also be used to refresh the skin during the day and on top of make up or sunscreen. In some embodiments, the formulation may also be used under makeup or sunscreen and applied directly to the skin and wiped off, or be applied to a cotton pad or other applicator. In some embodiments, the formulation for a spray is configured as a liquid toner which can be applied with an applicator such as a cotton pad. The spray may be a fine mist or a thicker serum. In some embodiments, the spray can be applied on clean skin, followed by sunscreen, serum(s) and/or makeup.

In several embodiments, the formulation comprises amino acids, peptides or proteins, lithops, hyaluronic acid, prickly pear cactus extract, goji berry extract, lactobacillus ferment, squalane and hydrolyzed pea protein. In some embodiments, the lithops is lithops plant cell nectar and the hyaluronic acid is in trimolecular form. In some embodiments, a chronopeptide or chrono-compound such as glutamylamidoethyl imidazole activates circadian genes which mimic, enhance availability or stimulate a vitamin D compound, and is provided synergistically in combination with a fructooligosaccharide, a glycoprotein, and a bioflavonoid that may be extracted from succulents, including but not limited to lithops, to stimulate the production of a vitamin D based compound without the need for damaging UV light.

In several embodiments, the formulation comprises an Opuntia ficus-Indica stem extract, a Lycium barbarum fruit extract, a Lactobacillus ferment, glutamylamidoethyl imidazole, sodium hydroxide; and a synthetic preservative. The formulation may comprise one or more of the following: Opuntia Ficus-Indica stem extract in an amount between 0.001-2% of the formulation; Lycium barbarum fruit extract is present in an amount between 0.001-2% of the formulation; glutamylamidoethyl imidazole present in an amount between 0.001-2% of the formulation. The formulation may further comprise one or more of fructooligosaccharides, glycoproteins, bioflavonoids, polyglyceryl-10 oleate, glucose, sodium benzoate, citric acid, and propanediol.

In one embodiment, the formulation comprises or consists essentially of 80, 85, 90, 95% or 100% of the following ingredients: water, glycerin, polyglyceryl-10 oleate, squalane, lactobacillus ferment, sodium hydroxide, propanediol, fructooligosaccharides, glycoproteins, bioflavonoids, Lycium barbarum (goji) fruit extract, pentylene glycol, Opuntia ficus-indica stem extract, ethylhexylglycerin, calcium gluconate, gluconolactone, sodium hyaluronate, glutamylamidoethyl imidazole, Cyamopsis tetragonoloba (guar) gum, xanthan gum, glucose, hydrolyzed pea protein, phytic acid, sodium chloride, tocopherol, phenoxyethanol, sodium benzoate, and sodium succinate.

Bioflavonoids include but are not limited to, lithops extracted bioflavonoids, citrus bioflavonoids (e.g., naringin, neohesperidin, narirutin, and hesperidin), green tea extracts (e.g., Camellia sinensis leaf extract), catechin flavonoids, Calendula officinalis flower extracts, chamomile (Matricaria recutita flower extract), aloe barbadensis leaf extract, Brassica oleracea italica (broccoli) extract), anthocyanin, isorhamnetin, rutin, and terpene.

In some embodiments, betalain compounds are used instead of lithops or can be optionally be used as an extract from lithops.

The formulations provided herein may be provided as a serum (e.g., serum spray), cream, lotion, cleanser, mist, mask, patch, and powder. In several embodiments, rather than containing a vitamin D based compound, stimulates a vitamin D based compound. In another embodiment, a vitamin D based compound is contained in the formulation.

In several embodiments, formulations for stimulating a vitamin D based compound and enhancing the microbiome comprise a probiotic, a postbiotic, an ingredient to stimulate a vitamin D compound, and a synthetic preservative, wherein the formulation is provided as a topical formulation and is configured for use on a user's skin, wherein the formulation is configured for at least (i) stimulating vitamin D and/or a precursor of vitamin D, (ii) supporting the skin's microbiome, and/or (iii) restoring the skin's barrier. In some embodiments, the formulation comprises an Opuntia ficus-indica stem extract, a Lycium barbarum fruit extract, a Lactobacillus ferment, glutamylamidoethyl imidazole, sodium hydroxide, and a synthetic preservative, wherein the formulation is configured for at least (i) stimulating vitamin D and/or a precursor of vitamin D, (ii) supporting the skin's microbiome, and/or (iii) restoring the skin's barrier. The stimulation comprises an increase in at least one of storage, production, release, and activity of a vitamin D based compound.

In some embodiments, formulations for topical use for stimulating a vitamin D based compound comprise a plurality of skin conditioning agents comprising fructooligosaccharides, glycoproteins, bioflavonoids, glycerin, and a Opuntia Ficus-Indca extract, at least one probiotic, wherein the at least one probiotic comprises a Lactobacillus ferment, at least one postbiotic, wherein the at least one postbiotic comprises squalane; and one or more ingredients selected from the group consisting or consisting essentially of: a hydrolyzed pea protein, glutamylamidoethyl imidazole, polyglyceryl-10 oleate, sodium hydroxide, and pentylene glycol. The probiotic may comprises a Lactobacillus ferment (e.g., present in an amount between 0.001% and 3% of the formulation); and wherein the squalane may be present in an amount between 0.5% and 3% of the formulation. A synthetic preservative, glucose, guar gum and/or xanthan gum may be included. The formulation functions, in one embodiment, to improve the viability or growth of beneficial skin microorganisms and reduce the viability or growth of harmful skin microorganisms. Several embodiments increase beneficial skin flora and/or reduce dysbiosis. Antioxidants and/or anti-acne compounds are included in several embodiments. Improved skin barrier is achieved in one embodiment by maintaining a healthy skin microbiome. Excess sebum, acne, eczema, psoriasis, dermatitis, and/or rosacea may be treated.

In some embodiments, provided herein are a formulation, method, use and kit that (i) builds the skin's own ability to resist damaging light, such as high-energy visible light (e.g., blue light); (ii) reduces the number and/or activity of Propionibacterium acnes aka Cutibacterium acnes; and (iii) increase and/or maintains the number and/or activity of beneficial skin flora and/or reduce dysbiosis.

In some embodiments, the formulation is configured as a spray for stimulating a vitamin D based compound in at least one of keratinocytes and fibroblasts. The formulation stimulates a vitamin D based compound in low light and dark conditions (e.g., unexpectedly does not rely on light, such as damaging UV light) according to several embodiments. The formulation may selectively increase a vitamin D based compound in one or more cell types, where said cell types optionally comprise fibroblasts. The formulation is configured to increase desired bacterial strains and reduce undesired bacteria strains in one embodiment. For example, Propionibacterium acnes may be reduced. Beneficial effects occur upon use, within hours of use, within 1-2 days, 3-4 days, about 7 days, about 14 days, or within about 3 weeks, 2-4 weeks, about 4-6 weeks, about 6-10 weeks, or about 12-16 weeks of use.

In several embodiments, a formulation is provided to increase the bioavailability of a vitamin D based compound that comprises: (i) a chrono-compound that transforms, processes, or makes bioavailable a vitamin D compound (including but not limited to imidazoles, glutamic acid compounds, aminoethylimidazole, glutamylamidoethyl imidazole, and anti-glycation compounds, (ii) a fructooligosaccharide, (iii) a glycoprotein, and (iv) a bioflavonoid. Extracts from succulents may be used, including but not limited to lithops, to stimulate the production of a vitamin D based compound without the need for damaging UV light. Optionally, one or more of a pre, pro and postbiotic are included to support the skin's microbiome. Additional ingredients disclosed herein are used in some embodiments.

In several embodiments, use of the formulations described herein reduces skin damage (e.g., inflammation, irritation, dryness, imbalanced microbiome, oiliness, acne, laxity, premature lines, etc.) by about 10-100% (e.g., about 10%, 25%, 50%, 75%, 100% and ranges in between) after use (as compared to before application of the formulations described herein). For example, significant improvements in skin pore size (e.g., reductions in pore size and/or visibility), fine lines, overall appearance, radiance, skin smoothness, and/or skin tone (evenness), skin firmness and/or improved skin health (reduced itchiness, flaking etc.) are visible in several embodiments after 2 weeks, 4 weeks, 8 weeks or 12 weeks. In some embodiments, the formulation absorbs quickly into the skin. In some embodiments, the formulation absorbs into the skin after 1, 2, 3, 4, 5, 6, 7, 8, 10, 20, 40, 60, 120, 180 minutes. In some embodiments, the formulation absorbs into the skin after 1, 2, 3, 4, 5, 6, 7, 8 or 10 hours. Certain improvements in skin may be visible or felt within days of use. In several embodiments, the formulations provided for herein reduce and/or ameliorate one or more symptoms of irritation of skin (including itching or burning) and or reduce one or more symptoms of eczema, acne and/or rosacea.

In some embodiments of the invention, the anti-inflammatory agents decrease redness and irritation and fortify resistance to inflammation. The anti-inflammatory agents may have antioxidant properties by reacting with reactive oxygen species. The anti-inflammatory agents may also absorb the energy from IN light and are photo-protective and reduce UV-induced free radical damage to skin.

Although preservatives are provided in certain embodiments, the formulations described herein can be manufactured with reduced or no synthetic preservatives. In some embodiments, the formulations are free of one or more of the following: parabens, phthalates, sulfates, mineral oil, gluten, allergens, and irritants.

The agents, ingredients and compounds described herein may be modified natural substances (e.g., isolates, ferments, extracts, purified, processed, chemically modified, etc. and that for example may not occur naturally in nature) or synthetic substances. Methods of using unique combinations of natural substances are also provided.

In several embodiments, the invention comprises a method of treating skin using any one of the formulations described above or below. The use of any of the formulations described herein for treating skin, preventing skin damage, and/or improving skin appearance is provided in several embodiments. Several embodiments also include instructing the method or use of the formulation (e.g., via instructions for use).

The areas of the skin to be treated include, for example, the entire facial region, from under eye to brow, including eyelids, other areas of the head and neck. Although the formulations described herein are useful to treat the eye area, in some embodiments the formulations can also be used to treat other skin regions that would benefit from brightening, reduced puffiness, smoothing of fine lines and wrinkles, hydration and sun protection. For example, the nasolabial folds and marionette lines may be particularly well suited to benefit from the formulations described herein. Scars may be treated in some embodiments.

In several embodiments, the formulation is contained within an applicator with a dispensing tip. The applicator may have a metallic tip in some embodiments. The applicator may be pen or cylindrical shaped in one embodiment and the tip may be a round or oval partially flattened or curved shape to help application to the contours of the eye and other areas. For eye applications, the tip may be narrow and contoured to navigate the contours of the eye region. Larger applicators may be used for other regions on the face and body. In several embodiments, the formulation is used during or after a dermatological procedure (including but not limited to brow lifts, blepharoplasty, botulinum and other neurotoxins, facials, fillers, dermabrasion, microdermabrasion, micro-needling, peels, exfoliations, suctioning, fluid delivery, acid treatments, massage, extractions, energy-based and other treatments, such as lasers, thermal, radiofrequency, light (e.g., redlight therapy, photofacials, IPL), etc.). In some embodiments, the applicator includes fluid delivery conduits, pressure/air-based devices, and sonicators. In several embodiments, formulations may be stored and/or delivered by use of a bottle and/or dropper. In several embodiments, the dropper or other delivery device is graduated and/or indexed to deliver pre-set amounts of a formulation disclosed herein. In some embodiments, the applicator includes a paddle or contoured metallic tip to facilitate application.

In some embodiments, the formulation is contained within an applicator having a tip with a thermal conductivity configured to provide a cooling effect. In several embodiments, the invention comprises a use or method of increasing the hydration of the skin, improving health of the skin microbiome, blue and/or IV light protection, and reduced inflammation and/or infection of the skin, comprising administration, or instructing administration of a formula described herein. A kit is provided in some embodiments, wherein the kit is configured for increasing the hydration of the skin, improving health of the skin microbiome, blue and/or UV light protection, and reduced inflammation and/or infection of the skin (and additionally in some embodiments, reduces laxity, puffiness, wrinkles and/or dark circles around the eye) and includes a formulation as described herein, wherein the formulation is optionally contained within an applicator having a metallic cooling tip.

The formulations described herein can provide both short-term and long-term benefits according to several embodiments. Beneficial effects from use of the formulations described herein occur upon use, within hours of use, within 1-2 days, 3-4 days, about 7 days, about 14 days, or within about 3 weeks. In several embodiments, the skin characteristics continue to improve over about 2-4 weeks, about 4-6 weeks, about 6-10 weeks, or about 12-16 weeks of use.

In some embodiments, the formulations described herein can be used as leave in formulations on the skin or hair or can be washed off after application. Further, the formulations can be applied on a cleansed skin or a non-cleansed skin. The time between the application and washing off the formulation can be within several minutes of use (e.g., 2, 3, 4, 5, 6, 7, 8, 9, 10, or more minutes), within an hour of use, within 2 hours, 3 hours, about 5 hours. In several embodiments, the formulations provided for herein are suitable for 1, 2, or 3× daily topical application. In one embodiment, the formulation is left on overnight to provide benefits. In several embodiments, the formulations are able to be used on wet or dry skin.

In some embodiments, after the formulation is applied to the skin, the formulation can be removed from the skin with water. In certain embodiments, the formulations can be removed without water, which can be accomplished by massaging the formulation onto the skin, and, for example, wiping any excess formulation with a dry or wet cloth, paper towel or facial tissue. In several embodiments, the formulations are meant to be applied to the skin, or hair, but not purposefully removed (e.g., a‘leave in’ formulation).

According to several embodiments, the formulations described herein can be applied by hand, by sponge, by spraying, by applicator, such as a dropper, by brush, or through use of a composition-impregnated wipe or tissue.

In addition, in some embodiments, at least one of the following ingredients is included: a skin conditioning agent, a solvent, a silicone, an emollient, a preservative, a thickener, an antioxidant, an anti-inflammatory agent, and an excipient. In several embodiments, the formulations are silicone and/or preservative-free. Colorants and fragrances may additionally be included. In some embodiments, the formulation further includes one or more of the following: amino acids, peptides, phospholipids, additional vitamins, growth factors, and additional anti-aging compounds. Surfactants, gelling agents, and pH balancers may also be included.

In some embodiments, the formulations described herein can optionally be applied by transdermal patch. Hydrocolloid or other spot patches are embedded with certain formulations described herein in one embodiment. Pre-treated (e.g., pre-wetted) pads or cloths are provided in some embodiments wherein the pads or cloths are soaked in or embedded with the formulations described herein. In some embodiments, the formulations are absorbent (e.g., readily absorbable) such that no separate means are needed to enhance absorption. However, in alternate embodiments, one or more of massage, mechanical manipulation, energy, or vibration may be used to facilitate absorption. In some embodiments, the formulation is useful post-surgery or dermatological treatment. Further, some of the formulations described herein may be suitable for use for application under one or more skin layers (e.g., as an injectable or micro needle patch).

In some embodiments, the formulation comprises ingredients from Table 1A below:

TABLE 1A

| Example Purpose | | |
|---|---|---|
| In Some | Ingredients In | Example Benefits In |
| Embodiments | Some Embodiments | Some Embodiments |
| Multi-Level | glycerin | moisturizing; |
| Hydration | apple extract | skin softness, antioxidant activity. |
| | water | hydration delivery and retention. |
| | Tremella fuciformis sporocarp extract, | moisture retention, |
| | betaine | stimulate production of hyaluronate |
| | squalane | receptor protein |
| | sodium hyaluronate (for example a 1, 2 | |
| | or 3 molecular weight content. e.g., | |
| | low, medium and high medium weight | |
| | HA) | |
| Barrier | caprylic/capric triglyceride | Balancing epidermal maturation; |
| Defense | cetyl palmitate | improving uniformity of skin; skin |
| | sorbitan stearate | appears more clear and has a more |
| | polysorbate 80 | natural glow; epigenetic, molecular, and |
| | hydrogenated lecithin | cellular regulation of skin renewal; |
| | palmitoyl tetrapeptide-10 | enhanced moisturization, Epidermal |
| | sodium benzoate | refinement and smoothness; stabilize |
| | tocopherol | microbiome; upregulation of laminins or |
| | citric acid | other ECM components; down-regulate |
| | water | proteinases like MMP1; antioxidant, anti- |
| | propanediol | inflammatory, free radical quenching |
| | Lycium barbarum (goji) fruit extract | effects; environment that allows |
| | Lactobacillus ferment | beneficial biodiversity to thrive while |
| | squalane | discouraging harmful bacteria; |
| | | normalizes skin flora, improves barrier |
| | | function. |
| Soothe & | Opuntia ficus-indica stem extract | soothes and calms irritated skin; |
| Protect | Opuntia streptacantha extract (e.g., | hydrates skin; protects and firms skin; |
| | stem extract) | antioxidant; anti redness, anti |
| | Glycerin | photoaging; prepares, protects, and |
| | Phenoxyethanol | repairs the skin from light damage (UV, |
| | Disodium Lauriminodipropionate | IR, and blue light-induced damage); |
| | Tocopheryl Phosphates | reduction in signs of digital and |
| | Phenoxyethanol | photoaging. |
| | Benzoic Acid | |
| | Dehydroacetic Acid | |
| | Water | |
| | Hydrolyzed Pea Protein | |
| | Glucose | |
| | Sodium Chloride | |
| | Sodium Succinate | |

### EXAMPLE

In the table above the “multi-level hydration ingredients” are provided in a range of 5-25% (e.g., 5-15%, 8-10%, 9-12%, 10-20%, 15-20% and overlapping ranges therein). In the table above the “barrier defense ingredients” are provided in a range of 2-20% (e.g., 2-8%, 6-8%, 8-10%, 10-15%, 15-20% and overlapping ranges therein). In the table above the “soothe and protect ingredients” are provided in a range of 3-20% (e.g., 3-8%, 5-10%, 7-10%, 10-15%, 15-20% and overlapping ranges therein). With respect to ingredient(s) that are grouped by function, or other characterization of an ingredient provided for herein, unless otherwise expressly stated, that grouping does not preclude that ingredient from being grouped under, or performing, another function. For example, a solvent may, in several embodiments, also function as an emollient, or a probiotic may also function as a postbiotic, and vice versa.

Optionally, one or more of the following ingredients are also provided in addition to or substitutes for ingredients on Table 1A vitamins such as vitamin D-based compound, vitamin C-based compound, vitamin K-based compound, vitamin B12-based compound (including for example vitamin D, C, K and B12, pro vitamins, analogues, and other derivatives), Armica extract (root, bark, seed or fruit extract), Centella Asiatica extract (root, bark, seed or fruit extract), copper based compound, potassium based compound, ceramides, a multi-mineral complex comprising sodium PCA (pyrrolidone carboxylic acid), zinc based compound such as zinc PCA, magnesium-based compound (such as magnesium), calcium-based compound (such as calcium) and manganese-based compound (such as manganese). In some embodiments, the ceramides comprise or consist essentially of: ceramide NP, ceramide AP, ceramide EOP, phytosphingosine, cholesterol, water, sodium lauroyl lactylate, carbomer and xantham gum. Other ingredients that are optionally included comprise one or more of the following, which may be particularly helpful to reduce or treat acne: alpha-hydroxy acids (AHAs), beta-hydroxy acids (BHAs) including salicylic acid, sulfur, benzoyl peroxide, azelaic acid, niacinamide, retinoids (such as retinal, retinol, adapalene), and tea tree oil. The anti-acne agents are formulated and balanced within the formulation such that the healthy microbiome is still preserved in several embodiments. In several embodiments, these additional ingredients are each provided in a range of about 0.001-10% of the total formulation (e.g., 0.001-0.01%, 0.01-0.05%, 0.05-1%, 0.5-1%, 1-2%, 2-3%, 3-5%, 5-10% and overlapping ranges therein). In one embodiment, an effective amount of an ingredient to treat acne is provided that is at least 1% of the formulation (e.g., 1-10%, 1-15%, or 1-20%).

In several embodiments, the vitamin C based compound comprises at least one of L-ascorbic acid, sodium ascorbyl phosphate, ascorbyl palmitate, magnesium ascorbyl phosphate, ascorbyl methylsilanol pectinate, and tetrahexyldecyl ascorbate. The vitamin C based compound may be used, for example, in a range of about 1-20% of the formulation. In some embodiments, ferulic acid may be added, e.g., in a range of about 0.1-5% of the formulation.

In several embodiments, a mist (or other spray), toner or serum is provided wherein the formulations include the ingredients in Table 1A, except that phenethyl alcohol, gluconolactone, calcium gluconate, polyglycerol-10 oleate, glutamylamidoethyl Imidazole, pentylene glycol, fructooligosaccharides, glycoproteins, bioflavonoids, Cyamopsis tetragonoloba (Guar) gum, xanthan gum, phytic Acid and ethylhexylglycerin are excluded and water, propanediol, Lycium barbarum (Goji) fruit extract, sodium benzoate, Opuntia ficus-indica stem extract, glycerin, hydrolyzed pea protein, glucose, sodium chloride, sodium succinate, sodium hyaluronate, Lactobacillus ferment, squalane, sodium benzoate, glycerin, phenoxyethanol and tocopherol are included.

For example, such formulations can include the following ranges of ingredients: water in the range of 60-95% (e.g., about 65-90%), hydrolyzed pea protein in the range of 0.0001-4% (e.g., about 0.0003-2%), squalane in the range of 0.1-4% (e.g., about 0.5-3%), Lactobacillus ferment in the range of 0.1-4% (e.g., about 0.5-3%), Glycerin in the range of 2-8% (e.g., about 2-7%), Lycium barbarum (Goji) fruit extract in the range of 0.001-2% (e.g., about 0.01-1%), Opuntia extract (such as Opuntia ficus-indica stem extract or prickly pear cactus extract) in the range of 0.001-2% (e.g., 0.001-1%) and one or more ingredients selected from the group consisting or consisting essentially of propanediol, sodium benzoate, glucose, sodium chloride, sodium succinate, sodium hyaluronate, phenoxyethanol, tocopherol, phenethyl alcohol, gluconolactone, calcium gluconate, polyglycerol-10 oleate, glutamylamidoethyl Imidazole, pentylene glycol, fructooligosaccharides, glycoproteins, bioflavonoids, Cyamopsis tetragonoloba (Guar) gum, xanthan gum, phytic Acid and ethylhexyl glycerin in a collective range of about 0.1-10% (e.g., about 2-8%).

In some embodiments the mist (or other spray), toner or serum are configured to stay on the skin to hydrate the skin and balance the microbiome. In other embodiments, a cleansing mist (or other spray) and toner are provided to remove make up and sunscreen. In other embodiments, a formulation, including but not limited to a spray (such as a fine mist or thicker serum), serum, cream, lotion, toner, mask, gel, patch, etc. are provided to stimulate (e.g., store, synthesize, activate, release and/or otherwise enhance) vitamin D and or a precursor of vitamin D synthesis. Such formulations, through the beneficial effects of vitamin D, result in one or more of the following advantages: protection against UV damages through melanin production, (ii) reduction in inflammation, (iii) enhancement of skin barrier (iv) enhancement of immune system, (v) production of new skin cells, and (vi) repair of damaged skin cells. In one embodiment, a combination of ingredients in the formulation including for example fructooligosaccharides, glycoproteins, and/or bioflavonoids work synergistically with other ingredients to stimulate vitamin D.

Topical formulations may be embedded, soaked or otherwise integrated into applicators such as pads (e.g. cotton, cotton blend, bamboo, hemp, synthetic pads) and other applicators, such as patches, micro needle delivery systems, etc. Encapsulated and/or time release formulations are provided in several embodiments.

In some embodiments, the formulation comprises, consists essentially of or consists of several (e.g., 6, 8, 10) or all of the following groups of ingredients listed in Table 1B below.

TABLE 1B

| | | % in total formula in some embodiments |
|---|---|---|
| | | as, for example, % m/m, % m/v, % w/w, |
| Group | Example ingredients in each group | % w/v or % v/v |
| Group | One or more solvents such as water, siloxanes | about 0.001-80% (e.g., about 0.001, 0.005, |
| 1A | (e.g., cyclopentasiloxane), dimethicone and/or | 0.01, 0.02, 0.1, 0.5, 1, 2, 5, 10, 20, 30, 40, |
| | alkane-dimethicone blends, isododecane, | 50, 55, 60, 65, 70, 75, 80%) |
| | caprylic/capric triglycerides, propanediol, pentylene | |
| | glycol, and/or glycerin | |
| Group | a silicone elastomer blend to, for example, act as | about 0.5-20% (e.g., about 0.5, 1, 2, 3, 4, 5, |
| 1B | an emollient, skin conditioning agent, skin | 6, 7, 8, 9, 10, 11, 12, 15%) |
| | softening agent, dispersing agent and/or viscosity | |
| | increasing agent. these include, for example, | |
| | isononyl isononanoate, siloxanes (e.g., | |
| | cyclopentasiloxane), dimethicone crosspolymer, | |
| | dimethicone/vinyl dimethicone crosspolymer, | |
| | and/or dimethiconol, methyl glucose | |
| | caprate/caprylate/oleate | |
| Group | one or more skin conditioning and/or emollient | about 0.001-30% (e.g., about 0.001, 0.005, |
| 1C | agents such as caprylic/capric triglyceride, pyrus | 0.01, 0.1, 0.15, 0.2, 1, 1.5, 2, 2.5, 3, 4, 5, 7, |
| | malus (apple) fruit extract, cetyl palmitate, jojoba | 9, 11, 13, 15, 18, 20, 22, 25, 28, 30%) |
| | esters, glyceryl behenate/eicosadioate, lauryl | |
| | polyglyceryl-3 polydimethylsiloxyethyl dimethicone, | |
| | tocopherol, lauroyl lysine, palmitoyl tripeptide-5, | |
| | glycerin, tribehenin, squalane, disodium | |
| | lauriminodipropionate tocopheryl phosphates, | |
| | panthenol, pantolactone, glycereth-18 | |
| | ethylhexanoate, glycereth-18, gluconolactone, | |
| | calcium gluconate, Opuntia Ficus-Indica stem | |
| | extract, sodium hyaluronate, polyglyceryl-10 | |
| | oleate, glutamylamidoethyl imidazole, pentylene | |
| | glycol, fructooligosaccharides, glycoproteins, | |
| | bioflavonoids, xanthum gum, tocopherol, and/or | |
| | ethylhexylglycerin, | |
| Group | one or more agents for anticaking, bulking and/or | about 0.1-4% (e.g., about 0.5, 0.6, 0.8, 1, |
| 1D | opacifying such as silica, mica and/or alumina | 1.2, 1.5, 2, 2.5, 3%) |
| Group | one or more agents that act as a preservative | about 0.001-4% (e.g., about 0.001, 0.002, |
| 1E | and/or biocide such as phenoxyethanol, sodium | 0.003, 0.004, 0.005, 0.01, 0.02, 0.04, 0.06, |
| | benzoate, pentylene glycol, benzoic acid, citric | 0.08, 0.1, 0.5, 0.8, 1, 1.2, 1.5, 2, 2.5, 3%) |
| | acid, dehydroacetic acid, and/or potassium sorbate | |
| Group | one or more dispersing agents such as silica, | about 0.001-2% (e.g., about 0.001, 0.004, |
| 1F | hydrogenated lecithin, polyhydroxystearic acid | 0.006, 0.008, 0.01, 0.05, 0.1, 0.2, 0.5, 0.6, 1, |
| | | 1.5, 2%) |
| Group | one or more agents for adjusting pH and/or | about 0.001-2% (e.g., about 0.001, 0.004, |
| 1G | chelation such as benzoic acid, gluconolactone, | 0.006, 0.008, 0.01, 0.05, 0.1, 0.2, 0.5, 0.6, 1, |
| | calcium gluconate, phytic acid, sodium hydroxide, | 1.5, 2%) |
| | citric acid, trisodium ethylenediamine disuccinate | |
| Group | one or more agents that act as a humectant such | about 0.0001-20% (e.g., about 0.0001, |
| 1H | as sucrose, glucose, sea water, sorbital, betaine, | 0.0005, 0.001, 0.002, 0.003, 0.005, 0.01, |
| | fructooligosaccharides,phytic acid, Tremella (e.g., | 0.05, 0.1, 0.15, 0.2, 0.5, 1, 2, 5, 6, 7, 8, 9, |
| | Tremella fuciformis sporocarp), glycerin, | 10, 11, 12, 15, 20%) |
| | pantolactone, and/or panthenol | |
| Group | one or more viscosity balancing (increasing or | about 0.001-25% (e.g., about 0.001, 0.002, |
| 1I | decreasing) agents such as sodium chloride, | 0.004, 0.006, 0.008, 0.01, 0.05, 0.1, 0.5, 1, |
| | cetearyl alcohol, sodium polyacryloyldimethyl | 1.5, 2, 3, 5, 6, 7, 8, 9, 10, 11, 12, 15, 20, |
| | taurate, dimethicone (e.g., lauryl polyglyceryl-3 | 25%) |
| | polydimethylsiloxyethyl dimethicone), algin (e.g., | |
| | hydrolyzed algin), hydrogenated polyisobutene, | |
| | propanediol and/or glycerin | |
| Group | one or more agents that act as a binder such as | about 0.001-2% (e.g., about 0.005, 0.01, |
| 1J | triethoxycaprylylsilane, sodium | 0.02, 0.03, 0.05, 0.1, 0.2, 0.5, 1, 1.5, 2, 3%) |
| | polyacryloyldimethyl taurate, Cyamopsis | |
| | Tetragonoloba (Guar) Gum and/or xanthum gum | |
| Group | one or more surfactant and/or solubilizing agents | about 0.001-5% (e.g., about 0.005, 0.01, |
| 1K | such as sorbitan stearate, polyglyceryl-10 oleate, | 0.02, 0.03, 0.05, 0.1, 0.2, 0.5, 1, 1.5, 2, 2.5, |
| | xanthum gum, potassium cetyl phosphate, | 3, 3.5, 4, 5%) |
| | dimethicone (e.g., lauryl polyglyceryl-3 | |
| | polydimethylsiloxyethyl dimethicone), and/or | |
| | glyceryl behenate/eicosadioate | |
| Group | one or more surfactant and/or cleansing agents | about 0.001-5% (e.g., about 0.005, 0.01, |
| 1L | such as sodium lauroyl sarcosinate, | 0.02, 0.03, 0.05, 0.1, 0.2, 0.5, 1, 1.5, 2, 2.5, |
| | cocamidopropyl betaine, | 3, 3.5, 4, 5%) |
| | cocamidopropyl hydroxysultaine, polyglyceryl-10 | |
| | oleate, xanthum gum, and/or | |
| | cocamidopropylamine oxide | |
| Group | one or more agents that act as gelling agent or | about 0.001-5% (e.g., about 0.005, 0.01, |
| 1M | thickener such as sodium polyacryloyldimethyl | 0.02, 0.03, 0.05, 0.1, 0.2, 0.5, 1, 1.5, 2, 2.5, |
| | taurate | 3, 3.5, 4, 5%) |
| Group | one or more agents that act as emulsion | about 0.001-12% (e.g., about 0.005, 0.01, |
| 1N | stabilizing, opacifying, surfactant and/or foam | 0.02, 0.03, 0.05, 0.1, 0.2, 0.5, 1, 1.5, 2, 2.5, |
| | boosting such as cetyaryl alcohol, sodium | 3, 3.5, 4, 5, 6, 6.5, 7, 8, 9, 10, 11, 12%) |
| | polyacryloyldimethyl taurate, glycerin, potassium | |
| | cetyl phosphate, sorbitan stearate, cocamidopropyl | |
| | betaine, sodium lauroyl sarcosinate, Cyamopsis | |
| | Tetragonoloba (Guar) gum, xanthum gum, | |
| | cocamidopropyl hydroxysultaine, | |
| | cocamidopropylamine oxide | |
| Group | one or more fragrance ingredients such as | about 0.0001-5% (e.g., about 0.0001, 0.005, |
| 1O | phenoxyethanol, caprylic/capric triglyceride, | 0.01, 0.02, 0.03, 0.05, 0.1, 0.2, 0.5, 1, 1.5, 2, |
| | benzoic acid, cetyl palmitate, sorbitan stearate, | 2.5, 3, 3.5, 4, 5%) |
| | tocopherol, Cyamopsis Tetragonoloba (Guar) gum, | |
| | sodium benzoate, and/or potassium sorbate | |
| Group | one or more flavoring ingredients such as sodium | about 0.001-5% (e.g., about 0.005, 0.01, |
| 1P | chloride | 0.02, 0.03, 0.05, 0.1, 0.2, 0.5, 1, 1.5, 2, 2.5, |
| | | 3, 3.5, 4, 5%) |
| Group | one or more antioxidants such as Opuntia ficus- | about 0.0001-2% (e.g., about 0.0001, 0.001, |
| 1Q | indica stem extract or other extracts of parts of the | 0.005, 0.01, 0.02, 0.03, 0.05, 0.1, 0.2, 0.5, 1, |
| | fruits of the genus Opuntia (including but not | 1.5, 2%) |
| | limited to prickly pear cactus); disodium | |
| | lauriminoproprionate tocopheryl phosphates and/or | |
| | tocopherol | |

In some embodiments, formulations according to Table 1A/1B have a pH at 25 degrees Celsius of about 5.0-7.7 (e.g., 5.0-5.5, 5.5-6.0, 6.0-6.5, 6.5-7.0, 7.0-7.7).

In the Tables above, the ranges provided in the parentheticals include the overlapping ranges between the numbers. For example, a range of 20-60% (e.g., about 25, 35, 45, 55%) includes, for example, ranges between 25-35%, 25-55%, 45-55%, 35-55%, etc.). In one embodiment, the percentages provided are % m/m or % w/w.

### Moisturizers and Moisturizing Formulations

Despite the large number of moisturizers, there remains a need for a cosmetic formulation that can not only hydrate the skin, but also improve the overall health of the skin, for example by improving the balance of beneficial and harmful microorganisms of the skin microbiome and/or improving the barrier function of the skin (e.g., to further assist in maintaining the hydration of the skin). Both healthy and damaged skin can benefit from several of the formulations described herein. Damaged skin can include skin with dry or dehydrated skin, hyperpigmentation, UV damage, dry skin, oily skin, acne and/or rosacea. The acid-mantle of the skin is created and/or maintained in several embodiments such that, for example, a fine, slightly acidic film on the skin surface is maintained to act as a barrier to undesired bacteria, viruses and other potential contaminants that might penetrate the skin. In some embodiments, sebum is regulated to help facilitate the acid mantle.

In several embodiments, the moisturizer formulations provided for herein serve to replenish, rebalance and repair and/or prepare the skin of a user. In several embodiments, the formulation provides immediate or substantially immediate moisturization and multi-layer hydration. This rapid effect helps strengthen and protect the skin barrier enabling improved moisture retention in the skin. In several embodiments, the formulation promotes skin integrity and quality by supporting good (e.g., beneficial) bacteria to result in a stable balanced microbiome and improved appearance of smooth and supple skin. In several embodiments, calming botanicals soothe skin while advanced antioxidants neutralize free radical damage and improve skin's resilience to environmental aggressors, including blue (HEV) light (as well as UVA/UVB). In several embodiments, formulations described herein increase the skin's own ability to resist damaging light, such as high-energy visible light (e.g., blue light).

In several embodiments, the formulations comprise a combination of three different molecular weight fractions of hyaluronic acid. In several embodiments, this supports skin barrier function and the skin lipid barrier. In several embodiments, this also helps accelerate generation of new keratinocytes, stimulates the expression of the hyaluronate receptor protein (ECM-111) in epidermal keratinocytes.

In several embodiments, extracts of prickly pear are used, which are rich in fatty acids, particularly linoleic acid, and function in part to promote production of ceramides. In several embodiments, extracts of apple are used, which offer a smooth and soft finish free from tack. Moreover, apple saccharide is known for providing long-lasting hydration to the skin maintaining moisture balance over an extended period. In several embodiments, mushroom extracts are used and provide an exceptional ability to hold and retain water in the skin.

In several embodiments, the prebiotic, probiotic and postbiotic work in synergy to promote skin integrity and quality by supporting good bacteria for a stable, balanced microbiome and improved appearance of smooth and supple skin. Thus, in several embodiments, prebiotics such as, for example Lycium barbarum fruit extract and/or Tremella fuciformis sporocarp extract function, in part, to feed and nourish the good bacteria. In several embodiments, probiotics, such as, for example, Lactobacilli or other bacteria, enable a repopulation of good bacteria. In several embodiments, postbiotics, such as, for example, Lactobacillus ferment and/or squalane provide a supportive environment for the repopulated good bacteria to ensure their ongoing health.

In several embodiments, botanicals work to renew the epidermal layer of the skin, lead to more hydrated, smoother, and more nourished skin. In several embodiments, multifunctional vitamin E derivatives provide multilevel emollient support and rescue against various stresses. In several embodiments, certain proteins, such as pea protein, improves skin adaptive responses to increase protection from both indoor and outdoor light damage.

In some embodiments, the formulation comprises, consists essentially of or consists of several (e.g., 8, 10, 12) or all of the following groups of ingredients listed in Table 2 below that, for example, provide moisturization:

TABLE 2

| | | % in total formula in some embodiments as, for |
|---|---|---|
| Group | Example ingredients in each group | example, % m/m, % m/v, % w/w, % w/v or % v/v |
| Group 2.1 | water | about 20-80% (e.g., about 25, 35, 45, 55, 60, 65, 70, |
| | | 75, 80%) |
| Group 2.2 | isononyl isononanoate | about 0.5-5% (e.g., about 0.5, 1, 1.5, 2, 2.5, 3, 3.5, 4, |
| | | 4.5, 5%) |
| Group 2.3 | cetearyl alcohol | about 0.5-5% (e.g., about 0.5, 1, 1.5, 2, 2.5, 3, 3.5, 4, |
| | | 4.5, 5%) |
| Group 2.4 | One or both of: Pyrus malus (Apple) | about 1-7% (e.g., about 1, 2, 3, 4, 5, 6, 7%) as a group, |
| | fruit extract and glycerin | wherein individual ingredients are about 1-5% (e.g., |
| | | about 1,1.5, 2, 2.5, 3, 4, 6, 7%) |
| Group 2.5 | potassium cetyl posphate | about 0.5-5% (e.g., about 0.5, 1, 1.5, 2, 2.5, 3, 3.5, 4, |
| | | 4.5, 5%) |
| Group 2.6 | tribehenin | about 0.5-5% (e.g., about 0.5, 1, 1.5, 2, 2.5, 3, 3.5, 4, |
| | | 4.5, 5%) |
| Group 2.7 | sodium polyacryloyldimethyl taurate | about 0.5-5% (e.g., about 0.5, 1, 1.5, 2, 2.5, 3, 3.5, 4, |
| | | 4.5, 5%) |
| Group 2.8 | squalane | about 0.5-5% (e.g., about 0.5, 1, 1.5, 2, 2.5, 3, 3.5, 4, |
| | | 4.5, 5%) |
| Group 2.9 | at least two (or all of): disodium | about 1-7% (e.g., about 1, 2, 3, 4, 5, 6, 7%) as a group, |
| | lauriminodipropionate tocopheryl | wherein individual ingredients are about 0.001-3% (e.g., |
| | phosphates, phenoxyethanol, water, | about 0.001, 0.002, 0.003, 0.004, 0.005, 0.01, 0.03 0.06, |
| | benzoic acid and dehydroacetic acid | 0.2, 0.5, 0.8, 1, 2, 3%) |
| Group 2.10 | at least two (or all of): caprylic/capric | about 1-7% (e.g., about 1, 2, 3, 4, 5, 6, 7%) as a group, |
| | triglyceride, sorbitan stearate, | wherein individual ingredients are about 0.001-5% (e.g., |
| | polysorbate 80, hydrogenated | about 0.001, 0.002, 0.003, 0.004, 0.005, 0.01, 0.03 0.06, |
| | lecithin, palmitoyl tetrapeptide-10 or | 0.2, 0.5, 0.8, 1, 2, 3, 4, 5%) |
| | other synthetic peptide, sodium | |
| | benzoate, tocopherol, citric acid and | |
| | water | |
| Group 2.11 | at least two (or all of): water | about 1-7% (e.g., about 1, 2, 3, 4, 5, 6, 7%) as a group, |
| | propanediol, Lycium barbarum (Goji) | wherein individual ingredients are about 0.001-3% (e.g., |
| | fruit extract, gluconolactone, sodium | about 0.001, 0.002, 0.003, 0.004, 0.005, 0.01, 0.03 0.06, |
| | benzoate and calcium gluconate | 0.2, 0.5, 0.8, 1, 2, 3%) |
| Group 2.12 | One or both of: Lactobacillus ferment | about 0.1-4% (e.g., about 0.5, 0.9, 1, 1.1, 1.5, 2, 3, 4%) |
| | and phenoxyethanol | as a group, |
| | | wherein individual ingredients are about 0.001-2% (e.g., |
| | | about 0.001, 0.002, 0.005, 0.007, 0.01, 0.02, 0.03, 0.06, |
| | | 0.09, 0.1, 0.2, 0.4, 0.5, 0.7, 0.8, 1, 1.5, 2%) |
| Group 2.13 | At least two (or all of): water, Opuntia | about 0.1-4% (e.g., about 0.1, 0.5, 0.9, 1, 1.1, 1.5, 2, 3, |
| | ficus-indica stem extract or other | 4%) as a group, |
| | extracts of parts of the fruits of the | wherein individual ingredients are about 0.001-2% (e.g., |
| | genus Opuntia (including but not | about 0.001, 0.002, 0.003, 0.004, 0.007, 0.01, 0.02, |
| | limited to prickly pear cactus) Opuntia | 0.03, 0.06, 0.08, 0.09, 0.1, 0.2, 0.5, 0.7, 0.8, 1, 1.5, 2%) |
| | ficus-indica stem extract, glycerin, | |
| | and phenoxyethanol | |
| Group 2.14 | at least two (or all of): water, sodium | about 0.1-4% (e.g., about 0.1, 0.5, 0.9, 1, 1.1, 1.5, 2, 3, |
| | chloride, hydrolyzed pea protein, | 4%) as a group, |
| | glucose, glycerin, and sodium | wherein individual ingredients are about 0.0001-3% |
| | succinate | (e.g., about 0.0001, 0.0002, 0.0005, 0.001, 0.005, 0.01, |
| | | 0.02, 0.03, 0.04, 0.05, 0.06, 0.08, 0.1, 0.4, 0.5, 0.7, 0.8, |
| | | 1, 1.5, 2, 3%) |
| Group 2.15 | at least two (or all of): water, glycerin, | about 0.1-4% (e.g., about 0.1, 0.5, 0.9, 1, 1.1, 1.5, 2, 3, |
| | betaine, Tremella fuciformis | 4%) as a group, |
| | sporocarp extract, glyceryl caprylate | wherein individual ingredients are about 0.0001-3% |
| | and glyceryl undecylenate | (e.g., about 0.0001, 0.0002, 0.0005, 0.001, 0.005, 0.01, |
| | | 0.02, 0.03, 0.04, 0.05, 0.06, 0.08, 0.1, 0.4, 0.5, 0.7, 0.8, |
| | | 1, 1.5, 2, 3%) |
| Group 2.16 | at least one (or all of): water, sodium | about 0.01-4% (e.g., about 0.01, 0.1, 0.2, 0.4, 0.5, 0.9, |
| | hyaluronate (which can be a single or | 1, 1.1, 1.2, 1.5, 2, 3, 4%) as a group, |
| | different molecular weights include a | wherein individual ingredients are about 0.001-2% (e.g., |
| | bi and tri hyaluronate) and | about 0.001, 0.002, 0.009, 0.01, 0.02, 0.08, 0.1, 0.2, |
| | phenoxyethanol | 0.4, 0.7, 0.8, 0.9, 1, 1.5, 2%) |
| Group 2.17 | one or both of glyceryl caprylate and | about 0.1-5% (e.g., about 0.1, 0.2, 0.4, 0.5, 0.9, 1, 1.1, |
| | glyceryl undecylenate | 1.2, 1.5, 2, 3, 4, 5%) as a group, |
| | | wherein individual ingredients are about 0.001-3% (e.g., |
| | | about 0.001, 0.002, 0.009, 0.01, 0.02, 0.08, 0.1, 0.2, |
| | | 0.4, 0.7, 0.8, 0.9, 1, 1.5, 2, 3%) |
| Group 2.18 | silica | about 0.1-5% (e.g., about 0.1, 0.2, 0.5, 1, 1.5, 2, 2.5, 3, |
| | | 3.5, 4, 4.5, 5%) |
| Group 2.19 | one or both of water and trisodium | about 0.1-3% (e.g., about 0.1, 0.2, 0.4, 0.5, 0.9, 1, 1.1, |
| | ethylenediamine disuccinate | 1.2, 1.5, 2, 3%) as a group, |
| | | wherein individual ingredients are about 0.01-3% (e.g., |
| | | about 0.01, 0.02, 0.08, 0.1, 0.2, 0.4, 0.7, 0.8, 0.9, 1, 1.5, |
| | | 2, 3%) |

In some embodiments, formulations according to Table 2 have a pH at 25 degrees Celsius of about 5.0-7.7 (e.g., 5.0-5.5, 5.5-6.0, 6.0-6.5, 6.5-7.0, 7.0-7.7)).

In the table above, the ranges provided in the parentheticals include the overlapping ranges between the numbers. For example, a range of 20-60% (e.g., about 25, 35, 45, 55%) includes, for example, ranges between 25-35%, 25-55%, 45-55%, 35-55%, etc.). In one embodiment, the percentages provided are % m/m or % w/w.

In one embodiment, the formulation is configured for treating skin through use as a moisturizer, for example for hydrating and/or moisturizing the skin, including in some embodiments improvements in the barrier function of the skin (e.g., through balancing and/or improving the skin microbiome).

In some embodiments, a formulation comprising the ingredient groups of Table 2 is adapted for acne-prone skin is provided and further includes one or more of the following ingredients: alpha-hydroxy acids (AHAs), beta-hydroxy acids (BHAs) including salicylic acid, sulfur, benzoyl peroxide, azelaic acid, niacinamide, retinoids (such as retinal, retinol, adapalene), and tea tree oil. In several embodiments, these additional ingredients are each provided in a range of about 0.05-10% of the total formulation (e.g., 0.05-1%, 0.5-1%, 1-2%, 2-3%, 3-5%, 5-10%, and overlapping ranges therein). In one embodiment, an effective amount of an ingredient to treat acne is provided that is at least 1% of the formulation (e.g., 1-10%, 1-15%, or 1-20%).

In one embodiment, the formulation comprises or consists essentially of water, isononyl isononanoate, cetearyl alcohol, glycerin, Pyrus malus (Apple) fruit extract, potassium cetyl phosphate, tribehenin, glyceryl caprylate, Lactobacillus ferment, hydrolyzed pea protein, squalane, disodium lauriminodipropionate tocopheryl phosphates, palmitoyl tetrapeptide-10 and one or more ingredients selected from the group consisting or consisting essentially of sodium polyacryloyldimethyl taurate, phenoxyethanol, benzoic acid, dehydroacetic acid, caprylic/capric triglyceride, cetyl palmitate, sorbitan stearate, polysorbate 80, hydrogenated lecithin, sodium benzoate, tocopherol, citric acid, propanediol, Lycium barbarum (Goji) fruit extract, gluconolactone, calcium gluconate, phenoxyethanol, Opuntia extract (such as Opuntia ficus-indica stem extract or prickly pear cactus extract), glucose, sodium chloride, sodium succinate, Tremella fuciformis sporocarp extract, glyceryl undecylenate, sodium hyaluronate, silica, trisodium ethylenediamine disuccinate.

In several embodiments, formulations for use herein comprises or consists essentially of (i) one or more fruit, cactus or mushroom extracts (such as Pyrus malus (Apple) fruit extract, Lycium Barbarum (goji) fruit extract), Tremella fuciformis sporocarp extract, Opuntia extract (such as Opuntia ficus-indica stem extract or prickly pear cactus extract), and (ii) one or more bacterial products (such as Lactobacillus ferment), and (iii) several (e.g., at least 80%, 85%, 90%, 95%) or all of the following ingredients that work together to help the skin's barrier or otherwise contribute to the benefits as described herein: squalane, hydrolyzed pea protein, and sodium hyaluronate, water, glycerin, cetearyl alcohol, isononyl isononanoate, potassium cetyl phosphate, tribehenin, glyceryl caprylate, sodium polyacryloyldimethyl taurate, disodium lauriminodipropionate tocopheryl phosphates, silica, propanediol, trisodium ethylenediamine disuccinate, caprylic/capric triglyceride, betaine, glyceryl undecylenate, cetyl palmitate, sorbitan stearate, polysorbate 80, calcium gluconate, gluconolactone, citric acid, lecithin (such as hydrogenated lecithin), glucose, tocopherol, sodium chloride, palmitoyl tetrapeptide-10, sodium succinate, phenoxyethanol, sodium benzoate, benzoic acid, and dehydroacetic acid. Such formulations, according to several aspects, include several or all of the following benefits: provide lightweight multi-layer hydration, improve skin's ability to attract and retain hydration, nourish dry, patchy, or flaky skin, prepare skin to respond to exposure to indoor and outdoor light, including UV and HEV (blue) light, and continue to improve resilience and adaptive response over time, prepare skin for optimal sunscreen application, support good bacteria for a stable, balanced microbiome to promote skin integrity and quality, soothe skin, neutralize free radicals to prevent from visible skin damage, layer well over treatment products and under sunscreen and makeup, oil-free, non-comedogenic, non-greasy, fragrance-free, hypoallergenic, and vegan.

Optionally, one or more of the following ingredients are also included: vitamin D based compounds, vitamin C based compounds, vitamin K based compounds, vitamin B12 based compounds, Arnica extract (root, bark, seed or fruit extract), Centella Asiatica extract (root, bark, seed or fruit extract), copper, potassium, ceramides, a multi-mineral complex comprising sodium PCA (pyrrolidone carboxylic acid), zinc PCA, magnesium-based compound (such as magnesium), calcium-based compound (such as calcium) and manganese-based compound (such as manganese). In some embodiments, the ceramides comprise or consist essentially of: ceramide NP, ceramide AP, ceramide EOP, phytosphingosine, cholesterol, water, sodium lauroyl lactylate, carbomer and xantham gum. Other ingredients that are optionally included comprise one or more of the following, which may be particularly helpful to reduce or treat acne: alpha-hydroxy acids (AHAs), beta-hydroxy acids (BHAs) including salicylic acid, sulfur, benzoyl peroxide, azelaic acid, niacinamide, retinoids (such as retinal, retinol, adapalene), and tea tree oil. The anti-acne agents are formulated and balanced within the formulation such that the healthy microbiome is still preserved in several embodiments. In several embodiments, these additional ingredients are each provided in a range of about 0.001-10% of the total formulation (e.g., 0.001-0.01%, 0.01-0.05%, 0.05-1%, 0.5-1%, 1-2%, 2-3%, 3-5%, 5-10% and overlapping ranges therein). In one embodiment, an effective amount of an ingredient to treat acne is provided that is at least 1% of the formulation (e.g., 1-10%, 1-15%, or 1-20%).

In several embodiments, the Vitamin C based compound comprises at least one of I-ascorbic acid, sodium ascorbyl phosphate, ascorbyl palmitate, magnesium ascorbyl phosphate, ascorbyl methylsilanol pectinate, and tetrahexyldecyl ascorbate. In some embodiments, ferulic acid is also included, e.g., in a range of about 0.1-5% of the formulation. The vitamin C based compound may be used, for example, in a range of about 1-20% of the formulation.

In one embodiment, the formulation comprises or consists essentially of a solvent, one or more conditioning agents, one or more occlusive skin conditioning agents, one or more of preservatives and/or biocides, emollients, humectants, viscosity increasing/decreasing agents, surfactants, conditioners, antioxidants, flavoring agents, emulsifiers, fragrances, chelators, absorbents, and pH adjusters. In some embodiments, the preservatives are antimicrobial. In some embodiments, the preservatives are antibacterial. In some embodiments, the preservatives are antifungal.

In one embodiment, the formulation comprises or consists essentially of (i) water in the range of about 60-90% of the total formulation, (ii) Pyrus malus (Apple) fruit extract in the range of about 1-7% of the total formulation, (iii) Lycium barbarum (Goji) fruit extract in the range of about 0.001-3% of the total formulation, (iv) Lactobacillus ferment in the range of about 0.01-3% of the total formulation, (v) Opuntia extract (such as Opuntia ficus-indica stem extract or prickly pear cactus extract) Opuntia ficus-indica stem extract in the range of about 0.001-2% of the total formulation, (vi) Tremella fuciformis sporocarp (Mushroom) extract in the range of about 0.001-2% of the total formulation, (vii) hydrolyzed pea protein in the range of about 0.001-0.3% of the total formulation, (viii) at least four or all of cetearyl alcohol, glycerin, isononyl Isononanoate, tribehenin, cetyl palmitate, collectively in the range of about 9-20% of the total formulation and (ix) one or more preservatives, viscosity increasing/decreasing agents, surfactants, humectants conditioners, emulsifiers, fragrances, and pH adjusters, collectively in a range of about 10-20% of the total formulation.

In one embodiment, the formulation comprises or consists essentially of Water in the range of 60-90% (e.g., about 65-80%), Isononyl isononanoate in the range of 0.1-5% (e.g., about 1-4%), Cetearyl alcohol in the range of 0.1-5% (e.g., about 1-4%), Glycerin in the range of 2-8% (e.g., about 2-7%), Pyrus malus (Apple) fruit extract in the range of 0.1-5% (e.g., about 1-4%), Potassium Cetyl Phosphate in the range of 2-8% (e.g., about 2-7%), Tribehenin in the range of 0.5-4% (e.g., about 1-3%), Glyceryl Caprylate in the range of 0.1-4% (e.g., about 0.5-3%), Lactobacillus ferment in the range of 0.1-4% (e.g., about 0.5-3%), Hydrolyzed Pea Protein in the range of 0.0001-4% (e.g., about 0.0003-2%), Squalane in the range of 0.1-4% (e.g., about 0.5-3%), Disodium Lauriminodipropionate Tocopheryl Phosphates in the range of 0.1-4% (e.g., about 0.5-3%), Palmitoyl Tetrapeptide-10 in the range of 0.0001-2% (e.g., about 0.0003-2%) and one or more ingredients selected from the group consisting or consisting essentially of Sodium Polyacryloyldimethyl Taurate, Phenoxyethanol, Benzoic Acid, Dehydroacetic Acid, Caprylic/Capric Triglyceride, Cetyl Palmitate, Sorbitan Stearate, Polysorbate 80, Hydrogenated Lecithin, Sodium Benzoate, Tocopherol, Citric Acid, Propanediol, Lycium barbarum (Goji) fruit extract, Gluconolactone, Calcium Gluconate, Phenoxyethanol, Opuntia extract (such as Opuntia ficus-indica stem extract or prickly pear cactus extract) stem extract, Glucose, Sodium Chloride, Sodium Succinate, Tremella fuciformis sporocarp extract, Glyceryl Undecylenate, Sodium Hyaluronate, Silica, Trisodium Ethylenediamine Disuccinate in a collective range of about 0.1-10% (e.g., about 2-8%).

In one embodiment, the formulation comprises or consists essentially of water in the range of 60-90% (e.g., about 65-80%), Glycerin in the range of 2-8% (e.g., about 2-7%), Pyrus malus (Apple) fruit extract in the range of 0.1-5% (e.g., about 1-4%), Tremella fuciformis sporocarp (mushroom) extract in the range of 0.001-5% (e.g., about 0.001-2%), betaine in the range of 0.001-5% (e.g., about 0.005-2%), hydrolyzed pea protein in the range of 0.0001-2% (e.g., about 0.0005-1%), Lycium barbarum (Goji) fruit extract in the range of 0.001-2% (e.g., about 0.01-1%), Opuntia extract (such as Opuntia ficus-indica stem extract or prickly pear cactus extract), Lactobacillus ferment in the range of 0.001-3%, Caprylic/Capric Triglyceride in the range of 0.001-2% (e.g., about 0.001-1%), Cetyl Palmitate in the range of 0.001-2% (e.g., about 0.001-1%), Sorbitan Stearate in the range of 0.001-2% (e.g., about 0.001-1%), Polysorbate 80 in the range of 0.001-2% (e.g., about 0.001-1%), Hydrogenated Lecithin in the range of 0.001-2% (e.g., about 0.001-1%), Palmitoyl Tetrapeptide-10 in the range of 0.0001-2% (e.g., about 0.0003-2%) Sodium Benzoate in the range of 0.001-2% (e.g., about 0.001-1%), Tocopherol in the range of 0.0001-2% (e.g., about 0.0005-1%), and Citric Acid in the range of 0.001-2% (e.g., about 0.001-1%).

In one embodiment, the formulation comprises or consists essentially of water in the range of 60-90% (e.g., about 65-80%), Glycerin in the range of 2-8% (e.g., about 2-7%), Pyrus malus (Apple) fruit extract in the range of 0.1-5% (e.g., about 1-4%), Tremella fuciformis sporocarp (mushroom) extract in the range of 0.001-5% (e.g., about 0.001-2%), betaine in the range of 0.001-5% (e.g., about 0.005-2%), hydrolyzed pea protein in the range of 0.0001-2% (e.g., about 0.0005-1%), Lycium barbarum (Goji) fruit extract in the range of 0.001-2% (e.g., about 0.01-1%), Opuntia extract (such as Opuntia ficus-indica stem extract or prickly pear cactus extract) in the range of 0.001-2% (e.g., about 0.001-1%), and Lactobacillus ferment in the range of 0.001-3% (e.g., about 0.01-3%).

Some embodiments further comprise Isononyl Isononanoate in the range of 0.1-5% (e.g., about 1-4%) and Cetearyl Alcohol in the range of 0.1-5% (e.g., about 1-4%).

Some embodiments further comprise Squalane in the range of 0.1-4% (e.g., about 0.5-3%) and tribehenin in the range of 0.5-4% (e.g., about 1-3%).

In several embodiments, the formulation comprises a humectant. Glycerin can be used herein as a humectant, in several embodiments. In several other embodiments, betaine and/or glucose can be used as a humectant. In several other embodiments, urea, hyaluronic acid, propylene glycol, panthenol, honey and/or sorbitol can be used as a humectant. Some embodiments include salicylic acid, alpha hydroxy acids (AHAs), such as glycolic acid and lactic acid.

In several embodiments, the formulation comprises an occlusive and a skin-protecting agent. Tribehenin, squalane, caprylic/capric triglyceride, cetyl palmitate, tocopherol can be used herein as an occlusive and a skin-protecting agent. In several other embodiments, triheptanoin, tricaprin, camellia seed oil, petroleum jelly, mineral oil, castor oil, argan oil, shea butter, cocoa butter, beeswax or lanolin can be used as an occlusive and a skin-protecting agent.

In several embodiments, the formulation comprises an emollient. Isononyl Isononanoate can be used herein as a humectant. In several other embodiments, propylene glycol, castor oil, dimethicone, paraffin, argan oil, jojoba oil, ceramides or stearic acid can be used as an emollient.

In several embodiments, the water used in the formulation is one or more of distilled, deionized, and/or sea water.

In those embodiments in which Goji extract is provided, the extract may be used, for example, as an antioxidant, to increase photoprotection of the skin and impart anti-aging properties. In some embodiments, Goji extract is obtained from Lycium barbarum L. In several embodiments, the extract is extracted from the cell wall, leaf, root, bark, fruit, flower or other part of the organism. In some embodiments, Goji extract is made with vegetable-derived propanediol. In several embodiments, Goji extract also possesses anti-inflammatory properties.

In those embodiments in which apple fruit extract is provided, the extract may be used, for example, as antioxidants, to increase photoprotection of the skin and impart anti-aging properties. In some embodiments, apple fruit extract is obtained from Pyrus malus. An extract may be obtained, for example, by the technique of extraction or by in vitro plant cell culture.

In several embodiments, one or more prebiotics are included in the formulation. Prebiotics include but are not limited to, mushroom extracts (such as Tremella extracts, e.g., Tremella fuciformis or Tremella fuciformis sporocarp extracts), apple extracts (such as saccharides, Pyrus extracts, Pyrus malus extracts), saccharides including from other fruits such as kiwi, pomegranate, papaya, mango, and/or pear, pectins, mucilage, cactus and other plant extracts, bacterial polysaccharides, plant-based sugar molecules, konjac root extract (e.g., glucomannan), inulin, alpha-glucan, oligosaccharides, polysaccharides, oat extract, black currant extract, Lycium extracts (such as Lycium barbarum (Goji) fruit extracts, as well as other nutrients that feed and support healthy microbial growth.

In several embodiments, the ingredients to soothe and protect comprise at least one vitamin D based compound such as cholecalciferol, calcitriol, calcipotriene 7-Dehydrocholesterol (7-DHC), vitamin D2 or D3 and its analogues, pro vitamin D, etc.). In one embodiment, the vitamin D based compound is provided in the range of 0.01%-10% of the formulation or 250-10,000 IU (e.g., 250-2500, 2500-7500, 7500-10,000 IU) per application. The vitamin D based compound comprises lumisterol in one embodiment.

In several embodiments, one or more probiotics are used alone or in combination with prebiotics and/or postbiotics. Probiotics include but are not limited to L. acidophilus, L. casei, L. casei Shirota, L. fermentum, L. gasseri, L. helveticus, L. johnsonii, L. paracasei, L. plantarum, L. reuteri, L. rhamnosus, L. rhamnosus GG, Bifidobacterium, (e.g., B. sp., B. adolescentis, B. angulatum, B. animalis, B. bifidum, etc.). In some embodiments, these microbes are living microbes. In some embodiments, inactive microbes or portions of these microbes are used as the probiotic. In some embodiments, the metabolic byproducts of these microbes (e.g., components of the cell wall and/or other cellular structures) are used as the probiotics, such as fermentation products (e.g., ferment, ferment lysate, lysate, isolate, etc.). In some embodiments, one or more of teichoic acids such as ribitol teichoic acids, glycerol teichoic acids, lipoteichoic acid, and peptidoglycan(s) are used as probiotics. Gram-positive bacteria, including gram-positive, lactic acid producing bacteria are used in several embodiments. In some embodiments, one, two, three or more species of the same genus are used. In other embodiments, combinations of two or more genera are used (e.g., Lactobacillus and Bifidobacterium for example). In yet other embodiments, combinations of two or more different microbes are used (e.g., bacteria and yeast, for example). In some embodiments, L. plantarum (or its ferment) is not used.

In several embodiments, one or more postbiotics are used alone or in combination with prebiotics and/or probiotics. In some embodiments, ferments of yeast (such as Saccharomyces cerevisiae, S. carlsbergensis, and/or Dekkera/Brettanomyces) are used including but not limited to yeast ferments of sugarcane, such as squalane. Ferments and other byproducts of the beneficial microbes described here are used as postbiotics in some embodiments, wherein such ferments and other byproducts that provide an environment that allows beneficial biodiversity to thrive. In one embodiment, Lactobacillus ferment is used as both a probiotic and postbiotic and squalane is used as a postbiotic. Squalane or other compounds produced through the fermentation of sugarcane, sugar or another saccharide (e.g., mono- or di-saccharide) through yeast (such as Saccharomyces cerevisiae) or by another microbe is used in some embodiments. In one embodiment, ferment functions as an antioxidant activated by heat and light, to protect against UV damage, to preserve and reinforce the skin's natural defenses, and/or to improve epidermal structural integrity.

In some embodiments, rice ferment or sake ferment can be used as a postbiotic. In some embodiments, a yeast derived ferment (e.g., Saccharomyces ferment) can be used as a postbiotic.

In several embodiments, squalane provides several benefits to assist in the support and/or improvement of the skin microbiome, such as i) contaminant-free, ii) increases bacterial diversity without harming growth iii) maintains a healthy microbiome balance and/or variety of bacterial composition, and iv) does not disturb skin balance. Squalane provides postbiotic activity in some embodiments. Squalene is provided in alternative embodiments.

In several embodiments, the formulation comprises one or more vitamin E-based compounds. In several embodiments, the vitamin E compound comprises tocopherol, tocopheryl, tocopheryl phosphate or other derivative thereof. In several embodiments, the vitamin E compound comprises disodium lauriminodipropionate tocopheryl phosphate.

In some embodiments, triglycerides are included in the formulation. In one embodiment, triglycerides may be derived from coconut oil or other oils. In one embodiment, the triglycerides comprise caprylic and capric fatty acids.

In one embodiment, the water used in the formulation is one or more of distilled, deionized, and/or sea water.

In one embodiment, a thickener or viscosity increasing agent is provided in a range of about 0.1-15% of the total formulation (e.g., 0.1%, 0.5%, 1%, 2%, 3%, 4%, 5%, 10%, 12%, 15%, and ranges in between). In one embodiment, the percentages provided are % m/m or % w/w.

In several embodiments, the formulation comprises a stabilizer, suspending agent and/or thickener. Sodium polyacryloyldimethyl taurate can be used herein as a stabilizing or a suspending agent or a thickener. In some embodiments, Dimethicone crosspolymer can be used as a stabilizer, suspending agent and/or thickener. Types of dimethicone crosspolymers that can be used as a stabilizing agent, suspending agent or a thickener include, but are not limited to, silicone, dimethicone crosspolymer, cyclopentasiloxane, dimethicone and polysilicone-11 blend, coconut (or other plant or synthetic source) alkane-dimethicone blend, dimethicone crosspolymer 3, dimethicone crosspolymer PEG-8, cetyl dimethicone/dimethicone cross polymer, and dimethicone/vinyl dimethicone crosspolymer, and combinations thereof. In several embodiments, cyclomethicone, cyclotetrasiloxane and/or cyclopentasiloxane are not used.

In several embodiments, a dispersing agent is used to achieve a more even distribution (and stabilization, in some embodiments) of solid particles, like pigments and fillers, throughout the formulation. In several embodiments, dimethicone, silica and polyhydroxystearic acid are used as dispersing agents. In another embodiment, disteardimonium hectorite may be used.

To Applicant's knowledge, several combinations of ingredients disclosed herein represent unique enhanced formulations that are not naturally occurring (e.g., not found in nature in such combination). Moreover, in several embodiments, the individual ingredients may be modified to be structurally and/or functionally different than the naturally-occurring species, thereby resulting in markedly unique effects. Salts and acids of the ingredients identified herein, as applicable, can be used in some embodiments.

In several embodiments, the invention comprises a method of treating skin using any one of the formulations described above or below. The use of any of the formulations described herein for treating skin (e.g., improving skin hydration, balancing the skin microbiome, reducing infection and/or inflammation reducing the appearance of fine lines, laxity, wrinkles, puffiness) and/or improving skin appearance is provided in several embodiments. Several embodiments also include instructing the method or use of the formulation (e.g., via instructions for use).

### Cleansers and Cleansing Formulations

Despite the large number of cosmetic formulations on the market for cleansing, there remains a need for a cosmetic formulation that can cleanse, treat, improve and/or maintain the health of the skin, including maintenance of skin microbiome and/or has antiaging properties. Both healthy and damaged skin can benefit from several of the formulations described herein. Damaged skin can include skin with hyperpigmentation, UV damage, dry skin, oily skin, acne and/or rosacea.

In some embodiments, the formulation comprises, consists essentially of or consists of several (e.g., 6, 8, 10) or all of the following groups of ingredients listed in Table 3 below that, for example, help cleanse the skin.

TABLE 3

| | | % in total formula in some embodiments as, for |
|---|---|---|
| Group | Example ingredients in each group | example, % m/m, % m/v, % w/w, % w/v or % v/v |
| Group 3.1 | water | about 10-40% (e.g., about 10, 15, 20, 25, 35, 40%) |
| Group 3.2 | at least two (or all of): water, | about 5-30% (e.g., about 5, 6, 7, 8, 9, 10, 12, 15, 20, 25, |
| | cocamidopropyl betaine and sodium | 30%) as a group, |
| | chloride | wherein individual ingredients are about 0.05-20% (e.g., |
| | | about 0.05, 0.1, 0.2, 0.4, 0.7, 0.8, 0.9, 1, 5, 10, 15, 20%) |
| Group 3.3 | one or both of water and sodium | about 5-30% (e.g., about 5, 6, 7, 8, 9, 10, 12, 15, 20, 25, |
| | lauroyl sarcosinate | 30%) as a group, |
| | | wherein individual ingredients are about 5-20% (e.g., 5, |
| | | 10, 15, 20%) |
| Group 3.4 | at least two (or all of): water, | about 5-25% (e.g., about 5, 6, 7, 8, 9, 10, 12, 15, 20, |
| | cocamidopropyl hydroxysultaine, | 25%) as a group, |
| | glycerin and sodium chloride | wherein individual ingredients are about 0.1-15% (e.g., |
| | | about 0.1, 0.2, 0.25, 0.8, 1, 3, 4, 6, 7, 8, 12, 14, 15%) |
| Group 3.5 | one or both of: water and | about 1-10% (e.g., about 1, 2, 3, 4, 5, 6, 7, 8, 9, 10%) as |
| | cocamidopropylamine oxide | a group, |
| | | wherein individual ingredients are about 0.1-7% (e.g., |
| | | about 0.1, 0.15, 0.2, 0.25, 0.8, 1, 3, 3.5, 4, 6, 7%) |
| Group 3.6 | one or both of: methyl glucose | about 1-10% (e.g., about 1, 2, 3, 4, 5, 6, 7, 8, 9, 10%) as |
| | caprate/caprylate/oleate and | a group, |
| | propanediol | wherein individual ingredients are about 0.1-7% (e.g., |
| | | about 0.1, 0.15, 0.2, 0.25, 0.8, 1, 3, 3.5, 4, 6, 7%) |
| Group 3.7 | glycerin | about 0.5-5% (e.g., about 0.5, 1, 1.5, 2, 2.5, 3, 3.5, 4, 4.5, |
| | | 5%) |
| Group 3.8 | at least two (or all of): glycerin | about 1-10% (e.g., about 1, 2, 3, 4, 5, 6, 7, 8, 9, 10%) as |
| | water, | a group, |
| | Citrus paradisi (grapefruit) fruit extract | wherein individual ingredients are about 0.001-5% (e.g., |
| | and | about 0.001, 0.005, 0.01, 0.05, 0.1, 0.15, 0.2, 0.25, 0.8, |
| | citric acid | 1, 3, 3.5, 4, 5%) |
| Group 3.9 | one or both of: glycereth-18 | about 1-10% (e.g., about 1, 2, 3, 4, 5, 6, 7, 8, 9, 10%) as |
| | and glycereth-18 ethylhexanoate | a group, |
| | | wherein individual ingredients are about 0.1-7% (e.g., |
| | | about 0.1, 0.15, 0.2, 0.25, 0.8, 1, 3, 3.5, 4, 6, 7%) |
| Group 3.10 | one or both of: Lactobacillus ferment | about 0.01-4% (e.g., about 0.01, 0.1, 0.2, 0.4, 0.5, 0.9, 1, |
| | and phenoxyethanol | 1.1, 1.2, 1.5, 2, 3, 4%) as a group, |
| | | wherein individual ingredients are about 0.001-2% (e.g., |
| | | about 0.001, 0.002, 0.009, 0.01, 0.02, 0.08, 0.1, 0.2, 0.4, |
| | | 0.7, 0.8, 0.9, 1, 1.5, 2%) |
| Group 3.11 | At least two (or all of): water, Opuntia | about 0.1-4% (e.g., about 0.1, 0.5, 0.9, 1, 1.1, 1.5, 2, 3, |
| | ficus-indica stem extract or other | 4%) as a group, |
| | extracts of parts of the fruits of the | wherein individual ingredients are about 0.001-2% (e.g., |
| | genus Opuntia (including but not | about 0.001, 0.002, 0.003, 0.004, 0.007, 0.01, 0.02, |
| | limited to prickly pear cactus) Opuntia | 0.03, 0.06, 0.08, 0.09, 0.1, 0.2, 0.5, 0.7, 0.8, 1, 1.5, 2%) |
| | ficus-indica stem extract, glycerin, and | |
| | phenoxyethanol | |
| Group 3.12 | one or both of water and trisodium | about 0.1-3% (e.g., about 0.1, 0.2, 0.4, 0.5, 0.9, 1, 1.1, |
| | ethylenediamine disuccinate | 1.2, 1.5, 2, 3%) as a group, |
| | | wherein individual ingredients are about 0.01-3% (e.g., |
| | | about 0.01, 0.02, 0.08, 0.1, 0.2, 0.4, 0.7, 0.8, 0.9, 1, 1.5, |
| | | 2, 3%) |
| Group 3.13 | at least two (or all of): water, | about 0.01-4% (e.g., about 0.01, 0.1, 0.2, 0.4, 0.5, 0.9, 1, |
| | potassium sorbate and sodium | 1.1, 1.2, 1.5, 2, 3, 4%) as a group, |
| | benzoate | wherein individual ingredients are about 0.001-2% (e.g., |
| | | about 0.001, 0.002, 0.009, 0.01, 0.02, 0.08, 0.1, 0.2, 0.4, |
| | | 0.7, 0.8, 0.9, 1, 1.5, 2%) |
| Group 3.14 | at least two (or all of): sorbeth-230 | about 0.1-3% (e.g., about 0.1, 0.2, 0.4, 0.5, 0.9, 1, 1.1, |
| | tetraoleate, decyl glucoside, water and | 1.2, 1.5, 2, 3%) as a group, |
| | sorbitan laurate | wherein individual ingredients are about 0.01-3% (e.g., |
| | | about 0.01, 0.02, 0.08, 0.1, 0.2, 0.4, 0.7, 0.8, 0.9, 1, 1.5, |
| | | 2, 3%) |
| Group 3.15 | citric acid | about 0.01-2% (e.g., about 0.01, 0.02, 0.05, 0.1, 0.2, 0.5, |
| | | 1, 1.5, 2%) |

In some embodiments, formulations according to Table 3 have a pH at 25 degrees Celsius of about 5.0-7.7 (e.g., 5.0-5.5, 5.5-6.0, 6.0-6.5, 6.5-7.0, 7.0-7.7)).

In the table above, the ranges provided in the parentheticals include the overlapping ranges between the numbers. For example, a range of 20-60% (e.g., about 25, 35, 45, 55%) includes, for example, ranges between 25-35%, 25-55%, 45-55%, 35-55%, etc.). In one embodiment, the percentages provided are % m/m or % w/w.

In one embodiment, a cleansing formulation, whether in a gel, foam, leave-on mask, or other topical form, is adapted for acne-prone skin and optionally includes one or more of the following additional ingredients: alpha-hydroxy acids (AHAs), beta-hydroxy acids (BHAs) including salicylic acid, sulfur, benzoyl peroxide, azelaic acid, niacinamide, retinoids (such as retinal, retinol, adapalene), and tea tree oil. In several embodiments, these additional ingredients are each provided in a range of about 0.05-10% of the total formulation (e.g., 0.05-1%, 0.5-1%, 1-2%, 2-3%, 3-5%, 5-10% and overlapping ranges therein). In one embodiment, an effective amount of an ingredient to treat acne is provided that is at least 1% of the formulation (e.g., 1-10%, 1-15%, or 1-20%).

An enhanced glow or radiance is achieved by several embodiments. Many formulations are not only therapeutic and protective, but also nourish the skin, increase hydration, and improve overall appearance, texture and firmness. Thus, several formulations of the invention are beneficial for skin that is aged, sun-damaged, wrinkled, lax, and/or blemished. Formulations according to several embodiments can be used anywhere on the body, and are especially beneficial for the face, neck, décolletage and hands, where signs of skin damage and aging may be particularly prominent.

In one embodiment, the formulation is configured for treatment as a cleansing wash for the body. In other embodiments, the formulation can be used as a face cleanser. In several embodiments, the formulation can be used to remove makeup and/or sunscreen. In several embodiments, the formulation provides hydrating and/or moisturizing effects. In several embodiments, the formulation provides skin firmness. In several embodiments, the formulation has antiaging benefits. In several embodiments, the formulation promotes and/or maintains a balanced skin microbiome to support a healthy skin barrier. In several embodiments, the formulation provides hydrating and/or moisturizing effects without feeling heavy on the skin. In several embodiments, the formulation works at multiple levels of the skin to hydrate, smooth and soften rough texture, and plump appearance of fine lines.

In some embodiments, the invention comprises a topical formulation for cleansing and/or treating skin, such as cleaning, hydrating and/or moisturizing dry, flaky, and/or itchy skin in a subject, wherein the topical formulation is a liquid (such as serum, lotion, liquid, oil or cream), a gel, a spray, a powder, a jelly, or a combination thereof. Physical and/or chemical exfoliants are provided in some embodiments. AHAs, BHAs, or both are provided in some formulations, including cleansing and moisturizing formulations. Pads soaked with the formulations described herein are provided in some embodiments, while in other embodiments patches that can remain on the skin for several hours or overnight that are pre-treated with the formulations described herein are provided. Such patches include, but are not limited to, absorbent patches, hydrocolloidal patches, silicone patches, and hydrogel patches. Some patches are designed to penetrate below the skin surface for effective delivery of ingredients.

In one embodiment, the formulation is configured for treating skin through use as a cleansing wash, for example a hydrating and/or a moisturizing cleanser for the face or body.

In addition, in some embodiments, at least one of the following ingredients is included: a skin conditioning agent, a solvent, a silicone, an emollient, a preservative, a thickener, an antioxidant, an anti-inflammatory agent, and an excipient. In several embodiments, the formulations are silicone and/or preservative-free. Colorants and fragrances may additionally be included. In some embodiments, the formulation further includes one or more of the following: amino acids, peptides, phospholipids, additional vitamins, growth factors, and additional anti-aging compounds. Surfactants, gelling agents, and pH balancers may also be included.

In one embodiment, formulations for cleansing skin (including on the scalp, face and body), or otherwise removing impurities from the skin, comprise or consist essentially of: sodium lauroyl sarcosinate, cocamidopropyl hydroxysultaine, a bacterial product (such as Lactobacillus ferment), and fruit and/or cactus extract (such as grapefruit and prickly pear extract). For example, in one aspect, a cleansing formulation comprises: water, sodium lauroyl sarcosinate, cocamidopropyl betaine, cocamidopropyl hydroxysultaine, glycerin, methyl glucose caprate/caprylate/oleate, sodium chloride, cocamidopropylamine oxide, glycereth-18 ethylhexanoate, propanediol, glycereth-18, Lactobacillus ferment, sorbeth-230 tetraoleate, decyl glucoside, trisodium ethylenediamine disuccinate, citric acid, sorbitan laurate, Citrus paradisi (grapefruit) fruit extract, Opuntia ficus-indica stem extract, sodium benzoate, potassium sorbate and phenoxyethanol. In some aspects, such formulations include several benefits such as: lightweight with a smooth texture that lightly lathers and rinses clean, gently and effectively removes mineral sunscreen, makeup, excess oil, and impurities without leaving skin feeling tight or stripped, can replace a double-cleansing routine, prepare skin for application of skin care treatment products, can support skin's microbiome balance, sooth and protect skin, maintain a natural pH level, oil-free, non-comedogenic, fragrance-free and hypoallergenic.

Optionally, one or more of the following ingredients are also included: vitamin D based compound, vitamin C based compound, vitamin K based compound, vitamin B12 based compound, Arnica extract (root, bark, seed or fruit extract), Centella Asiatica extract (root, bark, seed or fruit extract), copper, potassium, ceramides, a multi-mineral complex comprising sodium PCA (pyrrolidone carboxylic acid), zinc based compound such as zinc PCA, magnesium based compound, calcium based compound and manganese based compound. In some embodiments, the ceramides comprise or consist essentially of: ceramide NP, ceramide AP, ceramide EOP, phytosphingosine, cholesterol, water, sodium lauroyl lactylate, carbomer and xantham gum. Other ingredients that are optionally included comprise one or more of the following, which may be particularly helpful to reduce or treat acne: alpha-hydroxy acids (AHAs), beta-hydroxy acids (BHAs) including salicylic acid, sulfur, benzoyl peroxide, azelaic acid, niacinamide, retinoids (such as retinal, retinol, adapalene), and tea tree oil. The anti-acne agents are formulated and balanced within the formulation such that the healthy microbiome is still preserved in several embodiments. In several embodiments, these additional ingredients are each provided in a range of about 0.001-10% of the total formulation (e.g., 0.001-0.01%, 0.01-0.05%, 0.05-1%, 0.5-1%, 1-2%, 2-3%, 3-5%, 5-10% and overlapping ranges therein). In one embodiment, an effective amount of an ingredient to treat acne is provided that is at least 1% of the formulation (e.g., 1-10%, 1-15%, or 1-20%).

In one embodiment, the formulation comprises or consists essentially of Water, Sodium Lauroyl Sarcosinate, Citrus paradisi (Grapefruit) fruit extract, Opuntia extract (such as Opuntia ficus-indica stem extract or prickly pear cactus extract) t, Glycerin, Lactobacillus ferment, Methyl Glucose Caprate/Caprylate/Oleate and one or more ingredients selected from the group consisting of Cocamidopropyl Betaine, Cocamidopropyl Hydroxysultaine, propanediol, Sodium Chloride, Cocamidopropylamine Oxide, Glycereth-18 Ethylhexanoate, Glycereth-18, Sorbeth-230 Tetraoleate, Sodium Benzoate, Potassium Sorbate, Trisodium Ethylenediamine Disuccinate, Decyl Glucoside, Citric Acid, Sorbitan Laurate, Phenoxyethanol.

In one embodiment, the formulation comprises or consists essentially of a solvent, one or more conditioning agents, one or more of preservatives and/or biocides, emollients, humectants, viscosity increasing/decreasing agents, surfactants, conditioners, antioxidants, flavoring agents, emulsifiers, fragrances, chelators, absorbents, and pH adjusters. In some embodiments, the preservatives is antimicrobial. In some embodiments, the preservatives is antibacterial. In some embodiments, the preservatives is antifungal.

In one embodiment, the formulation comprises or consists essentially of (i) water in the range of about 40-80% of the total formulation, (ii) Sodium Lauroyl Sarcosinate in the range of about 4-12% of the total formulation, (iii) Citrus paradisi (Grapefruit) fruit extract in the range of about 0.001-2% of the total formulation, (iv) Opuntia extract (such as Opuntia ficus-indica stem extract or prickly pear cactus extract) Opuntia ficus-indica stem extract in the range of about 0.001-2% of the total formulation, (v) Glycerin in the range of about 1-8% of the total formulation, (vi) Lactobacillus ferment in the range of about 0.01-5% of the total formulation, (vii) at least four or all of Methyl Glucose Caprate/Caprylate/Oleate, Cocamidopropyl Hydroxysultaine, Cocamidopropylamine Oxide, Glycereth-18 Ethylhexanoate, Glycereth-18, Sorbeth-230 Tetraoleate, Decyl Glucoside, Sorbitan Laurate, collectively in the range of about 10-20% of the total formulation and (viii) one or more preservatives, viscosity increasing/decreasing agents, surfactants, foaming agents, humectants conditioners, emulsifiers, fragrances, and pH adjusters, collectively in a range of about 15-30% of the total formulation.

In one embodiment, the formulation comprises or consists essentially of (i) water in the range of about 40-80% (e.g., about 50-70%) of the total formulation, (ii) Sodium Lauroyl Sarcosinate in the range of about 4-12% (e.g., about 6-11%) of the total formulation, (iii) Citrus paradisi (Grapefruit) fruit extract in the range of about 0.001-2% (e.g., about 0.005-1.5%), of the total formulation, (iv) Opuntia extract (such as Opuntia ficus-indica stem extract or prickly pear cactus extract) in the range of about 0.001-2% (e.g., about 0.005-1.5%) of the total formulation, (v) Glycerin in the range of about 1-8% (e.g., about 2-7%) of the total formulation, (vi) Lactobacillus ferment in the range of about 0.01-5% (e.g., about 0.5-3%) of the total formulation, (vii) at least four or all of Methyl Glucose Caprate/Caprylate/Oleate, Cocamidopropyl Hydroxysultaine, Cocamidopropylamine Oxide, Glycereth-18 Ethylhexanoate, Glycereth-18, Sorbeth-230 Tetraoleate, Decyl Glucoside, Sorbitan Laurate, collectively in the range of about 10-20% of the total formulation and (viii) one or more preservatives, viscosity increasing/decreasing agents, surfactants, foaming agents, humectants conditioners, emulsifiers, fragrances, and pH adjusters, collectively in a range of about 15-30% (e.g., about 10-25%) of the total formulation.

Several embodiments are particularly adapted to calm skin and comprise more than 50%, 75%, 85%, 90%, or 95% of the ingredients provided in the tables provided herein and additionally one or more of the following ingredients: niacinamide, titanium dioxide, zinc oxide, water, glycerin, C13-15 alkane, dimethyl isosorbide, triethylhexanion, caprylic/capric triglyceride, sorbitan stearate, polyglyceryl-2 diisostearate, disodium lauriminodipropionate tocopheryl phosphate, polyacrylate crosspolymer-6, bisabolol, sorbityl laurate, cetyl palmitate, Tremella fuciformis sporocarp (mushroom) extract, magnesium carboxymethyl beta-glucan, polysorbate 80, betaine, citric acid, crithmum maritimum extract, t-butyl alcohol, hydrogenated lecithin, magnolia officinalis bark extract, tocopherol, Zingiber officinale (ginger) root extract, glycolic acid, palmitoyl tetrapeptide-10, sodium benzoate, potassium sorbate, phenoxyethanol, benzoic acid, dehydroacetic acid and chloroacetic acid. In some embodiments, the formulation further comprises a marine plant extract from sea fennel. Such calming formulations, according to several aspects, include several or all of the following benefits: calms and soothes sensitive and irritated skin, skin is cooled upon application, provides a smooth texture where the skin's surface is balanced, uniform and luminous, restores the skin where the skin is deeply hydrated and barrier health is improved to maintain moisturization, diminishes the appearance of redness with continuous use, non-comedogenic, and hypoallergenic.

Several embodiments are particularly adapted to diminish hyperpigmentation and comprise more than 50%, 75%, 85%, 90%, or 95% of the ingredients provided in the tables provided herein and additionally one or more of the following ingredients: titanium dioxide, zinc oxide, cyclopentasiloxane, isocetyl stearoyl stearate, dimethicone crosspolymer, Thermus thermophillus ferment, water, hydrated silica, dimethicone/vinyl dimethicone crosspolymer, disodium lauriminodipropionate tocopheryl phosphates, panthenyl triacetate, rheum extract (such Rheum rhaponticum root extract), Bidens pilosa extract, Elaeis guineensis (palm) oil, Gossypium herbaceum (cotton) seed oil, Linum usitatissimum (linseed) seed oil, tocopherol, dimethiconol, Citrus paradisi (grapefruit) seed extract, glycerin, dimethicone, fusanus spicatus wood oil, Vanilla planifolia fruit extract, ascorbic acid, caprylic/capric triglyceride, pentylene glycol, triethoxycaprylylsilane, acrylates/c12-22 alkyl methacrylate copolymer, phenoxyethanol, benzoic acid, dehydroacetic acid, potassium sorbate, farnesol and iron oxides. Such pigment correcting formulations, according to several aspects, include several or all of the following benefits: provides a broad spectrum sun protection, reduce skin discoloration, water resistant, non-comedogenic, paraben-free and gluten free.

Some embodiments, further comprises Sodium Lauroyl Sarcosinate in the range of about 4-12% (e.g., about 6-11%) of the total formulation and Citrus paradisi (Grapefruit) fruit extract in the range of about 0.001-2% (e.g., about 0.005-1.5%), of the total formulation,

Some embodiments, further comprise Glycereth-18 Ethylhexanoate in the range of 0.1-4% (e.g., about 0.5-3%).

In several embodiments, the water used in the formulation is one or more of distilled, deionized, and/or sea water.

In several embodiments, the formulation comprises a humectant. Glycerin can be used as a humectant for several embodiments. In several other embodiments, betaine and/or glucose can be used as a humectant. In several other embodiments, urea, hyaluronic acid, salicylic acid, alpha hydroxy acids (AHAs), such as glycolic acid and lactic acid, propylene glycol, Panthenol, honey or sorbitol can be used as a humectant.

In several embodiments, the formulation comprises a skin-conditioning agent. Cocamidopropyl Hydroxysultaine, Cocamidopropyl Betaine, glycerin, Glycereth-18 Ethylhexanoate, Opuntia extract (such as Opuntia ficus-indica stem extract or prickly pear cactus extract) Opuntia ficus-indica stem extract can be used herein as a skin-conditioning agent. In several other embodiments, triheptanoin, tricaprin, camellia seed oil, cetyl alcohol, kokum, almond oil, petroleum jelly, mineral oil, castor oil, argan oil, shea butter, cocoa butter, beeswax or lanolin can be used as a skin-conditioning agent.

In several embodiments, the formulation comprises an emollient. Methyl Glucose Caprate/Caprylate/Oleate can be used herein as a humectant. In several other embodiments, Isononyl Isononanoate, propylene glycol, castor oil, dimethicone, paraffin, argan oil, jojoba oil, ceramides or stearic acid can be used as an emollient.

In those embodiments in which grapefruit extract is provided, the extract may be used, for example, for antibacterial and antiseptic properties. In several embodiments, grapefruit extract possesses antioxidant properties and shrinks pores of the skin. In some embodiments, grapefruit extract is obtained from Citrus paradisi. In several embodiments, the extract is extracted from the cell wall, leaf, root, bark, fruit, seed, flower or other part of the organism. In some embodiments, rosemary extract is used. In some embodiments, grapeseed extract is used. An extract may be obtained, for example, by the technique of extraction or by in vitro plant cell culture.

In those embodiments in which Opuntia extract (such as Opuntia ficus-indica stem extract or prickly pear cactus extract) is provided, it may be used, for example, as an antioxidant, to increase photoprotection of the skin and impart anti-aging properties. In some embodiments, Opuntia ficus-indica stem extract is obtained from prickly pear. In several embodiments, they also possess hydrating properties. An extract may be obtained, for example, by the technique of extraction or by in vitro plant cell culture.

In those embodiments in which Sodium Lauroyl Sarcosinate is provided, it may be used, for example, as conditioning agents and surfactants. In some embodiments, derivatives of sarcosine, such as cocoyl sarcosine, lauroyl sarcosine, myristoyl sarcosine, oleoyl sarcosine, stearoyl sarcosine, sodium cocoyl sarcosinate, sodium myristoyl sarcosinate, ammonium cocoyl sarcosinate, and ammonium lauroyl sarcosinate are used.

In those embodiments in which Lactobacillus ferment is provided, it may be used, for example, as a probiotic ingredient. According to several embodiments, Lactobacillus ferment can function as an antioxidant activated by heat and light, to protect against UV damage, to preserve and reinforce the skin's natural defenses, and to improve epidermal structural integrity. In some embodiments, Lactobacillus ferment slows down the growth of harmful bacteria and maintains a balanced microbiome. In some embodiments, Lactobacillus ferment can be derived from cow milk. In some embodiments, Lactobacillus ferment is a product of the fermentation of Lactobacillus fermentum and is one non-limiting example of the Lactobacillus genus than can be used herein. Other species include but are not limited to L. acidophilus, L. casei, L. casei Shirota, L. fermentum, L. gasseri, L. helveticus, L. johnsonii, L. paracasei, L. plantarum, L. reuteri, L. rhamnosus, L. rhamnosus GG. In several other embodiments, Bifida Bacteria fermented Bifida ferment can be used as a probiotic. In some embodiments, rice ferment or sake ferment can be used as a probiotic. In some embodiments, a yeast derived ferment (e.g., Saccharomyces ferment) can be used as a probiotic. Fermented or non-fermented alternatives can be used. Gram-positive bacteria, including gram-positive, lactic acid producing bacteria are used in several embodiments. In some embodiments, the formulations include Lactobacillus species other than L. plantarum or its ferment (although this specific may be used in other embodiments). In some embodiments, a plurality of species of the same genus are used. In other embodiments, combinations of two or more genera are used (e.g., Lactobacillus and Bifidobacterium for example). In yet other embodiments, combinations of two or more different microbes are used (e.g., bacteria and yeast, for example).

Also provided for herein are methods for treating dehydrated or otherwise dry, unbalanced microbiome, and/or irritated skin, the methods comprising identifying at least one region of skin tissue exhibiting such symptoms, and applying, or instructing application of, a topical formulation to the skin region, wherein the formulation hydrates the dehydrated skin, balances the beneficial and harmful bacteria populations within the region of skin tissue, and provides improved epidermal barrier function, wherein the formulation comprises caprylic/capric triglyceride, cetyl palmitate, sorbitan stearate, a vitamin E compound (e.g., tocopherol), Lycium barbarum (Goji) fruit extract and Lactobacillus ferment.

Also provided for herein are methods for cleansing skin in a manner that primes the skin for being moisturized the methods comprising applying, or instructing application of, a topical formulation to the skin region, wherein the formulation cleanses the skin, primes the skin for a rebalancing or improvement in the balance of beneficial and harmful bacteria populations within the region of skin tissue, wherein the formulation comprises Sodium lauroyl sarcosinate, Citrus paradisi (Grapefruit) fruit extract and/or Opuntia ficus-indica stem extract, and Lactobacillus ferment.

In several embodiments, the topical formulation is a liquid, toner, essence, cream, serum, or gel. Depending on the embodiment, at least one of the Citrus paradisi (Grapefruit), Opuntia streptacantha, and Opuntia ficus-indica extract, and Lycium barbarum (Goji) extract is a stem extract. In several embodiments, the topical formulation decreases production of inflammatory mediators, improves the barrier function of the skin, leading to improved hydration and antioxidant production, and/or conditions the skin while providing protection from blue light and/or UV exposure.

### Hydrating Formulations

Despite the large number of cosmetic formulations on the market for hydration, there remains a need for a cosmetic formulation that can hydrate, improve and/or maintain the health of the skin, including maintenance of skin microbiome, stimulate Vitamin D or its precursor and/or has antiaging properties. Both healthy and damaged skin can benefit from several of the formulations described herein. Damaged skin can include skin with hyperpigmentation, UV damage, dry skin, oily skin, acne and/or rosacea. Several embodiments stimulate vitamin D production and/or activity. In some embodiments, vitamin D is stimulated (e.g., stored, synthesized, activated, released and/or otherwise enhanced) only in or at least in keratinocytes and fibroblasts. Several embodiments increase at least one precursor to vitamin D (such as lumisterol), which in turn may act as a vitamin D reservoir allowing for vitamin D metabolites to be stimulated even in low light or dark conditions. In one embodiment, the precursors are selectively increased in fibroblasts or other cells.

In some embodiments, the formulation comprises, consists essentially of or consists of several (e.g., 6, 8, 10) or all of the following groups of ingredients listed in Table 3 below that, for example, help cleanse the skin.

TABLE 4

| | | % in total formula in some embodiments as, for |
|---|---|---|
| Group | Example ingredients in each group | example, % m/m, % m/v, % w/w, % w/v or % v/v |
| Group 4.1 | water | about 20-90% (e.g., about 25, 35, 45, 55, 60, 65, 70, 75, |
| | | 80, 85, 90%) |
| Group 4.2 | at least two (or all of): water | about 1-7% (e.g., about 1, 2, 3, 4, 5, 6, 7%) as a group, |
| | propanediol, Lycium barbarum (Goji) | wherein individual ingredients are about 0.001-3% (e.g., |
| | fruit extract, gluconolactone, sodium | about 0.001, 0.002, 0.003, 0.004, 0.005, 0.01, 0.03 0.06, |
| | benzoate and calcium gluconate | 0.2, 0.5, 0.8, 1, 2, 3%) |
| Group 4.3 | At least two (or all of): water, Opuntia | about 0.1-4% (e.g., about 0.1, 0.5, 0.9, 1, 1.1, 1.5, 2, 3, |
| | ficus-indica stem extract or other | 4%) as a group, |
| | extracts of parts of the fruits of the | wherein individual ingredients are about 0.001-2% (e.g., |
| | genus Opuntia (including but not | about 0.001, 0.002, 0.003, 0.004, 0.007, 0.01, 0.02, |
| | limited to prickly pear cactus) Opuntia | 0.03, 0.06, 0.08, 0.09, 0.1, 0.2, 0.5, 0.7, 0.8, 1, 1.5, 2%) |
| | ficus-indica stem extract, glycerin, and | |
| | phenoxyethanol | |
| Group4.4 | at least two (or all of): water, sodium | about 0.1-4% (e.g., about 0.1, 0.5, 0.9, 1, 1.1, 1.5, 2, 3, |
| | chloride, hydrolyzed pea protein, | 4%) as a group, |
| | glucose, glycerin, and sodium | wherein individual ingredients are about 0.0001-3% (e.g., |
| | succinate | about 0.0001, 0.0002, 0.0005, 0.001, 0.005, 0.01, 0.02, |
| | | 0.03, 0.04, 0.05, 0.06, 0.08, 0.1, 0.4, 0.5, 0.7, 0.8, 1, 1.5, |
| | | 2, 3%) |
| Group 4.5 | at least one (or all of): water, sodium | about 0.01-4% (e.g., about 0.01, 0.1, 0.2, 0.4, 0.5, 0.9, 1, |
| | hyaluronate (which can be a single or | 1.1, 1.2, 1.5, 2, 3, 4%) as a group, |
| | different molecular weights include a bi | wherein individual ingredients are about 0.001-2% (e.g., |
| | and tri hyaluronate) and | about 0.001, 0.002, 0.009, 0.01, 0.02, 0.08, 0.1, 0.2, 0.4, |
| | phenoxyethanol | 0.7, 0.8, 0.9, 1, 1.5, 2%) |
| Group 4.6 | One or both of: Lactobacillus ferment | about 0.1-4% (e.g., about 0.5, 0.9, 1, 1.1, 1.5, 2, 3, 4%) |
| | and phenoxyethanol | as a group, |
| | | wherein individual ingredients are about 0.001-2% (e.g., |
| | | about 0.001, 0.002, 0.005, 0.007, 0.01, 0.02, 0.03, 0.06, |
| | | 0.09, 0.1, 0.2, 0.4, 0.5, 0.7, 0.8, 1, 1.5, 2%) |
| Group 4.7 | squalane | about 0.5-5% (e.g., about 0.5, 1, 1.5, 2, 2.5, 3, 3.5, 4, 4.5, |
| | | 5%) |
| Group 4.8 | polyglyceryl-10 oleate | about 0.5-10% (e.g., about 0.5, 1, 2, 3, 4, 5, 6, 7, 8, 9, |
| | | 10%) |
| Group 4.9 | at least two or all of water, | about 0.1-4% (e.g., about 0.5, 0.9, 1, 1.1, 1.5, 2, 3, 4%) |
| | glutamylamidoethyl imidazole | as a group, |
| | pentylene glycol and | wherein individual ingredients are about 0.001-2% (e.g., |
| | sodium benzoate | about 0.001, 0.002, 0.005, 0.007, 0.01, 0.02, 0.03, 0.06, |
| | | 0.09, 0.1, 0.2, 0.4, 0.5, 0.7, 0.8, 1, 1.5, 2%) |
| Group 4.10 | at least two (or all of): water, | about 0.01-4% (e.g., about 0.01, 0.1, 0.2, 0.4, 0.5, 0.9, 1, |
| | fructooligosaccharides, glycoproteins, | 1.1, 1.2, 1.5, 2, 3, 4%) as a group, |
| | bioflavonoids, glycerin, pentylene | wherein individual ingredients are about 0.001-2% (e.g., |
| | glycol, Cyamopsis Tetragonoloba | about 0.001, 0.002, 0.009, 0.01, 0.02, 0.08, 0.1, 0.2, 0.4, |
| | (Guar) Gum, xanthan gum and | 0.7, 0.8, 0.9, 1, 1.5, 2%) |
| | phytic acid | |
| Group 4.11 | Sodium hydroxide | about 0.1-4% (e.g., about 0.5, 0.9, 1, 1.1, 1.5, 2, 3, 4%) |
| Group 4.12 | at least two (or all of): phenoxyethanol, | about 0.01-4% (e.g., about 0.01, 0.1, 0.2, 0.4, 0.5, 0.9, 1, |
| | ethylhexylglycerin and/or tocopherol | 1.1, 1.2, 1.5, 2, 3, 4%) as a group, |
| | | wherein individual ingredients are about 0.0001-2% (e.g., |
| | | about 0.0001, 0.001, 0.002, 0.009, 0.01, 0.02, 0.08, 0.1, |
| | | 0.2, 0.4, 0.7, 0.8, 0.9, 1, 1.5, 2%) |

In some embodiments, formulations according to Table 4 have a pH at 25 degrees Celsius of about 5.0-7.7 (e.g., 5.0-5.5, 5.5-6.0, 6.0-6.5, 6.5-7.0, 7.0-7.7)).

In the table above, the ranges provided in the parentheticals include the overlapping ranges between the numbers. For example, a range of 20-60% (e.g., about 25, 35, 45, 55%) includes, for example, ranges between 25-35%, 25-55%, 45-55%, 35-55%, etc.). In one embodiment, the percentages provided are % m/m or % w/w.

In one embodiment, a hydrating, anti-aging formulation, whether in a gel, spray, mist, foam, leave-on mask, or other topical form, is adapted for acne-prone skin and optionally includes one or more of the following additional ingredients: alpha-hydroxy acids (AHAs), beta-hydroxy acids (BHAs) including salicylic acid, sulfur, benzoyl peroxide, azelaic acid, niacinamide, retinoids (such as retinal, retinol, adapalene), and tea tree oil. In several embodiments, these additional ingredients are each provided in a range of about 0.05-10% of the total formulation (e.g., 0.05-1%, 0.5-1%, 1-2%, 2-3%, 3-5%, 5-10% and overlapping ranges therein). In one embodiment, an effective amount of an ingredient to treat acne is provided that is at least 1% of the formulation (e.g., 1-10%, 1-15%, or 1-20%).

An enhanced glow or radiance is achieved by several embodiments. Many formulations are not only therapeutic and protective, but also nourish the skin, increase hydration, and improve overall appearance, texture and firmness. Thus, several formulations of the invention are beneficial for skin that is aged, sun-damaged, wrinkled, lax, and/or blemished. Formulations according to several embodiments can be used anywhere on the body, and are especially beneficial for the face, neck, décolletage and hands, where signs of skin damage and aging may be particularly prominent.

In one embodiment, the formulation is configured for treatment as a hydrating spray and/or mist for the body. In several embodiments, the formulation can be used under makeup and/or sunscreen. In several embodiments, the formulation provides hydrating and/or moisturizing effects. In several embodiments, the formulation provides skin firmness. In several embodiments, the formulation has antiaging benefits. In several embodiments, the formulation stimulates Vitamin D and/or its precursor in skin cells. In several embodiments, the formulation promotes and/or maintains a balanced skin microbiome to support a healthy skin barrier. In several embodiments, the formulation provides hydrating and/or moisturizing effects without feeling heavy on the skin. In several embodiments, the formulation works at multiple levels of the skin to hydrate, smooth and soften rough texture, and plump appearance of fine lines.

In some embodiments, the invention comprises a topical formulation for hydrating and/or treating skin, such as hydrating and/or moisturizing dry, flaky, and/or itchy skin in a subject, wherein the topical formulation is a liquid (such as serum, mist, lotion, liquid, oil or cream), a gel, a spray, a powder, a jelly, or a combination thereof. Physical and/or chemical exfoliants are provided in some embodiments. AHAs, BHAs, or both are provided in some formulations, including cleansing and moisturizing formulations. Pads soaked with the formulations described herein are provided in some embodiments, while in other embodiments patches that can remain on the skin for several hours or overnight that are pre-treated with the formulations described herein are provided. Such patches include, but are not limited to, absorbent patches, hydrocolloidal patches, silicone patches, and hydrogel patches. Some patches are designed to penetrate below the skin surface for effective delivery of ingredients.

In addition, in some embodiments, at least one of the following ingredients is included: a skin conditioning agent, a solvent, a binder, an emollient, a preservative, a thickener, and an antioxidant. In several embodiments, the formulations are silicone and/or preservative-free. Colorants and fragrances may additionally be included. In some embodiments, the formulation further includes one or more of the following: amino acids, peptides, phospholipids, additional vitamins, growth factors, and additional anti-aging compounds. Surfactants, gelling agents, and pH balancers may also be included.

In one embodiment, formulations for hydrating skin (including on the scalp, face and body), comprise or consist essentially of: water, glutamylamidoethyl imidazole, pentylene glycol, a bacterial product (such as Lactobacillus ferment), fructooligosaccharides, glycoproteins, bioflavonoids, fruit extract (such as goji fruit extract), hydrolyzed pea protein, squalane, polyglyceryl-10 oleate and one or more ingredients selected from the group consisting or consisting essentially of propanediol, Lycium barbarum (Goji) fruit extract, gluconolactone, sodium benzoate, calcium gluconate, Opuntia extract (such as Opuntia ficus-indica stem extract or prickly pear cactus extract), glycerin, phenoxyethanol, glucose, sodium chloride, sodium succinate, sodium hyaluronate, Cyamopsis tetragonoloba (Guar) gum, xanthan gum, phytic acid, sodium hydroxide, ethylhexylglycerin and tocopherol.

For example, such formulations can include the following ranges of ingredients: water in the range of 60-95% (e.g., about 65-90%), hydrolyzed pea protein in the range of 0.0001-4% (e.g., about 0.0003-2%), squalane in the range of 0.1-4% (e.g., about 0.5-3%), Lactobacillus ferment in the range of 0.1-4% (e.g., about 0.5-3%), Glycerin in the range of 2-8% (e.g., about 2-7%), Lycium barbarum (Goji) fruit extract in the range of 0.001-2% (e.g., about 0.01-1%), Opuntia extract (such as Opuntia ficus-indica stem extract or prickly pear cactus extract) in the range of 0.001-2% (e.g., 0.001-1%) and one or more ingredients selected from the group consisting or consisting essentially of propanediol, sodium benzoate, glucose, sodium chloride, sodium succinate, sodium hyaluronate, phenoxyethanol, tocopherol, phenethyl alcohol, gluconolactone, calcium gluconate, polyglycerol-10 oleate, glutamylamidoethyl Imidazole, pentylene glycol, fructooligosaccharides, glycoproteins, bioflavonoids, Cyamopsis tetragonoloba (Guar) gum, xanthan gum, phytic Acid and ethylhexyl glycerin in a collective range of about 0.1-10% (e.g., about 2-8%).

In one embodiment, the formulation comprises or consists essentially of water in the range of 60-95% (e.g., about 65-90%), propanediol in the range of 0.1-3% (e.g., about 0.2-2%), glycerin in the range of 2-8% (e.g., about 2-7%), gluconolactone in the range of 0.01-5% (e.g., about 0.02-2%), calcium gluconate in the range of 0.01-5% (e.g., about 0.02-2%), hydrolyzed pea protein in the range of 0.0001-2% (e.g., about 0.0005-1%), Lycium barbarum (Goji) fruit extract in the range of 0.001-2% (e.g., about 0.01-1%), Opuntia extract (such as Opuntia ficus-indica stem extract or prickly pear cactus extract), Lactobacillus ferment in the range of 0.001-5% (e.g., about 0.001-3%), sodium benzoate in the range of 0.001-2% (e.g., about 0.001-1%), glucose in the range of 0.001-2% (e.g., about 0.001-1%), tocopherol in the range of 0.0001-2% (e.g., about 0.0005-1%), squalane in the range of 0.001-3% (e.g., about 0.001-2%), polyglyceryl-10 oleate in the range of 0.1-5% (e.g., about 0.2-3%), glutamylamidoethyl imidazole in the range of 0.001-5% (e.g., about 0.001-3%), pentylene glycol in the range of 0.001-5% (e.g., about 0.001-3%), fructooligosaccharides in the range of 0.01-5% (e.g., about 0.02-2%), glycoproteins in the range of 0.01-5% (e.g., about 0.02-2%), bioflavonoids in the range of 0.01-5% (e.g., about 0.02-2%), Cyamopsis tetragonoloba (Guar) gum in the range of 0.001-5% (e.g., about 0.001-3%), xanthan gum in the range of 0.001-5% (e.g., about 0.001-3%) and phytic Acid in the range of 0.001-5% (e.g., about 0.001-3%).

Optionally, one or more of the following ingredients are also included: vitamin D based compound, vitamin C based compound, vitamin K based compound, vitamin B12 based compound, Arnica extract (root, bark, seed or fruit extract), Centella Asiatica extract (root, bark, seed or fruit extract), copper, potassium, ceramides, a multi-mineral complex comprising sodium PCA (pyrrolidone carboxylic acid), zinc based compound such as zinc PCA, magnesium based compound, calcium based compound and manganese based compound. In some embodiments, the ceramides comprise or consist essentially of: ceramide NP, ceramide AP, ceramide EOP, phytosphingosine, cholesterol, water, sodium lauroyl lactylate, carbomer and xantham gum. Other ingredients that are optionally included comprise one or more of the following, which may be particularly helpful to reduce or treat acne: alpha-hydroxy acids (AHAs), beta-hydroxy acids (BHAs) including salicylic acid, sulfur, benzoyl peroxide, azelaic acid, niacinamide, retinoids (such as retinal, retinol, adapalene), and tea tree oil. The anti-acne agents are formulated and balanced within the formulation such that the healthy microbiome is still preserved in several embodiments. In one embodiment, a formulation adapted for acne-prone or hyperpigmentated skin is provided and further includes one or more of the following ingredients: salicylic acid, lactic acid, glycolic acid, sulfur, benzoyl peroxide, azelaic acid, niacinamide, retinoids (such as retinal, retinol, adapalene), tranexamic acid (e.g., cetyl tranexamate mesylate), alpha arbutin, and tea tree oil. In several embodiments, these additional ingredients are each provided in a range of about 0.001-10% of the total formulation (e.g., 0.001-0.01%, 0.01-0.05%, 0.05-1%, 0.5-1%, 1-2%, 2-3%, 3-5%, 5-10% and overlapping ranges therein). In one embodiment, an effective amount of an ingredient to treat acne is provided that is at least 1% of the formulation (e.g., 1-10%, 1-15%, or 1-20%).

In some embodiments, the formulation included one or more of propanediol, pentylene glycol, levulinic acid and glyceryl caprylate. In one embodiment, the formulation comprises or consists essentially of propanediol in the range of 0.1-3% (e.g., about 0.2-2%), pentylene glycol in the range of 0.001-4% (e.g., about 0.01-3%), levulinic acid in the range of 0.001-4% (e.g., about 0.01-3%) and glyceryl caprylate in the range of 0.001-4% (e.g., about 0.01-3%).

In one embodiment, the formulation comprises or consists essentially of a solvent, one or more conditioning agents, one or more of preservatives and/or biocides, emollients, humectants, viscosity increasing/decreasing agents, surfactants, conditioners, antioxidants, flavoring agents, emulsifiers, fragrances, chelators, absorbents, and pH adjusters. In some embodiments, the preservatives is antimicrobial. In some embodiments, the preservatives is antibacterial. In some embodiments, the preservatives is antifungal.

Several embodiments are particularly adapted to calm skin and comprise more than 50%, 75%, 85%, 90%, or 95% of the ingredients provided in the tables provided herein and additionally one or more of the following ingredients: niacinamide, titanium dioxide, zinc oxide, water, glycerin, C13-15 alkane, dimethyl isosorbide, triethylhexanion, caprylic/capric triglyceride, sorbitan stearate, polyglyceryl-2 diisostearate, disodium lauriminodipropionate tocopheryl phosphate, polyacrylate crosspolymer-6, bisabolol, sorbityl laurate, cetyl palmitate, Tremella fuciformis sporocarp (mushroom) extract, magnesium carboxymethyl beta-glucan, polysorbate 80, betaine, citric acid, Crithmum maritimum extract, t-butyl alcohol, hydrogenated lecithin, Magnolia officinalis bark extract, tocopherol, Zingiber officinale (ginger) root extract, glycolic acid, palmitoyl tetrapeptide-10, sodium benzoate, potassium sorbate, phenoxyethanol, benzoic acid, dehydroacetic acid and chloroacetic acid. In some embodiments, the formulation further comprises a marine plant extract from sea fennel. Such calming formulations, according to several aspects, include several or all of the following benefits: calms and soothes sensitive and irritated skin, skin is cooled upon application, provides a smooth texture where the skin's surface is balanced, uniform and luminous, restores the skin where the skin is deeply hydrated and barrier health is improved to maintain moisturization, diminishes the appearance of redness with continuous use, non-comedogenic, and hypoallergenic.

Several embodiments are particularly adapted to diminish hyperpigmentation and comprise more than 50%, 75%, 85%, 90%, or 95% of the ingredients provided in the tables provided herein and additionally one or more of the following ingredients: titanium dioxide, zinc oxide, cyclopentasiloxane, isocetyl stearoyl stearate, dimethicone crosspolymer, Thermus thermophillus ferment, water, hydrated silica, dimethicone/vinyl dimethicone crosspolymer, disodium lauriminodipropionate tocopheryl phosphates, panthenyl triacetate, rheum extract (such Rheum rhaponticum root extract), Bidens pilosa extract, Elaeis guineensis (palm) oil, Gossypium herbaceum (cotton) seed oil, Linum usitatissimum (linseed) seed oil, tocopherol, dimethiconol, Citrus paradisi (grapefruit) seed extract, glycerin, dimethicone, fusanus spicatus wood oil, Vanilla planifolia fruit extract, ascorbic acid, caprylic/capric triglyceride, pentylene glycol, triethoxycaprylylsilane, acrylates/c12-22 alkyl methacrylate copolymer, phenoxyethanol, benzoic acid, dehydroacetic acid, potassium sorbate, farnesol and iron oxides. Such pigment correcting formulations, according to several aspects, include several or all of the following benefits: provides a broad spectrum sun protection, reduce skin discoloration, water resistant, non-comedogenic, paraben-free and gluten free.

In several embodiments, the water used in the formulation is one or more of distilled, deionized, and/or sea water.

In several embodiments, the formulation comprises a humectant. Phytic acid can be used as a humectant for several embodiments. In several other embodiments, betaine, glycerin and/or glucose can be used as a humectant. In several other embodiments, urea, hyaluronic acid, salicylic acid, alpha hydroxy acids (AHAs), such as glycolic acid and lactic acid, propylene glycol, Panthenol, honey or sorbitol can be used as a humectant.

In several embodiments, the formulation comprises a skin-conditioning agent. Cocamidopropyl Hydroxysultaine, Cocamidopropyl Betaine, glycerin, Glycereth-18 Ethylhexanoate, Opuntia extract (such as Opuntia ficus-indica stem extract or prickly pear cactus extract) Opuntia ficus-indica stem extract can be used herein as a skin-conditioning agent. In several other embodiments, triheptanoin, tricaprin, Camellia seed oil, cetyl alcohol, kokum, almond oil, petroleum jelly, mineral oil, castor oil, argan oil, shea butter, cocoa butter, beeswax or lanolin can be used as a skin-conditioning agent.

In several embodiments, the formulation comprises a binder and/or an emulsion stabilizer. Cyamopsis Tetragonoloba (Guar) Gum can be used herein as a binder and/or an emulsion stabilizer. In some embodiments, xanthum gum can be used herein as a binder and/or an emulsion stabilizer. In several other embodiments, acacia gum, alginates, certain fibers, flaxseed powder, cellulose gums or carrageenan can be used as a binder and/or an emulsion stabilizer.

In those embodiments in which Opuntia extract (such as Opuntia ficus-indica stem extract or prickly pear cactus extract) is provided, it may be used, for example, as an antioxidant, to increase photoprotection of the skin and impart anti-aging properties. In some embodiments, Opuntia ficus-indica stem extract is obtained from prickly pear. In several embodiments, they also possess hydrating properties. An extract may be obtained, for example, by the technique of extraction or by in vitro plant cell culture.

In those embodiments in which a plant sugar, fructooligosaccharides is provided, it may be used, for example as a humectant. In some embodiments, fructooligosaccharides can be derived from plants such as Lithops pseudotruncatella. In some embodiments, other plant sugars such as alpha-glucan oligosaccharide is used. In some embodiments, the plant sugars promote absorption and retention of water in the skin, which promotes vitamin D synthesis (or otherwise stimulates vitamin D and/or a vitamin D precursor or other vitamin D based compound). In some embodiments, betalain compounds are used instead of lithops or can be optionally be used as an extract from lithops.

In those embodiments in which Lactobacillus ferment is provided, it may be used, for example, as a probiotic ingredient. According to several embodiments, Lactobacillus ferment can function as an antioxidant activated by heat and light, to protect against UV damage, to preserve and reinforce the skin's natural defenses, and to improve epidermal structural integrity. In some embodiments, Lactobacillus ferment slows down the growth of harmful bacteria and maintains a balanced microbiome. In some embodiments, Lactobacillus ferment can be derived from cow milk. In some embodiments, Lactobacillus ferment is a product of the fermentation of Lactobacillus fermentum and is one non-limiting example of the Lactobacillus genus than can be used herein. Other species include but are not limited to L. acidophilus, L. casei, L. casei Shirota, L. fermentum, L. gasseri, L. helveticus, L. johnsonii, L. paracasei, L. plantarum, L. reuteri, L. rhamnosus, L. rhamnosus GG. In several other embodiments, Bifida Bacteria fermented Bifida ferment can be used as a probiotic. In some embodiments, rice ferment or sake ferment can be used as a probiotic. In some embodiments, a yeast derived ferment (e.g., Saccharomyces ferment) can be used as a probiotic. Fermented or non-fermented alternatives can be used. Gram-positive bacteria, including gram-positive, lactic acid producing bacteria are used in several embodiments. In some embodiments, the formulations include Lactobacillus species other than L. plantarum or its ferment (although this specific may be used in other embodiments). In some embodiments, a plurality of species of the same genus are used. In other embodiments, combinations of two or more genera are used (e.g., Lactobacillus and Bifidobacterium for example). In yet other embodiments, combinations of two or more different microbes are used (e.g., bacteria and yeast, for example).

Also provided for herein are methods for treating dehydrated or otherwise dry, unbalanced microbiome, and/or irritated skin, the methods comprising identifying at least one region of skin tissue exhibiting such symptoms, and applying, or instructing application of, a topical formulation to the skin region, wherein the formulation hydrates the dehydrated skin, balances the beneficial and harmful bacteria populations within the region of skin tissue, and provides improved epidermal barrier function, imparts anti-aging benefits and promotes vitamin D synthesis (or otherwise stimulates vitamin d and/or a vitamin D precursor).

Also provided for herein are methods for treating skin in a manner that primes the skin for being moisturized the methods comprising applying, or instructing application of, a topical formulation to the skin region, wherein the formulation hydrates the skin, primes the skin for a rebalancing or improvement in the balance of beneficial and harmful bacteria populations within the region of skin tissue.

In several embodiments, the topical formulation is a liquid, mist, spray, toner, essence, cream, serum, or gel. In several embodiments, the topical formulation decreases production of inflammatory mediators, increases the production of vitamin D and its precursors, improves the barrier function of the skin, leading to improved hydration and antioxidant production, and/or conditions the skin while providing protection from blue light and/or UV exposure.

### Skin Care Formulations as Described Generally Herein

Several of the ingredients below may be applicable to the formulations described herein for skin rehydration (e.g., face and surrounding regions, other body parts), balancing of the skin microbiome, improved skin barrier function, skin renewal, treating hyperpigmentation, and/or soothing redness correction.

In several embodiments, use of the formulations described herein reduces skin damage by about 10-100% (e.g., about 10%, 25%, 50%, 75%, 100% and ranges in between) after use. For example, significant improvements in skin pore size, fine lines, overall appearance, radiance, skin smoothness, and/or skin tone (evenness), and/or improved skin health (reduced itchiness, flaking etc.) are visible in several embodiments after 2 weeks, 4 weeks, 8 weeks or 12 weeks. Certain improvements in skin may be visible or felt within days of use. In several embodiments, the formulations provided for herein reduce and/or ameliorate one or more symptoms of irritation of skin (including itching or burning) and or reduce one or more symptoms of eczema, acne and/or rosacea.

In some embodiments of the invention, the anti-inflammatory agents decrease redness and irritation and fortify resistance to inflammation. The anti-inflammatory agents may have antioxidant properties by reacting with reactive oxygen species. The anti-inflammatory agents may also absorb the energy from blue light and/or UV light and are photo-protective and reduce blue light and/or UV-induced free radical damage to skin.

Although preservatives are provided in certain embodiments, the formulations described herein can be manufactured with reduced or no synthetic preservatives. In some embodiments, the formulations are free of one or more of the following: parabens, phthalates, sulfates, mineral oil, gluten, allergens, and irritants.

The agents, ingredients and compounds described herein may be modified natural substances that for example are not naturally occurring (e.g., isolates, extracts, lysates, ferments, purified, processed, chemically modified, etc.) or synthetic substances. Methods of using unique combinations of natural substances that do not exist in nature as that particular combination are also provided. An extract, isolate, lysate, etc., may be obtained, for example, by the technique of extraction or by in vitro plant cell culture.

In several embodiments, the invention comprises a method of treating skin using any one of the formulations described above or below. The use of any of the formulations described herein for treating skin, preventing skin damage, and/or improving skin appearance is provided in several embodiments. Several embodiments also include instructing the method or use of the formulation (e.g., via instructions for use).

The formulations described herein can provide both short-term and long-term benefits according to several embodiments. Beneficial effects from use of the formulations described herein occur upon use, within hours of use, within 1-2 days, 3-4 days, about 7 days, about 14 days, or within about 3 weeks. In several embodiments, the skin characteristics continue to improve over about 2-4 weeks, about 4-6 weeks, about 6-10 weeks, or about 12-16 weeks of use.

In some embodiments, the formulations described herein can be used as leave in formulations on the skin or hair or can be washed off after application. Further, the formulations can be applied on a cleansed skin or a non-cleansed skin. The time between the application and washing off the formulation can be within an hour of use, within 2 hours, 3 hours, about 5 hours. In several embodiments, the formulations provided for herein are suitable for 1, 2, or 3× daily topical application. In one embodiment, the formulation is left on overnight to provide benefits. In several embodiments, the formulations are able to be used on wet or dry hair or scalp. In several embodiments, the formulations are suitable for application to the scalp or hair and can be left on the scalp or hair until a subsequent shower/bath/wash, etc.

In some embodiments, after the formulation is applied to the skin, the formulation can be removed from the skin with water. In certain embodiments, the formulations can be removed without water, which can be accomplished by massaging the cleanser onto the skin, and, for example, wiping any excess formulation with a dry or wet cloth, paper towel or facial tissue. In several embodiments, the formulations are meant to be applied to the skin, or hair, but not purposefully removed (e.g., a “leave in” formulation).

According to several embodiments, the formulations described herein can be applied by hand, by sponge, by spraying, by applicator, such as a dropper, by brush, or through use of a wipe, pad, patch or tissue pre-treated or impregnated with ingredients described herein.

In some embodiments of the invention, the anti-inflammatory agents decrease redness and irritation (such as that caused by UV exposure) and fortify resistance to inflammation. The anti-inflammatory agents may have antioxidant properties by reacting with reactive oxygen species. The anti-inflammatory agents may also absorb the energy from UV light and are photo-protective, and reduce UV-induced free radical damage to skin. In several embodiments, the anti-inflammatory agents, by acting through a different pathway that involves inflammation's influence on pigmentation are particularly potent when combined with the anti-melanin agents. Post-inflammatory hyperpigmentation or hypermelanosis, which can occur after cutaneous inflammation or injury, is one example of hyperpigmentation that is related to inflammation, and according to several embodiments, is treated with the formulations described herein. Anti-inflammatory agents used in the formulations described herein include but are not limited to one or more of the following: vitamin E compounds (such as disodium lauriminodipropionate tocopheryl phosphate and other tocopherols), vitamin A compounds, vitamin B compounds, and vitamin D compounds. In several embodiments, the anti-inflammatory agent comprises a vitamin E compound (e.g., disodium lauriminodipropionate tocopheryl phosphate). The anti-inflammatory agents are provided, in several embodiments, in a range of about 0.1% to about 20% (e.g., 0.1%, 0.5%, 0.8%, 1%, 5%, 10%, 20%, and ranges in between) % m/m, % m/v, or % v/v in the formulation. In many embodiments, the percentages provided are % m/m or % w/w.

In some embodiments, the formulation comprises or consists essentially of one or more anti-melanin agents (such as tyrosine inhibitors), one or more anti-inflammatory agents, and one or more sun protection agents and at least one of the following ingredients: a skin conditioning agent, a solvent, a silicone, an alkane (e.g., a coconut or other plant-derived alkane), an emollient, a preservative, a thickener, an antioxidant, and an excipient. Optional colorants and fragrances may additionally be included. Agents to adjust or balance pH may also be included.

The use of agents, ingredients and compounds may be used interchangeably herein. Reference to the term “based” includes the recited agent, ingredient or compounds, including its derivatives, analogues, and pro versions. For example, a panthenol-based compound includes panthenol itself. The terms composition and formulation can be used interchangeably. Where percentages are provided for agents, ingredients and compounds, they can be % m/m, % m/v or % v/v with respect to the formulation as a whole, unless otherwise indicated,

In several embodiments, the invention comprises a method of treating skin using any one of the formulations described above or below. The use of any of the formulations described herein for treating skin (e.g., improving hydration and balancing the microbiome) and/or improving skin appearance is provided in several embodiments. Several embodiments also include instructing the method or use of the formulation (e.g., via instructions for use).

As described above, several embodiments of the present invention relate to unique skin care formulations for treating and protecting skin. The formulations described herein can be beneficial for both healthy and damaged skin. In several embodiments, use of the formulations described herein provides one or more of the following advantages: (i) increased skin hydration (in some embodiments this also results in a reduction in the appearance of redness hyperpigmentation (e.g., reduction in age spots, discolored scar tissue, birthmarks, or other discoloration), (ii) improved balance and health of the skin microbiome, (iii) skin looks and feels smoother; (iv) increased firmness, (v) increased hydration, (vi) improved skin tone and texture (e.g., increased evenness), (vii) clearer complexion, (viii) improved radiance, (ix) fine lines, pores, and wrinkles appear less visible, (x) improved overall appearance of skin, (xi) reinforcement of the skin's natural defenses, and (xii) improved epidermal structural integrity. Advantageously, in several embodiments, these improvements are obtained with formulations that are gentle and non-irritating, with no or little erythema, edema, dryness, peeling, itching, stinging, tingling, or burning sensation. Because undesired effects are nominal or nonexistent, the formulation fosters regular use by a subject, which enables longer term improvements in skin characteristics.

The formulations described herein can provide both short-term and long-term benefits according to several embodiments. Beneficial effects from use of the formulations described herein occur upon use, within hours of use, within 1-2 days, 3-4 days, about 7 days, about 14 days, or within about 3 weeks. In several embodiments, the skin characteristics continue to improve over about 2-4 weeks, about 4-6 weeks, about 6-10 weeks, or about 12-16 weeks of use.

Several embodiments of the invention comprise a formulation for protecting against UV damage and reinforcing the skin's natural defenses. In some embodiments, the formulation is in a liquid, gel or powder form. The formulations described herein can be applied prior to or after foundation or other make-up. In some embodiments, the formulation is colorless; however, in other embodiments, the formulation contains sufficient color to serve as foundation or cover-up. In some embodiments, the pH of the formulations is slightly basic, slightly acidic or neutral. In some embodiments, a pH of 3-5, 4-6, 5-7, 6-8, or ranges in between, are provided. Lower or higher pH values may also be used.

The formulations described herein have one or more of the following uses: primer, moisturizer, sunscreen, setting mist, and color (cover-up, coverage, or foundation). The unique aspects of many of the formulations described herein provide a multi-functional product that blends skin care and sun care, and offers a high SPF primer, color coverage, skin nourishment, anti-oxidants and a lightening effect (e.g., through a reduction of discoloration or pigmentation).

According to several embodiments, the formulations described herein can be applied by hand, by sponge, by spraying, by applicator, by brush, or through use of an impregnated pad, patch, wipe or tissue that is pre-treated with ingredients described herein. In some embodiments, the formulations are absorbent (e.g., readily absorbable) such that no separate means are needed to enhance absorption. However, in some embodiments, low frequency ultrasound, massage, application of an electrical field, mechanical manipulation or vibration may be used to facilitate absorption. In some embodiments, the formulation is useful post-surgery or dermatological treatment, where, for example, discoloration, may be an issue. Several topical formulations herein described can penetrate top layers of the skin. Further, some of the formulations described herein may be suitable for use for application under one or more skin layers (e.g., as an injectable).

Glyceryl isostearate is a type of glyceryl monoester that can be used herein. Glyceryl isostearate can be used as a skin conditioning agent and an emollient. In several embodiments described herein, the topical composition can comprise an emollient. In several embodiments described herein, the topical composition can comprise a skin conditioning agent. In several embodiments described herein, the topical composition can comprise a glyceryl monoester. Glyceryl monoesters that can be used in skin care and treatment can also include, but are not limited to, glyceryl laurate, glyceryl laurate, glyceryl laurate/oleate, glyceryl adipate, glyceryl alginate, glyceryl arachidate, glyceryl behenate, glyceryl caprate, glyceryl caprylate, glyceryl caprylate/caprate, glyceryl citrate/lactate/linoleate/oleate, glyceryl cocoate, glyceryl collagenate, glyceryl erucate, glyceryl hydrogenated rosinate, glyceryl hydrogenated soyate, glyceryl hydroxystearate, glyceryl isopalmitate, glyceryl isostearate, glyceryl isostearate/myristate, glyceryl isostearates, glyceryl lanolate, glyceryl linoleate, glyceryl linolenate, glyceryl montanate, glyceryl myristate, glyceryl isotridecanoate/stearate/adipate, glyceryl oleate, glyceryl oleate/elaidate, glyceryl palmitate, glyceryl palmitate/stearate, glyceryl palmitoleate, gyceryl pentadecanoate, glyceryl polyacrylate, glyceryl rosinate, glyceryl sesquioleate, glyceryl/sorbitol oleate/hydroxystearate, glyceryl stearate/acetate, glyceryl stearate/maleate, glyceryl tallowate, glyceryl thiopropionate, and glyceryl undecylenate.

Polyhydroxystearic acid is a suspending agent and an emulsifier that is used to stabilize products. It can be used herein to suspend SPF protection components in lotions, liquids and gels.

Decamethylcyclopentasiloxane is a cyclopentasiloxane, a silicone fluid that can be used herein, which can work as an emollient for the skin. However, in several embodiments, other emollients are used instead of cyclopentasiloxane. In several embodiments, dimethicone and/or alkane-containing ingredients are used, such as dimethicone crosspolymer, dimethicone and polysilicone-11 blend, and/or coconut (or other plant or synthetic source) alkane-dimethicone blend. In several embodiments, isododecane is used as a emollient. Trimethylsiloxysilicate can be used for an antifoaming agent, an emollient, and/or for conditioning the skin. In some embodiments, trimethylsiloxysilicate is a cross-linked silicone resin with film-forming attributes. It can hold pigments in place while providing water-resistance in some embodiments. In some formulations herein, it provides a long-lasting effect. In several embodiments, trimethylsiloxysilicate may reduce the number of times the formulation needs to be applied. Trimethylsiloxysilicate is a siloxane polymer. Other examples of siloxane polymers that can be used herein include silica silylate, silica dimethyl silylate, and trifluoropropyldimethyl/trimethylsioxysicate. In some embodiments, they are insoluble in water and used for film forming, wear and water resistance. In several embodiments described herein, the formulation comprises a siloxane polymer. In several embodiments, cyclomethicone and/or cyclopentasiloxane are not used.

Glycerin is also known as glycerol, or glycerine. It is a viscous liquid used in pharmaceutical formulations and cosmetic formulations. Glycerin comprises a glycerol backbone, and is central to all lipids and commonly known as a triglyceride. Several types of triglycerides can be used herein. These can be for example, caprylic/capric triglycerides, triglycerides with C10-C18 fatty acid chains, and triglycerides with C18-C36 fatty acid chains. Fatty acids can include for example, naturally occurring fatty acids which can vary in chain length from 6 to 24 carbon atoms, and can include both saturated and unsaturated fatty acids containing one or more double bonds, and other fatty acids that are known to those skilled in the art. Triglycerides are used herein in some embodiments as an emollient for the skin.

As described herein, anti-inflammatory agents other than vitamin E compounds can be used in addition to, or in lieu of, vitamin E compounds. Anti-inflammatory agents are used, in some embodiments, to inhibit the inflammatory pathway that can affect melanin. For example, during the inflammatory process in the epidermal layer, inflammatory mediators (such as cytokines, chemokines, prostanoids such as prostaglandins, reactive oxygen species, etc.) may be released. The inflammatory mediators, in turn, may stimulate melanocytes to affect the production, sequestering, and/or processing of melanin in a manner that contributes to hyperpigmentation. An anti-inflammatory agent, as used herein in several embodiments, interrupts (e.g., or otherwise inhibits) this pathway, which in turn helps to reduce hyperpigmentation. In some embodiments, the balancing of inflammation and inflammatory pathways is accomplished.

The topical formulations described herein may be used as a primer. In addition, according to several embodiments, the formulations may be foundation, blush, lip color, eye color, lotions, creams, serums, gels, cleansing formulations, eye creams, sunscreens, bronzers, powders, nail care, hair care, and other cosmetics and skin care products.

According to several embodiments, the ingredients may be delivered in a single formulation or separately. For example, the moisturizing agent and the prebiotic agent and probiotic (e.g., bacterial ferment) can be in a single formulation. Alternatively, the ingredients or groups of ingredients may be provided in separate compositions. For example, the moisturizing agent(s) and the prebiotic agent(s) may be in one formulation and the probiotic in another, or the moisturizing agent(s) and the probiotic agent(s) in one formulation and the postbiotic agent(s) in another. The topical formulations are, according to several embodiments, applied once a day, twice a day, or every other day. Greater frequencies may also be used. Lesser frequencies may also be used, for example in maintenance phase after the desired effects have been achieved. Multi-chamber dispensers can be used in some embodiments. In several embodiments, a kit comprising a formulation and one or more skin care or cosmetic products is provided. In one embodiment, the kit comprises the moisturizing and prebiotic agents in one unit and the probiotic separately. In one embodiment, the kit comprises the moisturizing and prebiotic agents in one formulation and, optionally, a separate probiotic. Applicators (brushes, sticks, sponges, etc.) may be provided to apply the formulations described herein, and may also be included in a kit. The kit can comprise one or more of the formulations described herein in varying strengths (e.g., of the active ingredients). The kit can comprise one or more of the formulations described herein as well as an addition sunscreen and/or exfoliant. In some embodiments, the formulations described herein can be applied by transdermal patch.

In several embodiments, skin hydration is increased by 10-100% after use. Additional improvements in skin barrier function, microbiome health, pore size, fine lines, overall appearance, radiance, skin smoothness, and/or skin tone (evenness) are visible in several embodiments after 4 weeks, 8 weeks and 12 weeks. Certain improvements in skin may be visible or felt upon use or within days of use. Although specific regions of skin requiring additional moisture are treated according to several embodiments, an overall hydrating effect can also be achieved on skin that has no identifiable regions of dryness or flaking.

Several embodiments of the formulations are particularly advantageous because they provide hydration and microbiome balance in a formulation that goes on smoothly and is not chalky or sticky. This is helpful to moisturize the skin while the formulation is simultaneously working to balance the microbiome and improve the barrier function of the skin (which in turn aids in retaining moisture in the skin). This is also helpful to minimize the number of products a user applies to his/her face (or body).

Several embodiments of the formulations are water resistant. In one embodiment, the formulation is water resistant, e.g., with respect to SPF, for up to 30, 40, 60 and 120 minutes. Long-wear formulations are provided in several embodiments. In several embodiments, the skin is trained to develop its own blue light and/or UV light resistance through use of the formulations described herein for several days, weeks or months, but without an SPF such as titanium dioxide, zinc oxide, oxybenzone, avobenzone, homosalate, octinoxate, octocrylene, or octisalate. An SPF of 15, 30, 50, or higher is provided in several embodiments. Some formulations do not use titanium dioxide or zinc oxide, while alternate formulas do include those ingredients. In one embodiment, a physical or chemical sunscreen is used.

In many embodiments, the reduction of inflammation through the inclusion of prebiotic agent(s) and probiotic agent(s) and the resultant postbiotic effects synergistically work with the other ingredients to promote epidermal integrity while supporting skin's good bacteria for a stable microbiome and improved skin health. Inflammation and irritation that is ameliorated by several embodiments herein may have been caused by, for example, an immune response (e.g. allergy), genetics, the aging process, or environmental causes (such as UV exposure).

Several embodiments disclosed herein provide for comprehensive skin care formulations that target one or more inflammatory mediators, thereby serving as anti-redness and anti-irritants and providing soothing, healing, or otherwise soothing effects for sensitive, reddened, or otherwise irritated skin. In several embodiments, the formulations improve the barrier function of the skin through stabilizing the microbiome and providing protection and/or enhancement of epidermal lipids, boosts cellular respiration and restores the water balance. Several embodiments improve the radiance of damaged skin, and in some embodiments also improve radiance and appearance of normal skin. In several embodiments, the formulations provided rebalance the skin's immune system, which leads to soothing of irritated, itching or otherwise sensitive skin.

In some embodiments, a multi-modal approach effectively soothes the skin. In one embodiment, hydration of the skin is increased by at least 10-90% post treatment with a formulation as described herein used consistently after 7, 14, 21, 30, 60 or 90 days. In one embodiment, barrier function and/or integrity of the epidermal layer of the skin is increased by at least 10-90% post treatment with a formulation as described herein used consistently after 7, 14, 21, 30, 60 or 90 days. In one embodiment, skin microbiome stability is increased by at least 10-90% post treatment with a formulation as described herein used consistently after 7, 14, 21, 30, 60 or 90 days. In one embodiment, inflammation is reduced by at least 10-90% post treatment with a formulation as described herein used consistently after 7, 14, 21, 30, 60 or 90 days. In one embodiment, cytokines are reduced by at least 10-90% post treatment with the formulation used consistently after 7, 14, 21, 30, 60 or 90 days. In one embodiment, skin irritation is reduced by at least 10-90% post treatment with the formulation used consistently after 7, 14, 21, 30, 60 or 90 days. In one embodiment, skin redness is reduced after 1-3 uses of the formulation by at least 10-75%. Wound healing, such as micro-wounds and abrasions, is also improved in one embodiment by at least 10-90% post treatment with the formulation used consistently after 7, 14, 21, 30, 60 or 90 days.

In some embodiments, the formulations provided herein are concentrated and provided in a mask form to be left on the skin for at least 5, 15, 30, or 60 minutes (or as an overnight night repair mask) for accelerated results. Optionally, the mask may include ingredients such as camphor, almond extract, honey, kaolin, basil, turmeric, and/or kelp. The non-mask formulations may also include camphor, almond extract, honey, kaolin, basil, turmeric, and/or kelp. Gentle cleansers may also include many of the ingredients of the formulations described herein and may additionally include witch hazel or other toners (for toner-type cleansers) or foaming ingredients (for washes and foaming cleansers). Concentrated spot treatments are provided in some embodiments. In one embodiment, the amounts of the ingredients are concentrated by 10% or more (as compared to the non-masks) and provided as a mask.

In some embodiments, visible reductions in the appearance of skin dryness and inflammation may be observed within minutes of use of a formulation as described herein, due to, for example, the hydrating agent(s) and/or the anti-inflammatory activity of the ingredients. A soothing or cooling sensation may be experienced by the user upon application to the skin.

In some embodiments, the formulations for treating skin redness and/or irritation are used during or after a dermatological procedure (including but not limited to brow lifts, blepharoplasty, botulin and other neurotoxins, facials, fillers, dermabrasion, microdermabrasion, micro-needling, peels, exfoliations, suctioning, fluid delivery, acid treatments, massage, extractions, energy-based and other treatments, such as lasers, thermal, radiofrequency, light (e.g., photofacials/IPL), etc.).

In several embodiments, the formulation is paraben-free and is configured for use on the face or body. In several embodiments, the formulation is a free from animal products (e.g., the components of the formulation are plant-based or synthetic). In several embodiments, the formulations provided for herein are suitable for daily topical application, and rated at Protection Grade of Ultraviolet A (PA) of 12-14, 14-16, 16-18 or greater (or any grade in between those listed) in a Persistent Pigment Darkening (PPD) test.

In several embodiments, there is also provided a use of a formulation disclosed herein for the treatment or prevention of dry, irritated or inflamed skin. In several embodiments, the use of the formulations disclosed herein quickly hydrates and repairs damage or inflammation on skin. In several embodiments, use of the formulations helps to soothe and calm sensitive skin, while, in several embodiments, providing protection from blue light and/or sun exposure. In several embodiments, the formulation provides ongoing hydration and improved skin barrier functionality. In several embodiments, formulations provided for herein result in one or more of: harmonizing epidermal maturation, improved skin uniformity, clear and glowy skin appearance, molecular, cellular, and/or epigenetic regulation of skin, increase moisturization, skin smoothness, upregulate laminin, downregulate matric metalloproteinases (e.g., MMP1), antioxidant functionality, anti-inflammatory functionality, free radical quenching, promotes beneficial bacteria growth/health while discouraging harmful bacteria growth/health, induction of one or more beta-defensins (e.g., human β-defensin 3 (hBD-3)).

In several embodiments, the formulations provided for herein function to alleviate one or more pathways that can contribute to loss of skin hydration and/or skin barrier function. For example, mechanical factors may cause loss of skin hydration and/or skin barrier function in some embodiments. Additionally, physical and/or chemical factors may also play a role in development of loss of skin hydration and/or skin barrier function, either alone or in combination with mechanical factors. Non-limiting examples of mechanical factors that can cause, contribute to or are associated with loss of skin hydration and/or skin barrier function include mechanical hair removal, such as shaving or waxing, scrapes, abrasions (e.g., first, second or third degree), heat or drying effects from external sources, such as a hair dryer, or other mechanical means that create friction against the skin and can damage or injure one or more skin layers. In some embodiments, physical factors work in conjunction with mechanical factors to lead to loss of skin hydration and/or skin barrier function. Physical factors include, but are not limited to, UV exposure (e.g., sunlight), varied temperatures—whether hot or cold, either of which can cause an imbalance in skin health, other environmental factors such as humidity, and the like. For example, a cold environment can cause drying of skin, in part because of lower humidity, with the drying of the skin leading to cracking or other skin damage/loss of hydration. Similarly, elevated temperatures or high humidity can cause excessive perspiration or greater than normal moisture on skin surfaces, which can then lead to development of frication and/or skin abrasions, which as discussed above can lead to loss of skin hydration and/or skin barrier function. Chemical factors, which may work in combination with physical and/or mechanical factors, can also cause skin loss of skin hydration and/or skin barrier function. Chemical factors include, but are not limited to, certain sunscreens or other topical compositions (e.g., lotions, facial masks etc.), chemical hair removal agents, retinol (e.g., retinoid skin creams), exposure to preservatives (e.g., those in certain topical formulations).

In several embodiments, mechanical, physical, or chemical factors (or combinations of any or all of such factors) that can affect skin negatively are treated by the formulations described herein; for example one or more of these factors can initiate or otherwise promote skin dehydration, imbalance in the skin microbiome or loss of barrier function through damage or injury to the outer layers of skin, which normally function as a protective barrier. In some embodiments, these factors can cause a loss of natural or endogenous moisturizing factors, which can lead to dryness, loss of skin hydration and susceptibility to other pro-damaging factors or environments. Loss of lipids (e.g., epidermal lipids in the stratum corneum) can further degrade the barrier function of the skin, rendering it more susceptible to other factors and possibly to infectious agents. Oxidizing chemicals or free radical generation can be induced, which further disrupts the skin microbiome and compromises barrier function. Pro-damaging factors can also lead to loss of, degradation of, or reduced production of certain skin proteins such as laminins and/or beta defensins. Various proteins serve to maintain the working structure of skin cells, maintain a tight and intact external skin layer, and generally promote and maintain the barrier function of skin.

At least one result of the damages to the skin or reduction in barrier function is the subsequent cascade of biochemical events that occur in deeper layers of the skin, such as the epidermis. In the epidermal layer, a series of proinflammatory reactions can occur as a result of mechanical, chemical and/or physical events. For example, various proinflammatory cytokines can be released (II-1alpha, leukotriene B4, tumor necrosis factor alpha, prostaglandin E2, etc.). In several embodiments, there are multiple cascades or layers of proinflammatory cytokines. In some embodiments, one or more of the proinflammatory cytokines are released from, for example, keratinocytes and/or mast cells. These proinflammatory cytokines can have wide ranging effects, from local infiltration of immune cells, to activator of downstream receptors, such as heat receptors (which later impact neural sensory pathways to further exacerbate skin irritation. Further, mast cells can also release mediators of itching sensations (e.g., histamine). These various proinflammatory cytokines can activate cognate receptors and induce a change in neural signals that lead to an increased sensation of itching, burning, skin tightness, heat, etc. Other pathways, such as proteolytic pathways may be induced. For example, induction of matrix metalloproteinases (MMPs), zinc-containing endopeptidases that are able to degrade various components of extracellular matrix (ECM) proteins, such as collagen, can disrupt skin structure and/or the local biochemical environment in a region of skin. Thus MMP induction can not only disrupt the underlying structure of the skin, which can alter permeability and thus dehydration, but may also shift the biochemical environment in a manner that disfavors beneficial bacterial populations, thus compounding the skin issues by disrupting the microbiome. Not only does the activation of these pathways lead to the increased sensations of irritation, the increased sensations can lead to an individual exacerbating the problems (e.g., by itching the skin, further damaging the surface and generating more inflammatory mediator release), as well as additional dehydration of the skin.

Furthermore, there can result, in several embodiments, more long-term changes to the cells making up the various skin layers. For example, there can be alterations in proliferation and/or differentiation of cells in the skin. In one embodiment, keratinocytes, which serve as barrier cells, can over-proliferate which can lead to a coordinate excess of proinflammatory cytokine production and/or skin thickening. Likewise, changes in keratinocyte differentiation can be altered, thereby altering the balance between the various types of cells in the skin. These longer-term changes can result in a feed forward effect that leads to further skin susceptibility to the various factors that led to initial injury (e.g., mechanical, physical, chemical). As a result, the skin's natural barrier function can continue to degrade, which leads to dry, damaged, and sensitive skin—such characteristics being associated with increased chances for additional skin damage and irritation. Thus, mechanical, physical and/or chemical factors can create a cycle or loop of skin irritation, dehydration, dysfunctionality, and redness.

In several embodiments, the formulations described herein address (e.g., limit the effects of) one or more of these causes and/or symptoms of dehydrated, microbially imbalanced, damaged, irritated, or reddened skin. In several embodiments, the formulations described herein reduce the impact of mechanical factors on the skin and its barrier function. In several embodiments, the formulations provided for herein reduce the impact of chemical factors on the skin and its barrier function. In several embodiments, the formulations described herein reduce the impact of physical factors on the skin and its barrier function. In several embodiments, the formulations described herein limit release and/or effect of proinflammatory cytokines and/or other inflammatory mediators. In several embodiments, the formulations provided for herein limit the longer-term changes that could negatively impact skin, thereby limiting or reducing the feed-forward effect discussed above. In one embodiment, the formulations described herein maintain or enhance lipid production in the skin, for example, by stimulation of synthesis of certain ceramides, which are major components of the stratum corneum. In several embodiments, production of ceramides such ceramide III or VI, and/or enzymes that play a role in ceramide formation (e.g., beta-glucocerebrosidase) is elevated through use of the formulations described herein, which can enhance barrier function (and also moisture retention). In several embodiments, production or maintenance of other structural components of the skin, such as laminins (glycoproteins of the extracellular matrix) and/or filaggrin (monomers that are incorporated into skin lipids, thereby enhancing skin barrier function) is increased through use of the formulations described herein, which enhances the ability of the skin to resist negative impacts of mechanical, physical and/or chemical factors (and thus become less susceptible to irritants).

In still additional embodiments, the formulations described herein enhance one or more of skin cell regulation and overall skin protection. Cellular regulation of skin cells can be positively impacted by the formulations provided for, for example, by promoting proliferation of certain cells, such as epidermal basal cells. Also enhanced in several embodiments is cellular respiration. In conjunction, these effects result in a larger overall population of healthy skin cells, with each cell, or the majority of cells, undergoing greater regulation of cellular respiration to avoid or eliminate, for example, free radical production or other factors (e.g., production of proinflammatory cytokines) that could compromise skin function or integrity. Skin protective effects are also achieved through the use of formulations disclosed herein. For example, in several embodiments, the formulations regulate (e.g., reduce) proinflammatory mediators. Additionally, cellular stressors (e.g., free radicals resulting from oxidative/metabolic stress) are regulated (e.g., reduced to a normal, healthy level). In several embodiments, the formulations also provide an increased barrier function that allows the cells of the skin to become properly hydrated (thereby increasing the water reservoir of the skin) and also preventing or reducing loss of water through the skin, especially damaged skin.

The use of the terms agents, ingredients and compounds may be used interchangeably herein. In several embodiments, reference to the term “based” includes the recited agent, ingredient or compounds. For example, a bisabolol-based compound includes panthenol itself. As used herein, the terms composition and formulation can be used interchangeably. As used herein, the terms “skin care formulation(s)” and “cosmetic formulation(s)” can be used interchangeably. Where percentages are provided for agents, ingredients and compounds, they can be % m/m, % m/w, % w/w, % m/v, % v/v and variations thereof with respect to the formulation as a whole, unless otherwise indicated.

The agents, ingredients and compounds described herein may be modified natural substances (e.g., isolates, ferments, extracts, purified, processed, chemically modified, etc.) or synthetic substances. Methods of using unique combinations of natural substances are also provided.

In several embodiments, the invention comprises a method of treating skin using any one of the formulations described above or below. The use of any of the formulations described herein for treating skin (e.g., improving skin hydration and microbiome balance) and/or improving skin appearance is provided in several embodiments. Several embodiments also include instructing the method or use of the formulation (e.g., via instructions for use).

As described above, several embodiments of the present invention (e.g., cleansing and/or hydrating formulations) relate to unique skin care formulations for treating, protecting, and/or repairing skin. The formulations described herein can be beneficial for both healthy and damaged skin, for example skin that is irritated through aging, sun exposure, or other environmental stress. In several embodiments, use of the formulations described herein provides one or more of the following advantages: (i) reduction in the appearance of redness and/or blotchiness, (ii) reduction in skin irritation, aggravation, and/or discomfort, etc., (iii) skin looks and feels smoother; (iv) increased firmness, (v) increased hydration, (vi) improved skin tone and texture (e.g., increased evenness), (vii) clearer complexion, (viii) improved radiance, (ix) fine lines, pores, and wrinkles appear less visible, (x) improved overall appearance of skin, (xi) reinforcement of the skin's natural defenses, (xii) improved epidermal structural integrity (e.g., improvement of the skin's barrier function), (xiii) reduction or prevention of inflammation, (xiv) rebalancing of the skin's immune system, (xv) soothing of irritated skin, and (xvi) calming of sensitive and/or itchy skin. Advantageously, in several embodiments, these improvements are obtained with formulations that are gentle and non-irritating, with no or little erythema, edema, dryness, peeling, itching, stinging, tingling, or burning sensation. Because undesired effects are nominal or nonexistent, the formulation fosters regular use by a subject (even one with sensitive skin), which enables longer term improvements in skin characteristics.

The formulations described herein can provide both short-term and long-term benefits according to several embodiments. Beneficial effects from use of the formulations described herein occur upon use, within hours of use, within 1-2 days, 3-4 days, about 7 days, about 14 days, or within about 3 weeks. In several embodiments, the skin appears improved after application of the formulation and benefits continue over about 2-6 weeks, about 6-12 weeks, about 12-24 weeks, or about 24-52 weeks of use. In several embodiments, long lasting effects on the skin are achieved in less than 3 months. A formulation according to several embodiments provides an increase (e.g., by at least in skin moisture and skin barrier function (e.g., decrease in trans-epidermal water loss) immediately and/or after 4 weeks of use by at least 5% (e.g., 5%, 7.5%, 10%, etc.). Such parameters may be measured by Corneometer® and Tewameter® evaluations.

Several embodiments of the invention comprise a formulation for soothing irritated skin while also reinforcing the skin's natural defenses. In some embodiments, the formulation optionally includes ingredients that provide protection against UV damage. In some embodiments, the formulation is in a liquid, gel or solid (e.g., powder) form. The formulations described herein can be applied prior to or after foundation or other make-up. In some embodiments, the formulation is colorless; however, in other embodiments, the formulation contains sufficient color to serve as foundation and/or color corrector (e.g., with green undertones to balance redness). In some embodiments, the pH of the formulations is slightly basic, slightly acidic or neutral. In some embodiments, a pH of 3-5, 4-6, 5-7, 6-8, or ranges in between, are provided. Lower or higher pH values may also be used.

The formulations described herein have one or more of the following uses: cleanser, primer, moisturizer, skin protectant, sunscreen, setting mist, and color (cover-up, coverage, or foundation). The unique aspects of many of the formulations described herein provide a multi-functional product that blends skin care (e.g., skin repair) and sun care, and offers one or more of a high SPF primer, color coverage, skin nourishment, anti-oxidants and an anti-irritant/anti-inflammatory effect (e.g., through a reduction of inflammatory mediators, increasing cellular respiration, restoration of the skin's water balance).

According to several embodiments, the formulations described herein can be applied by hand, by sponge, by spraying, by applicator, by dropper, by brush, or through use of a composition-impregnated wipe or tissue. In some embodiments, the formulations described herein can optionally be applied by transdermal patch. In some embodiments, the formulations are absorbent (e.g., readily absorbable) such that no separate means are needed to enhance absorption. However, in some embodiments, one or more of low frequency ultrasound, massage, application of an electrical field, mechanical manipulation or vibration may be used to facilitate absorption. In some embodiments, the formulation is useful post-surgery or dermatological treatment, where, for example, skin irritation or damage may be an issue. Several topical formulations herein described can penetrate top layers of the skin. Further, some of the formulations described herein may be suitable for use for application under one or more skin layers (e.g., as an injectable or subcutaneous implant). Oral prebiotics, probiotics and/or postbiotics may be provided, for example, in addition to one or more topical formulations described herein to balance the microbiome both in the GI tract and on the skin (e.g., internally and externally).

Glyceryl compounds may be used, for example, as a skin conditioning agent and/or emollient include, but are not limited to glyceryl behenate, glyceryl laurate, glyceryl laurate, glyceryl laurate/oleate, glyceryl adipate, glyceryl alginate, glyceryl arachidate, glyceryl caprate, glyceryl caprylate, glyceryl caprylate/caprate, glyceryl citrate/lactate/linoleate/oleate, glyceryl cocoate, glyceryl collagenate, glyceryl erucate, glyceryl hydrogenated rosinate, glyceryl hydrogenated soyate, glyceryl hydroxystearate, glyceryl isopalmitate, glyceryl isostearate, glyceryl isostearate/myristate, glyceryl isostearates, glyceryl lanolate, glyceryl linoleate, glyceryl linolenate, glyceryl montanate, glyceryl myristate, glyceryl isotridecanoate/stearate/adipate, glyceryl oleate, glyceryl oleate/elaidate, glyceryl palmitate, glyceryl palmitate/stearate, glyceryl palmitoleate, glyceryl pentadecanoate, glyceryl polyacrylate, glyceryl rosinate, glyceryl sesquioleate, glyceryl/sorbitol oleate/hydroxystearate, glyceryl stearate/acetate, glyceryl stearate/maleate, glyceryl tallowate, glyceryl thiopropionate, and glyceryl undecylenate.

Polyhydroxystearic acid may be used in several embodiments, for example, as a suspending agent and/or an emulsifier that is used to stabilize products in some embodiments. It can be used herein to suspend SPF protection or other components in lotions, liquids, gels, etc.

Cyclopentasiloxane may be used in several embodiments, for example, as a silicone fluid that can work as an emollient for the skin. Depending on the embodiment, other silicone compounds may be used. For example, other examples of siloxane polymers that can be used herein (instead of or in addition to cyclopentasiloxane) include silica silylate, silica dimethyl silylate, polytrimethylsiloxymethacrylate copolymer, isododecane, acrylates and trifluoropropyldimethyl/trimethylsioxysicate. Other ingredients can function as emollients, such as dimethicone crosspolymer, cyclopentasiloxane, dimethicone and polysilicone-11 blend, and/or coconut (or other plant or synthetic source) alkane-dimethicone blend. In several embodiments, isododecane is used as an emollient. In some embodiments, these are insoluble in water and used for film forming, wear and water resistance. In several embodiments described herein, the formulation comprises a siloxane polymer. In several embodiments, silicones, including but not limited to cyclopentasiloxane, provide one or more of the following benefits, non-tacky, smooth feel, no oily after-feel, enhanced comfort during use, skin protection from particulate pollution, sebum absorption, enhanced durability of formulations, including increased color uniformity, improved wash-off resistance and duration of usability (e.g., for long-wear formulations). In some cases, where siloxanes are included such as cyclopentasiloxane, a by-product (such as cyclotetrasiloxane in one embodiment), or other emollients, may be present at, for example, 0.1%, 0.5%, 1%, 2%, 5%, and ranges in between. In several embodiments, cyclomethicone, cyclotetrasiloxane and/or cyclopentasiloxane are not used.

In those embodiments in which caprylic/capric triglycerides are provided, they may be used, for example, as skin conditioners and/or occlusive agents, the latter serving, in several embodiments, to increase the hydration of the skin (e.g., by preventing loss of moisture through the skin), thereby reducing the tendency of dry skin to become irritated. In some embodiments, caprylic/capric triglycerides are a mixed triester derived from coconut oil and glycerin. Caprylic/capric triglycerides function as a skin conditioning agent in several embodiments, helping to soften, smooth, and/or improve water barrier functions of skin. In several embodiments, they also function as emollient, dispersing agent and/or solvents. In several embodiments, the triglycerides provide a non-greasy barrier as well as skin lubrication. In several embodiments, their use can enhance delivery of ingredients contained in the formulations disclosed herein.

Preservatives such as benzoic acid (and its salt sodium benzoate), dehydroacetic acid, potassium sorbate and/or phenoxyethanol (and others) are used in many embodiments. In some embodiments, these preservatives are in amounts and/or are specifically chosen to be suitable for delicate or sensitive skin. Preservatives can be naturally occurring, modified, or synthetically produced. In addition to, or in lieu of the preservatives recited herein, the following may be used: fermented radish root, rosemary oleoresin extract, salicylic acid, sorbic acid (hexa-2,4-dienoic acid), biphenyl-2-ol (o-phenylphenol), zinc pyrithione, inorganic sulphites, hydrogensulphites, chlorobutanol, 4-hydroxybenzoic acid, 3-acetyl-6-methylpyran-2,4 (3h)-dione (dehydroacetic acid) and its salts, formic acid and its sodium salt, 3,3′-dibromo-4,4′-hexamethylenedioxydibenzamidine (dibromohexamidine) and its salts (including isethionate), thiomersal (inn), phenylmercuric salts (including borate), 10-undecenoic acid and salts, hexetidine, 5-bromo-5-nitro-1,3 dioxane, bronopol, 2,4-dichlorobenzyl alcohol, triclocarban, 4-chloro-m-cresol, triclosan, 4-chloro-3,5-xylenol, 3,3′-bis(1-hydroxymethyl-2,5-dioxoimidazolidin-4-yl)-1, 1′-methylenediurea, poly(1-hexamethylenebiguanide hydrochloride), 2-phenoxyethanol, hexamethylenetetramine (methenamine), methenamine 3-chloroallylochloride, 1-(4-chlorophenoxy)-1-(imidazol-1-yl) 3,3-dimethylbutan-2-one, 1,3-bis(hydroxymethyl)-5,5-dimethylimidazolidine-2,4-dione, benzyl alcohol, 1-hydroxy-4-methyl-6(2,4,4-trimethylpentyl)-2-pyridon, 6,6-dibromo-4,4-dichloro-2,2′-methylenediphenol: bromochlorophen, 4-isopropyl-m-cresol, mixture of 5-chloro-2-methyl-isothiazol-3(2h)-one and 2-methylisothiazol-3(2h)-one with magnesium chloride and magnesium nitrate, 2 benzyl-4-chlorophenol, 2-chloroacetamide, chlorhexidine (inn) and its digluconate, diacetate and dihydrochloride, 1-phenoxypropan-2-ol, alkyl (c12-c22) trimethyl ammonium, bromide and chloride, 4,4-dimethyl-1,3-oxazolidine, n-(hydroxymethyl)-n-(dihydroxymethyl-1,3-dioxo-2,5-imidazolinidyl-4)-n′-(hydroxymethyl) urea, 1,6-di(4-amidinophenoxy)-n-hexane (hexamidine) and its salts (including isethionate and p-hydroxy-benzoate, glutaraldehyde (pentane-1,5-dial), 5-ethyl-3,7-dioxa-1-azabicyclo [3.3.0]octane, 3-(p-chlorophenoxy)-propane-1,2-diol (chlorphenesin), sodium hydroxymethylamino acetate (sodium hydroxymethylglycinate), silver chloride (e.g., deposited on titanium dioxide), benzethonium chloride, benzalkonium chloride, bromide and saccharinate, benzylhemiformal, iodopropynyl butyl-carbamate, 3-iodo-2-propynylbutylcarbamate, methylisothiazolinone, and combinations thereof. Antimicrobial preservatives are provided in several embodiments. Anti-fungal and/or anti-bacterial properties may be achieved in several embodiments.

In some embodiments, a formulation described herein is provided in a kit together with other skin care or cosmetic products, along with instructions for use. Methods of making the formulations are provided herein and include, in some embodiments, adding the ingredients to water in a sanitized vessel and mixing until the ingredients are hydrated and uniform. The pH may be adjusted in one embodiment until it reaches about 5.5-7.5 (e.g., 6-7) at 25 degrees Celsius. Several embodiments use extracts, which may be obtained, for example, by the technique of extraction or by in vitro plant cell culture. Extraction includes, but is not limited to, reflux extraction, steeping, decoction, lixiviation, maceration, supercritical fluid extraction, sonication, microwave/ultrasound extraction, solid-phase micro-extraction, pressurized-liquid extraction, solid-phase extraction, and/or surfactant-mediated techniques. extraction solvents include but are not limited to: water, alcohols, and fats/oils. Examples include but are not limited to water, propylene glycol, polyethylene glycol, butylene glycol, glycerin, glycerides, diglycol ethers, diglycols, and combinations thereof. Components may be isolated and/or purified before or after extraction in some embodiments.

Where percentages are provided for agents, ingredients and compounds, they can be % m/m, % m/w, % w/w, % m/v, % v/v and variations thereof with respect to the formulation as a whole, unless otherwise indicated.

The use of agents, ingredients and compounds may be used interchangeably herein. In several embodiments, reference to the term “based” includes the recited agent, ingredient or compounds. For example, a vitamin E-based compound includes vitamin E itself or, as another example, a vitamin D-based compound includes vitamin D itself. As used herein, the terms composition and formulation can be used interchangeably. Salt forms of the acids identified herein may be used instead of or in addition to the acid.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

With respect to the use of plural and/or singular terms herein, those having skill in the art can translate from the plural to the singular and/or from the singular to the plural as is appropriate to the context and/or application.

The section headings used herein are for organizational purposes only and are not to be construed as limiting the subject matter described.

In general, terms used herein, and especially in the appended claims (including, for example, summaries of the appended claims) are generally intended, unless otherwise expressly specified, as “open” terms (for example, the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. For example, “an” agent can include one, two or several ingredients (and not necessarily a single ingredient). Disclosure of formulations and methods that “comprise” or “include” ingredients or steps also provides support for such formulations and methods to “consist of” or “consist essentially of” those ingredients or steps, when the latter phrases are used in the claims and in the context of such claims. As an example, disclosure herein of a composition that comprises or includes ingredient 1 and ingredient 2 may include additional ingredients or may consist or consist essentially of ingredient 1 and ingredient 2, if the phrases “consist of” or “consist essentially of” are recited in the claims and in the context of such claims.

In addition, even if a specific number of an introduced claim element is explicitly recited, such recitation should be interpreted to mean at least the recited number of elements (for example, the bare recitation of “two elements,” without other modifiers, means at least two elements, or two or more elements). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). Additionally, any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, the disclosure is also thereby described in terms of any individual member or subgroup of members of the Markush group.

For any and all purposes, such as in terms of providing a written description, all ranges disclosed herein also encompass any sub-ranges and combinations of sub-ranges thereof, as well as the endpoints of the range or sub-range. All language such as “up to,” “at least,” “greater than,” “less than,” and the like include the number recited and refer to ranges which can be subsequently broken down into sub-ranges as discussed above. Finally, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth. The phrases “and ranges in between” can include ranges that fall in between the numerical values listed. For example, “1, 2, 3, 10, and ranges in between” can include 1-10, 1-3, 2-10, etc.

While various aspects and embodiments have been disclosed herein, other aspects and embodiments will be apparent to those skilled in the art. The various aspects and embodiments disclosed herein are for purposes of illustration and are not intended to be limiting, with the scope and spirit being indicated by, for example, the following claims.

For the methods disclosed herein, the functions performed in the processes and methods may be implemented in differing order. The disclosure of methods or uses may also include instructing the method or use (for example, in instructions for use).

What is claimed is:

1. A formulation for stimulating a vitamin D based compound and enhancing the microbiome, the formulation comprising:
a prebiotic;
a probiotic;
a postbiotic;
a plurality of ingredients to stimulate the vitamin D based compound without containing vitamin D; and
a synthetic preservative,
wherein the formulation is provided as a topical formulation and is configured for use on a user's skin for both stimulating the vitamin D based compound and enhancing the microbiome.

2. The formulation of claim 1, wherein the formulation is configured as a spray for stimulating the vitamin D based compound in at least one of keratinocytes and fibroblasts.

3. The formulation of claim 1, wherein the formulation stimulates the vitamin D based compound in dark conditions.

4. The formulation of claim 1, wherein the formulation is configured to reduce Propionibacterium acnes.

5. The formulation of claim 1, wherein the synthetic preservative comprises phenoxyethanol.

6. The formulation of claim 1, wherein the formulation is configured to increase beneficial skin microorganisms and reduce harmful skin microorganisms.

7. The formulation of claim 1,
wherein the prebiotic comprises a Lycium barbarum fruit extract;
wherein the probiotic comprises one or more ferments comprising a Lactobacillus ferment;
wherein the postbiotic comprises a squalane;
wherein the plurality of ingredients to stimulate the vitamin D compound comprises a glutamylamidoethyl imidazole, a fructooligosaccharide, a glycoprotein, and a bioflavonoid.

8. The formulation of claim 7, wherein the Lycium barbarum fruit extract is present in an amount between 0.001-2% of the formulation.

9. The formulation of claim 7, wherein the glutamylamidoethyl imidazole present in an amount between 0.001-2% of the formulation.

10. The formulation of claim 1, further comprising a Opuntia Ficus-Indica stem extract.

11. A formulation for topical use for stimulating a vitamin D based compound and enhancing the microbiome, the formulation comprising:
a probiotic, wherein the probiotic comprises a Lactobacillus ferment;
a postbiotic, wherein the postbiotic comprises squalane;
a synthetic preservative, and
fructooligosaccharides, glycoproteins, bioflavonoids, glycerin, and a Opuntia Ficus-Indica extract;
wherein the formulation is provided as a topical formulation and is configured for use on a user's skin for both stimulating the vitamin D based compound and enhancing the microbiome.

12. The formulation of claim 11,
wherein the probiotic present in an amount between 0.001% and 3% of the formulation.

13. The formulation of claim 11, further comprising one or more of glucose, guar gum and xanthan gum.

14. The formulation of claim 11, further comprising one or more ingredients selected from the group consisting of: a hydrolyzed pea protein, glutamylamidoethyl imidazole, polyglyceryl-10 oleate, sodium hydroxide, and pentylene glycol.

15. The formulation of claim 11, wherein the formulation is configured to increase beneficial skin microorganisms and reduce harmful skin microorganisms.

16. The formulation of claim 11, wherein the formulation is configured as a spray for stimulating the vitamin D based compound in at least one of keratinocytes and fibroblasts.

17. The formulation of claim 11, wherein the formulation stimulates the vitamin D based compound in dark conditions, and wherein the formulation does not contain Vitamin D.
